(19) United States
(12) Patent Application Publication
(10) Pub. No.: US 2026/0263424 A1
(43) Pub. Date: Sep. 10, 2026

(54) Formulations of 2-Arylbenzimidazole Compounds

(71) Applicant: Tranquis Therapeutics, Inc., Palo Alto, CA (US)

(72) Inventors: Andrew Lam, San Jose, CA (US); James Coghill, Reading (GB); Yogesh Bansal, Slough (GB)

(21) Appl. No.: 18/875,703

(22) PCT Filed: Jun. 14, 2023
(86) PCT No.: PCT/US2023/025331

§ 371 Date: Dec. 16, 2024

Related U.S. Application Data
(60) Provisional application No. 63/353,256, filed on Jun. 17, 2022.

(51) Int. Cl. A61K 31/4184; A61K 9/16; A61K 47/02; A61K 47/32

(52) U.S. Cl. CPC A61K 31/4184; A61K 9/16; A61K 47/02; A61K 47/32

(57) ABSTRACT

The present disclosure provides amorphous solid dispersions comprising 2-(4-tert-butylphenyl)-1H-benzimidazole (compound 1) or a pharmaceutically acceptable salt thereof (e.g., a spray-dried dispersion (SDD) or hot-melt extrusion (HME) of compound 1). Also provided are pharmaceutical compositions and pharmaceutical dosage forms including the subject amorphous solid dispersions of compound 1. Also provided are kits including the subject pharmaceutical dosage forms and methods of delivering the subject pharmaceutical dosage forms to a subject to achieve an enhanced maximum blood plasma concentration (Cₘₐₓ) for compound 1 with respect to a control formulation of compound 1, and/or an area under the curve (AUC) for compound 1 and/or a metabolite of compound 1 (e.g., compound 2 as described herein) that is greater than that achieved with a control formulation of compound 1.

### 1. CROSS-REFERENCE TO RELATED APPLICATIONS

This application claims the benefit of and priority to U.S. Provisional Application No. 63/353,256, filed Jun. 17, 2022, which is hereby incorporated by reference in its entirety.

### 2. INTRODUCTION

The 2-arylbenzimidazole compound 2-(4-tert-butylphenyl)-1H-benzimidazole (compound 1), also known as TQS-168 or ZLN-005, is known to be an activator of Ppargc1α (PGC-1α) expression (Zhang et al., Diabetes 62:1297-1307 (2013)) and has the following structure:

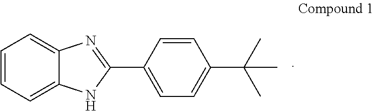 [SMILES: CC(C)(C)c1ccc(-c2nc3ccccc3[nH]2)cc1]

When administered orally at 25 mg/kg, compound 1 has been shown to suppress myeloid-mediated inflammation and reduce disease severity in murine models of neurodegenerative diseases, including Parkinson's disease, Alzheimer's disease, and amyotrophic lateral sclerosis (ALS). See U.S. Pat. No. 10,272,070

When administered orally at 25 mg/kg, compound 1 has also been shown to suppress metabolic dysfunction in microglia in older mice, inhibit inflammatory cytokine production in microglia in older mice, suppress systemic inflammation in older mice, and alleviate behavioral dysfunction in older mice. See U.S. Pat. No. 10,653,669. Compound 1 and structurally related 2-arylbenzimidazoles have also been shown to be effective in treating systemic immune activation. See WO 2021/262617.

Compound 1 is highly insoluble under aqueous conditions. In the animal model experiments reported in U.S. Pat. No. 10,272,070, compound 1 was prepared as an oral suspension and administered to experimental animals by oral gavage. Plasma and brain concentrations of the compound after administration were not reported, providing no pharmacokinetic (PK) information. In addition, although compound 1 and structurally related compounds are known to increase PGC-1α expression, the direct molecular binding partner(s) of these compounds, including whether such binding partner(s) are intracellular or extracellular, and if intracellular, whether cytoplasmic, nuclear, or mitochondrial, is unknown, presenting significant obstacles to determining PK/PD relationships.

Improved oral dosage forms including Compound 1 are of interest.

### 3. SUMMARY

The present disclosure provides amorphous solid dispersions comprising 2-(4-tert-butylphenyl)-1H-benzimidazole (compound 1) or a pharmaceutically acceptable salt thereof.

The subject amorphous solid dispersion may be prepared by spray-drying (e.g., to provide a spray-dried dispersion (SDD) formulation), or by hot-melt extrusion (e.g., to provide a hot-melt extrusion (HME) formulation). Also provided are pharmaceutical compositions and pharmaceutical dosage forms including the subject amorphous solid dispersions of compound 1 (e.g., SDD or HME formulation of compound 1). Also provided are kits including the subject pharmaceutical dosage forms and methods of delivering the subject pharmaceutical dosage forms to a subject to achieve an enhanced maximum blood plasma concentration (Cₘₐₓ) for compound 1 with respect to a control formulation of compound 1, and/or an area under the curve (AUC) for compound 1 and/or a metabolite of compound 1 (e.g., compound 2 as described herein) that is greater than that achieved with a control formulation of compound 1.

In one aspect of this disclosure, an amorphous solid dispersion (ASD) is provided comprising 2-(4-tert-butylphenyl)-1H-benzimidazole (compound 1) or a pharmaceutically acceptable salt thereof; and a pharmaceutically acceptable carrier matrix.

In a further aspect, the amorphous solid dispersion is prepared by spray-drying or hot-melt extrusion. In some specific embodiments it is prepared by spray-drying. In some specific embodiments it is prepared by hot-melt extrusion.

In some embodiments of the amorphous solid dispersion, it comprises 40% w/w or less of 2-(4-tert-butylphenyl)-1H-benzimidazole (compound 1) or a pharmaceutically acceptable salt thereof. In some particular embodiments, it comprises 30% w/w of 2-(4-tert-butylphenyl)-1H-benzimidazole (compound 1) or a pharmaceutically acceptable salt thereof. In other particular embodiments, it comprises 25% w/w of 2-(4-tert-butylphenyl)-1H-benzimidazole (compound 1) or a pharmaceutically acceptable salt thereof.

In some embodiments of the amorphous solid dispersion, the pharmaceutically acceptable carrier matrix comprises a polyvinyl caprolactam-polyvinyl acetate-polyethylene glycol graft co-polymer, a polyvinylpyrrolidone polymer, a copovidone polymer, a povidone polymer, a hydroxypropyl methyl cellulose polymer, a dimethylaminoethyl methacrylate-copolymer, a methacrylic acid-methyl methacrylate copolymer, a polyethylene glycol polymer, amorphous silicon dioxide and mixtures thereof.

In some embodiments of the amorphous solid dispersion, the pharmaceutically acceptable carrier matrix comprises a copovidone polymer, a povidone polymer, a polyvinyl caprolactam-polyvinyl acetate-polyethylene glycol graft co-polymer, amorphous silicon dioxide, or a mixture thereof.

In some embodiments of the amorphous solid dispersion, the pharmaceutically acceptable carrier matrix comprises a mixture of a polyvinyl caprolactam-polyvinyl acetate-polyethylene glycol graft co-polymer, and amorphous silicon dioxide.

In some embodiments of the amorphous solid dispersion, the pharmaceutically acceptable carrier matrix comprises a mixture of copovidone polymer, a povidone polymer, and a polyvinyl caprolactam-polyvinyl acetate-polyethylene glycol graft co-polymer.

In some embodiments of the amorphous solid dispersion, the ASD comprises 60 to 85% w/w of the pharmaceutically acceptable carrier matrix.

In some embodiments of the amorphous solid dispersion, the ASD comprises 65 to 80% w/w of the pharmaceutically acceptable carrier matrix.

In some embodiments of the amorphous solid dispersion, the ASD comprises 70% w/w of the pharmaceutically acceptable carrier matrix.

In some embodiments of the amorphous solid dispersion, the ASD comprises 75% w/w of the pharmaceutically acceptable carrier matrix.

In one aspect of this disclosure, an amorphous solid dispersion is provided comprising:
10-40% w/w of 2-(4-tert-butylphenyl)-1H-benzimidazole (compound 1) or a pharmaceutically acceptable salt thereof; and
60 to 90% w/w of a pharmaceutically acceptable carrier matrix,
   wherein the amorphous solid dispersion is prepared by spray-drying.

In a further aspect, the amorphous solid dispersion comprises:
30% w/w of 2-(4-tert-butylphenyl)-1H-benzimidazole (compound 1) or a pharmaceutically acceptable salt thereof; and
70% w/w of a pharmaceutically acceptable carrier matrix.

In some embodiments of the amorphous solid dispersion, the pharmaceutically acceptable carrier matrix comprises a polyvinyl caprolactam-polyvinyl acetate-polyethylene glycol graft co-polymer, and amorphous silicon dioxide.

In another aspect of the disclosure, the amorphous solid dispersion comprises:
10-40% w/w of 2-(4-tert-butylphenyl)-1H-benzimidazole (compound 1) or a pharmaceutically acceptable salt thereof; and
60 to 90% of a pharmaceutically acceptable carrier matrix;
   wherein the amorphous solid dispersion is prepared by hot-melt extrusion.

In a further aspect, the amorphous solid dispersion comprises:
25% w/w of 2-(4-tert-butylphenyl)-1H-benzimidazole (compound 1) or a pharmaceutically acceptable salt thereof; and
75% w/w of a pharmaceutically acceptable carrier matrix.

In some embodiments of the amorphous solid dispersion, the pharmaceutically acceptable carrier matrix comprises a mixture of:
25% w/w of a copovidone polymer;
25% w/w of a povidone polymer; and
25% w/w of a polyvinyl caprolactam-polyvinyl acetate-polyethylene glycol graft co-polymer.

In another aspect of this disclosure, a pharmaceutical composition including the subject amorphous solid dispersion of compound 1 (e.g., SDD or HME formulation of compound 1) and one or more pharmaceutically acceptable excipients is provided.

In some further embodiments of the pharmaceutical composition, it comprises 30 to 50% w/w of the amorphous solid dispersion. In some embodiments of the pharmaceutical composition, it comprises 35 to 45% w/w of the amorphous solid dispersion. In some embodiments of the pharmaceutical composition, it comprises 40% w/w of the amorphous solid dispersion.

In some embodiments of the pharmaceutical composition, the one or more pharmaceutically acceptable excipients is selected from diluents, binders, disintegrants, lubricants, glidants, surfactants, solubilizers, plasticizers, stabilizing agents, antioxidants, sweeteners, and any combination thereof.

In some embodiments of the pharmaceutical composition, the composition comprises a diluent. In some further embodiments, the pharmaceutical composition comprises 40 to 70% w/w of the diluent. In some further embodiments, the pharmaceutical composition comprises 45 to 65% w/w of the diluent. In some further embodiments, the pharmaceutical composition comprises 50 to 60% w/w of the diluent.

In some embodiments of the pharmaceutical composition, the composition comprises a disintegrant. In some further embodiments, the pharmaceutical composition comprises 10% w/w of the disintegrant. In some further embodiments, the pharmaceutical composition comprises comprising 2 to 8% w/w of the disintegrant. In some further embodiments, the pharmaceutical composition comprises 4 to 6% w/w of the disintegrant.

In some embodiments of the pharmaceutical composition, the composition comprises a lubricant. In some further embodiments, the pharmaceutical composition comprises 0.5 to 2% w/w of the lubricant. In some further embodiments, the pharmaceutical composition comprises 0.5 to 1.5% w/w of the lubricant.

In some embodiments of the pharmaceutical composition, the composition comprises a glidant. In some further embodiments, the pharmaceutical composition comprises 0.5 to 2.5% w/w of the glidant. In some embodiments the pharmaceutical composition comprises 1 to 2% w/w of the glidant.

In some embodiments of the pharmaceutical composition, the composition comprises a sweetener. In some embodiments, it comprises 1 to 5% w/w of the sweetener. In some embodiments, it comprises 1 to 2.5% w/w of the sweetener.

In some embodiments of the pharmaceutical composition, the composition comprises:
30 to 50% w/w of the amorphous solid dispersion;
40 to 70% w/w of a diluent;
1 to 10% w/w of a disintegrant;
0.5 to 2% w/w of a lubricant; and
0.5 to 2% w/w of a glidant.

In some embodiments of the pharmaceutical composition, the composition comprises:
35 to 45% w/w of the amorphous solid dispersion;
50 to 60% w/w of a diluent;
4 to 6% w/w of a disintegrant;
0.5 to 1.5% w/w of a lubricant; and
1 to 2% w/w of a glidant.

In some further embodiments of the pharmaceutical composition, the composition comprises 1 to 5% w/w of a sweetener.

In some embodiments of the pharmaceutical composition, the diluent is selected from the group consisting of microcrystalline cellulose, dicalcium phosphate, cellulose, compressible sugars, dibasic calcium phosphate dehydrate, lactose, lactose monohydrate, lactose anhydrous, mannitol, tribasic calcium phosphate, and combinations thereof.

In some embodiments of the pharmaceutical composition, the disintegrant is selected from the group consisting of croscarmellose sodium, crospovidone, modified corn starch, pregelatinized starch, sodium starch glycolate, and combinations thereof.

In some embodiments of the pharmaceutical composition, the glidant is selected from the group consisting of colloidal silicon dioxide, talc, and combinations thereof.

In some embodiments of the pharmaceutical composition, the lubricant is selected from the group consisting of sodium stearyl fumarate, calcium stearate, magnesium stearate, polyethylene glycol, stearic acid, and combinations thereof.

In some embodiments of the pharmaceutical composition, the sweetener is an artificial sweetener.

Another aspect of this disclosure includes a pharmaceutical dosage form including the subject pharmaceutical composition (e.g., as described herein). In another aspect of this disclosure, provided is a pharmaceutical dosage form comprising the amorphous solid dispersion or the pharmaceutical composition. In another aspect of this disclosure, provided is a pharmaceutical dosage form comprising a pharmaceutical composition.

In some embodiments of the pharmaceutical dosage form, 2-(4-tert-butylphenyl)-1H-benzimidazole (compound 1) is present in an amount of from 50 mg to 500 mg.

In some embodiments of the pharmaceutical dosage form, 2-(4-tert-butylphenyl)-1H-benzimidazole (compound 1) is present in an amount of from 100 mg to 450 mg.

In some embodiments of the pharmaceutical dosage form, 2-(4-tert-butylphenyl)-1H-benzimidazole (compound 1) is present in an amount of 180 mg.

In some embodiments of the pharmaceutical dosage form, 2-(4-tert-butylphenyl)-1H-benzimidazole (compound 1) is present in an amount of 270 mg.

In some embodiments of the pharmaceutical dosage form, 2-(4-tert-butylphenyl)-1H-benzimidazole (compound 1) is present in an amount of 400 mg.

In some embodiments of the pharmaceutical dosage form, 2-(4-tert-butylphenyl)-1H-benzimidazole (compound 1) is present in an amount of 450 mg.

In some embodiments of the pharmaceutical dosage form, the dosage form is granules, a powder, an oral disintegrating tablet (ODT), or an oral suspension. In some embodiments, the dosage form is a powder. In some embodiments, the dosage form is granules. In some embodiments of the granules are obtained by dry granulation.

In some embodiments of the pharmaceutical dosage form, oral administration of said pharmaceutical dosage form to a selected human subject group produces in said subject group:
an enhanced maximum blood plasma concentration (Cₘₐₓ) for compound 1 that is greater than that achieved with a control formulation of compound 1 (e.g., MC formulation of compound 1);
an area under the curve (AUC) for compound 1 that is at greater than that achieved with a control formulation of compound 1; and
an area under the curve (AUC) for a compound 1 metabolite (e.g., TQS-621) that is greater than that achieved with a control formulation of compound 1.

In another aspect of this disclosure, provided is a kit including the subject pharmaceutical dosage form and instructions for oral administration of the dosage form, wherein the instructions indicate that the composition can be reconstituted in a food or beverage

Another aspect of this disclosure includes a method of delivering a therapeutically effective amount of compound 1 to a subject in need thereof. In a further aspect of this disclosure, the method of delivery is oral administration to a subject in need thereof. In a further aspect of this disclosure, the therapeutically effective amount of compound 1 is in a pharmaceutical dosage form.

In some embodiments, oral administration to a subject in need thereof of the pharmaceutical dosage form achieves:
an enhanced maximum blood plasma concentration (Cₘₐₓ) for compound 1 that is greater than that achieved with a control formulation of compound 1 (e.g., MC formulation of compound 1);
an area under the curve (AUC) for compound 1 that is at greater than that achieved with a control formulation of compound 1; and
an area under the curve (AUC) for a compound 1 metabolite (e.g., TQS-621) that is greater than that achieved with a control formulation of compound 1.

### 4. BRIEF DESCRIPTION OF THE DRAWINGS

These and other features, aspects, and advantages of the present disclosure will become better understood with regard to the following description, and accompanying drawings where:

FIG. 1 shows the plasma concentrations of compound 1 (referred to in figure as “TQS-168”) and metabolite compound 2 (referred to in figure as “TQS-621”) over time after a single oral dose of 180 mg SDD formulation of compound 1 (e.g., prepared according to Examples 1 and 2); a HME formulation of compound 1 (e.g., prepared according to Example 3 and 4); and a control formulation (compound 1 as a MC formulation, prepared according to Example 6) in humans.

FIGS. 2A-2B illustrates plasma concentration of compound 1 (TQS-168) over time after a single oral dose of 60 mg, 180 mg or 540 mg compound 1 MC formulation (e.g., as described in Example 6, referred to in figure as “TQS-168 powder for oral suspension”) in humans. FIG. 2A is a linear plot. FIG. 2B is a logarithmic plot.

FIGS. 3A-3B illustrates plasma concentration of metabolite compound 2 (TQS-621) over time after a single oral dose of 60 mg, 180 mg or 540 mg compound 1 MC formulation (e.g., as described in Example 6, referred to in figure as “TQS-168 powder for oral suspension”) in humans. FIG. 3A is a linear plot. FIG. 3B is a logarithmic plot.

FIGS. 4A-4B plot plasma concentration of compound 1 (TQS-168) and metabolite compound 2 (TQS-621) over time after a single oral dose of 60 mg compound 1 MC formulation (e.g., as described in Example 6) in humans. FIG. 4A is a linear plot. FIG. 4B is a logarithmic plot.

FIGS. 5A-5B plot plasma concentration of compound 1 (TQS-168) and metabolite compound 2 (TQS-621) over time after a single oral dose of 180 mg compound 1 MC formulation (e.g., as described in Example 6) in humans. FIG. 5A is a linear plot. FIG. 5B is a logarithmic plot.

FIGS. 6A-6B plot plasma concentration of compound 1 (TQS-168) and metabolite compound 2 (TQS-621) over time after a single oral dose of 540 mg compound 1 MC formulation (e.g., as described in Example 6) in humans. FIG. 6A is a linear plot. FIG. 6B is a logarithmic plot.

FIGS. 7A-7B plot plasma concentration of compound 1 (TQS-168) over time after a single dose of 60 mg, 180 mg, or 540 mg compound 1 MC formulation (e.g., as described in Example 6, referred to in figure as “TQS-168 powder for oral suspension”) in the fasted state, 90 mg compound 1 SDD formulation (e.g., as described in Examples 1-2, referred to in the figures as “spray dried dispersion (SDD) powder for oral suspension”) in the fed state, 180 mg compound 1 SDD formulation in the fasted state, or 180 mg of HME formulation (e.g., as described in Examples 3-4, referred to in the figures as “hot melt extrusion (HME) powder for oral suspension”) in the fasted state. FIG. 7A is a linear plot. FIG. 7B is a logarithmic plot.

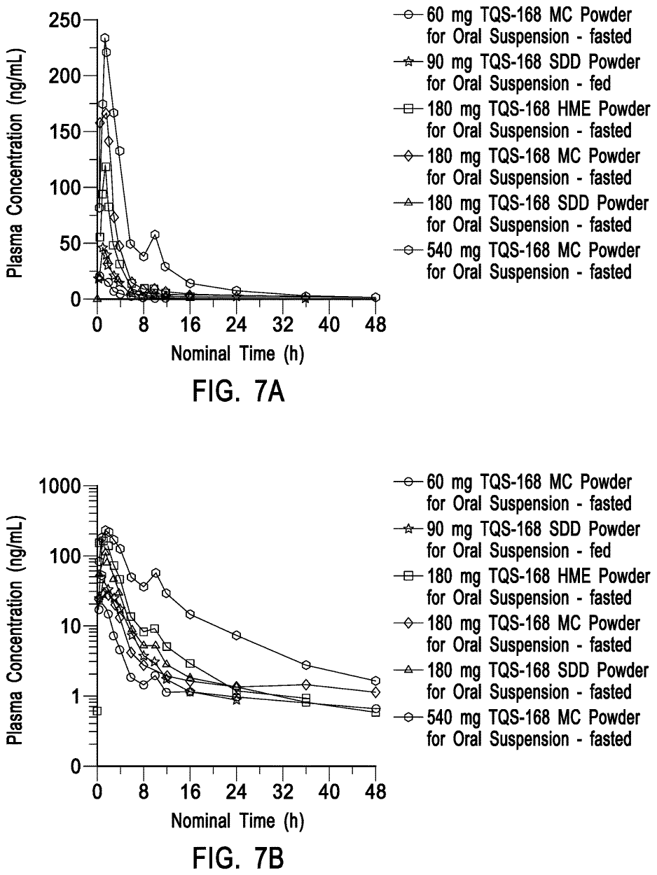

FIGS. 8A-8B plot plasma concentration of metabolite compound 2 (TQS-621) over time after a single dose of 60 mg, 180 mg, or 540 mg compound 1 MC formulation (e.g., as described in Example 6) in the fasted state, 90 mg spray compound 1 SDD formulation (e.g., as described in Examples 1-2) in the fed state, 180 mg SDD formulation in the fasted state, or 180 mg of HME formulation (e.g., as described in Examples 3-4) in the fasted state. FIG. 8A is a linear plot. FIG. 8B is a logarithmic plot.

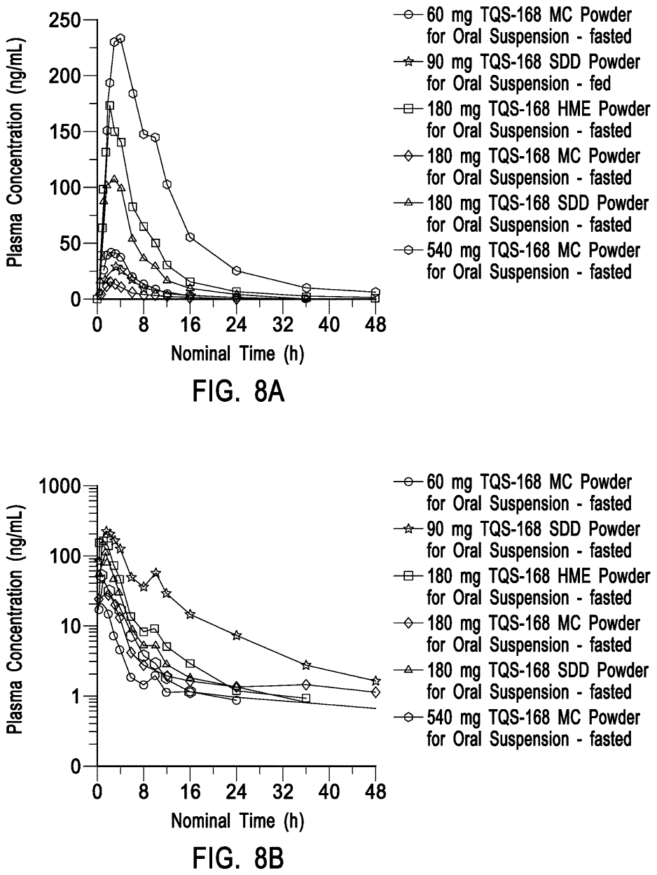

FIGS. 9A-9B plot plasma concentration of compound 1 (TQS-168) and metabolite compound 2 (TQS-621) over time after a single dose of 90 mg compound 1 SDD formulation (e.g., as described in Examples 1-2) in fed state humans. FIG. 9A is a linear plot. FIG. 9B is a logarithmic plot.

FIGS. 10A-10B plot plasma concentration of compound 1 (TQS-168) over time after a single dose of 90 mg compound 1 SDD formulation (e.g., as described in Examples 1-2) in fed and fasted state humans. FIG. 10A is a linear plot. FIG. 10B is a logarithmic plot.

FIGS. 11A-11B plot plasma concentration of metabolite compound 2 (TQS-621) over time after a single dose of 90 mg compound 1 SDD formulation (e.g., as described in Examples 1-2) in fed and fasted state humans. FIG. 11A is a linear plot. FIG. 11B is a logarithmic plot.

FIGS. 12A-12B plot plasma concentration of compound 1 (TQS-168) and metabolite compound 2 (TQS-621) over time after a single oral dose of 90 mg compound 1 SDD formulation (e.g., as described in Examples 1-2) in fasted state humans. FIG. 12A is a linear plot. FIG. 12B is a logarithmic plot.

FIGS. 13A-13B plot plasma concentration of compound 1 (TQS-168) over time after a single dose of 90 mg, 180 mg or 270 mg compound 1 SDD formulation (e.g., as described in Examples 1-2) in fasted state humans. FIG. 13A is a linear plot. FIG. 13B is a logarithmic plot.

FIGS. 14A-14B plot plasma concentration of metabolite compound 2 (TQS-621) over time after a single dose of 90 mg, 180 mg or 270 mg compound 1 SDD formulation (e.g., as described in Examples 1-2) in fasted state humans. FIG. 14A illustrates a linear plot. FIG. 14B illustrates a logarithmic plot.

FIGS. 15A-15B plot plasma concentration of compound 1 (TQS-168) over time after a single dose of 90 mg or 120 mg compound 1 SDD formulation (e.g., as described in Examples 1-2) in fed and fasted state humans respectively (Day 1). FIG. 15A is a linear plot. FIG. 15B is a logarithmic plot.

FIGS. 16A-16B plot plasma concentration of compound 1 (TQS-168) over time on Day 7 after seven consecutive days of single dose 90 mg or 120 mg compound 1 SDD formulation (e.g., as described in Examples 1-2) in fed and fasted state humans respectively. FIG. 16A is a linear plot. FIG. 16B is a logarithmic plot.

FIGS. 17A-17B plot plasma concentration of metabolite compound 2 (TQS-621) over time after a single dose of 90 mg or 120 mg TQS-168 compound 1 SDD formulation (e.g., as described in Examples 1-2) in fed and fasted state humans respectively. FIG. 17A is a linear plot. FIG. 17B is a logarithmic plot.

FIGS. 18A-18B plot plasma concentration of metabolite compound 2 (TQS-621) over time on Day 7 after seven consecutive days of single dose 90 mg or 120 mg compound 1 SDD formulation (e.g., as described in Examples 1-2) in fed and fasted state humans respectively. FIG. 18A is a linear plot. FIG. 18B is a logarithmic plot.

FIGS. 19A-19B plot plasma concentration of compound 1 (TQS-168) over time after a single dose of compound 1 MC formulation (control formulation, e.g., as described in Example 6), SDD formulation (e.g., as described in Examples 1-2), or HME formulation (e.g., as described in Examples 3-4) in varying doses in humans. FIG. 19A is a linear plot. FIG. 19B is a logarithmic plot.

FIGS. 20A-20B plot plasma concentration of compound 2 (TQS-621) over time after a single dose of compound 1 MC formulation (control formulation, e.g., as described in Example 6), SDD formulation (e.g., as described in Examples 1-2), or HME formulation (e.g., as described in Examples 3-4) in varying doses in humans. FIG. 20A is a linear plot. FIG. 20B is a logarithmic plot.

FIGS. 21A-21B plot plasma concentration of compound 1 (TQS-168) and metabolite compound 2 (TQS-621) over time after a single dose of 180 mg compound 1 SDD formulation (e.g., as described in Examples 1-2) in fasted state humans. FIG. 21A is a linear plot. FIG. 21B is a logarithmic plot.

FIGS. 22A-22B plot plasma concentration of compound 1 (TQS-168) and metabolite compound 2 (TQS-621) over time after a single dose of 180 mg compound 1 HME fonnulation (e.g., as described in Examples 3-4) in fasted state humans. FIG. 22A is a linear plot. FIG. 22B is a logarithmic plot.

FIGS. 23A-23B plot plasma concentration of compound 2 (TQS-621) over time after a single dose of compound 1 MC formulation (control formulation, e.g., as described in Example 6), SDD formulation (e.g., as described in Examples 1-2), or HME formulation (e.g., as described in Examples 3-4) in varying doses in fasted state humans. FIG. 23A is a linear plot. FIG. 23B is a logarithmic plot.

FIGS. 24A-24B plot plasma concentration of compound 1 (TQS-168) over time following consecutive single daily doses 120 mg compound 1 SDD formulation (e.g., as described in Examples 1-2) in fasted state humans. FIG. 24A is a linear plot. FIG. 24B is a logarithmic plot.

FIGS. 25A-25B plot plasma concentration of metabolite compound 2 (TQS-621) over time following consecutive single daily dose of 120 mg compound 1 SDD formulation (e.g., as described in Examples 1-2) in fasted state humans. FIG. 25A is a linear plot. FIG. 25B is a logarithmic plot.

FIGS. 26A-26B plot plasma concentration of compound 1 (TQS-168) over time after a single dose of 90 mg, 120 mg or 300 mg compound 1 SDD formulation (e.g., as described in Examples 1-2) in fed/fasted state humans (Day 1). FIG. 26A is a linear plot. FIG. 26B is a logarithmic plot.

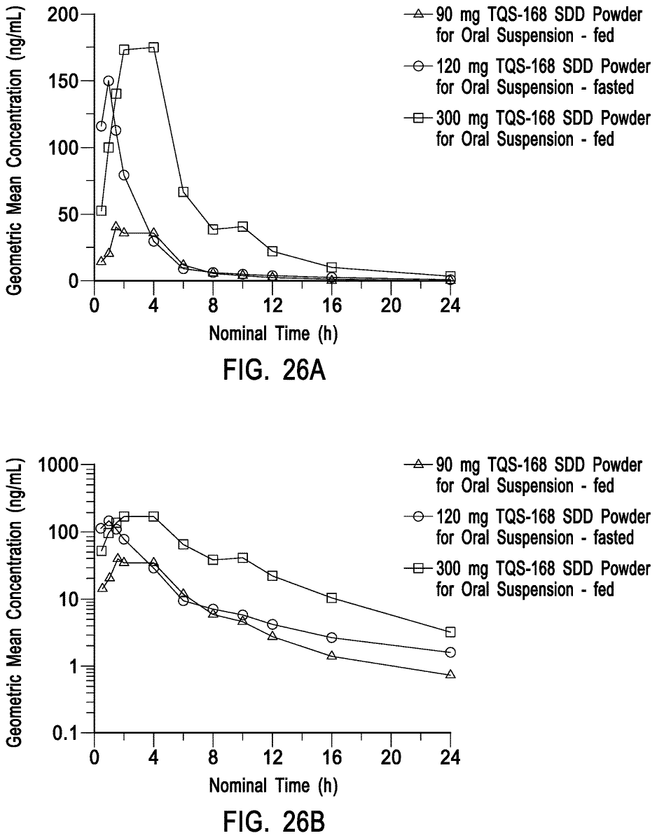

FIGS. 27A-27B plot plasma concentration of compound 1 (TQS-168) over time on Day 7 after seven consecutive days of single dose 90 mg, 120 mg, or 300 mg compound 1 SDD formulation (e.g., as described in Examples 1-2) in fed/fasted state humans respectively. FIG. 27A is a linear plot. FIG. 27B is a logarithmic plot.

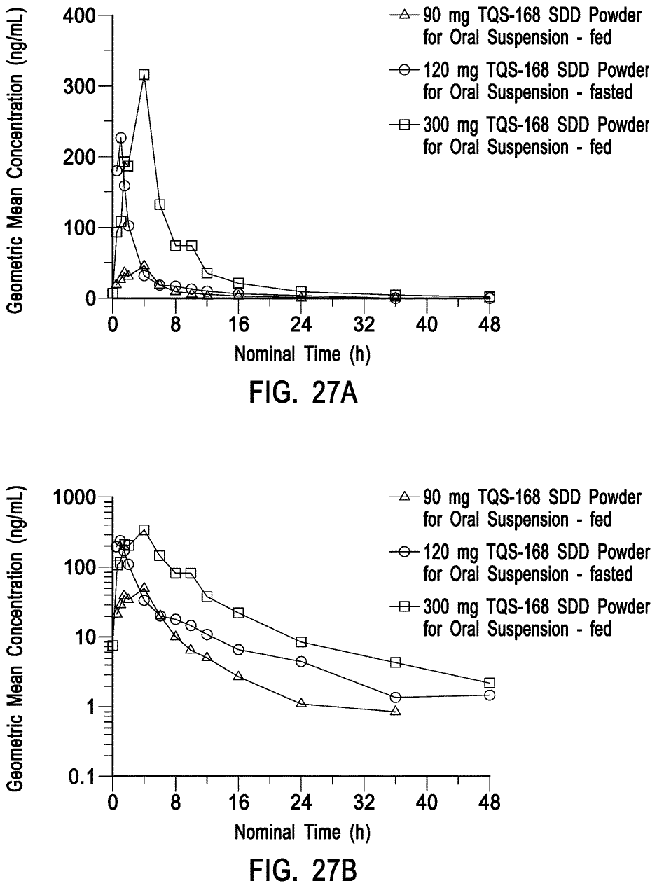

FIGS. 28A-28B plot plasma concentration of metabolite compound 2 (TQS-621) over time after a single dose of 90 mg, 120 mg or 300 mg compound 1 SDD formulation (e.g., as described in Examples 1-2) in fed/fasted state humans (Day 1). FIG. 28A is a linear plot. FIG. 28B is a logarithmic plot.

FIGS. 29A-29B plot plasma concentration of metabolite compound 2 (TQS-621) over time after seven consecutive days of single dose 90 mg, 120 mg or 300 mg compound 1 SDD formulation (e.g., as described in Examples 1-2) in fed/fasted state humans (Day 1). FIG. 29A is a linear plot. FIG. 29B is a logarithmic plot.

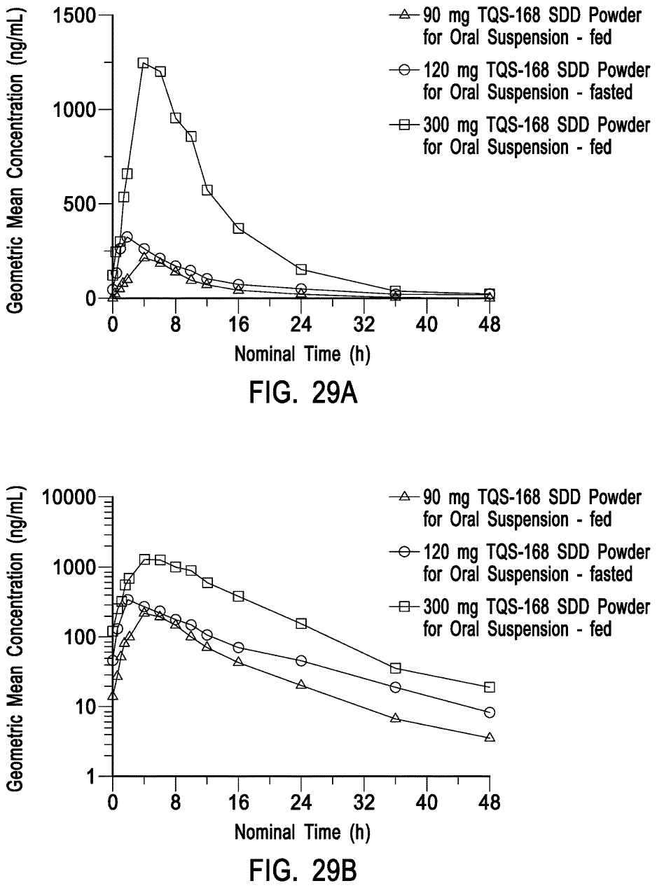

FIGS. 30A-30B plot plasma concentration of compound 1 (TQS-168) over time after a single dose of compound 1 MC formulation (control formulation, e.g., as described in Example 6), SDD formulation (e.g., as described in Examples 1-2), or HME formulation (e.g., as described in Examples 3-4) in varying doses. FIG. 30A is a linear plot. FIG. 30B is a logarithmic plot.

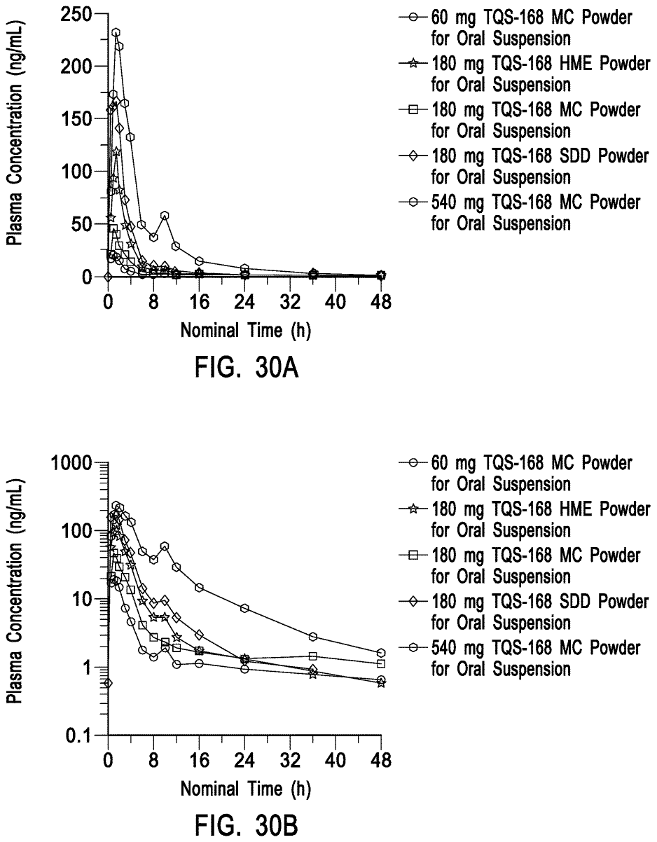

### 5. DETAILED DESCRIPTION

As summarized above, the present disclosure provides amorphous solid dispersions comprising 2-(4-tert-butylphenyl)-1H-benzimidazole (compound 1) or a pharmaceutically acceptable salt thereof. The subject amorphous solid dispersion may be prepared by spray-drying (e.g., to provide a spray-dried dispersion (SDD) formulation), or by hot-melt extrusion (e.g., to provide a hot-melt extrusion (HME) formulation). Also provided are pharmaceutical compositions and pharmaceutical dosage forms including the subject amorphous solid dispersions of compound 1 (e.g., SDD or HME formulation of compound 1). Also provided are kits including the subject pharmaceutical dosage forms and methods of delivering the subject pharmaceutical dosage forms to a subject to achieve an enhanced maximum blood plasma concentration (Cₘₐₓ) for compound 1 with respect to a control formulation of compound 1, and an area under the curve (AUC) for both compound 1 and a metabolite of compound 1 (e.g., compound 2 as described herein) that is greater than that achieved with a control formulation of compound 1.

The present disclosure further provides pharmaceutical compositions and dosage forms of an amorphous form of compound 1 having enhanced bioavailability in comparison to a control formulation of compound 1 (e.g., as described herein).

The amorphous solid dispersions of this disclosure, and pharmaceutical compositions comprising the amorphous solid dispersions are described in greater detail below. Also described are pharmaceutical dosage forms comprising the subject pharmaceutical compositions, kits including the subject pharmaceutical compositions, and methods for delivering the same to a subject.

### 5.1. Amorphous Solid Dispersions

As summarized above, this disclosure provides amorphous solid dispersions comprising 2-(4-tert-butylphenyl)-1H-benzimidazole (compound 1) or a pharmaceutically acceptable salt thereof.

The amorphous solid dispersion may be prepared by spray-drying (e.g., to provide a spray-dried dispersion (SDD) formulation) or by hot-melt extrusion (e.g., to provide a hot-melt extrusion (HME) formulation).

### 5.1.1. Compound 1 (2-(4-tert-butylphenyl)-1H-benzimidazole)

The subject amorphous solid dispersions include 2-(4-tert-butylphenyl)-1H-benzimidazole (compound 1), also known as TQS-168 or ZLN-005, is known to be an activator of Ppargc1α (PGC-1α) expression (Zhang et al., Diabetes 62:1297-1307 (2013)) and has the following structure:

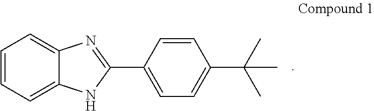 [SMILES: CC(C)(C)c1ccc(-c2nc3ccccc3[nH]2)cc1]

In some embodiments, compound 1 is in the form of a pharmaceutically acceptable salt. The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic acids. “Pharmaceutically acceptable salt” refers to pharmaceutically acceptable salts of a compound, which salts are derived from a variety of organic and inorganic counter ions well known in the art and include, salts of organic or inorganic acids, such as hydrochloride, hydrobromide, formate, tartrate, besylate, mesylate, acetate, maleate, oxalate, and the like.

### 5.1.2. Amorphous Solid Dispersions

A solid dispersion refers to a system in a solid-state comprising at least two components, wherein one component (e.g., a drug substance) is dispersed throughout the other component or components. In an embodiment, solid dispersions of compound 1 of the present application comprises compound 1 or a pharmaceutically acceptable salt, and a pharmaceutically acceptable carrier matrix.

The solid dispersions of compound 1 may be formed by any conventional technique, e.g., spray drying, co-grinding, hot melt extrusion, freeze drying, rotary evaporation, solvent evaporation, co-precipitation, lyophilization, or any suitable solvent removal process. In some embodiments, the amorphous solid dispersion is prepared by spray drying (e.g., to obtain a spray-dried dispersion (SDD) formulation of compound 1). In some embodiments, the amorphous solid dispersion is prepared by hot-melt extrusion (e.g., to obtain a hot-melt extrusion (HME) formulation of compound 1).

The compound 1 starting material used in the process for preparation of the solid dispersion may be crystalline or amorphous form. Alternatively, it may be obtained in situ from a previous processing step.

In some embodiments, compound 1 in the solid dispersion obtained is present in an amorphous form.

A solid that is in the “amorphous” solid state form means that it is in a non-crystalline state. Amorphous solids generally possess crystal-like short-range molecular arrangement, but no long-range order of molecular packing as are found in crystalline solids. The solid-state form of a solid, such as the drug substance in the amorphous dispersion, may be determined by Polarized Light Microscopy, X-Ray Powder Diffraction (XPRD), Differential Scanning calorimetry (DSC), or other standard techniques known to those of skill in the art. In some embodiments, the amorphous solid contains compound 1 in a substantially amorphous solid-state form, e.g., at least about 80% of compound 1 in the dispersion is in an amorphous form, such as at least about 90% of compound 1 in the dispersion is in an amorphous form, or at least about 95% of compound 1 in the dispersion is in amorphous form.

In some embodiments, at least about 90% (e.g., at least 95%, 96%, 97%, 98%, 99%, 99.5%, or even 99.9%, such as from 90% to 99.9%, from 90% to 99.5%, from 90% to 99%, from 90% to 98%, from 90% to 97%, from 90% to 96%, from 90% to 95%, from 95% to 99.9%, from 95% to 99.5%, from 95% to 99%, from 95% to 98%, from 95% to 97%, and from 95% to 96%) of compound 1 is in amorphous form.

The solid dispersion can be in a single phase such as substitutional or interstitial amorphous solutions; or it can be a two-phase system such as eutectics, amorphous drug and amorphous carrier dispersions. Solid solutions are a resultant single phase upon dispersion of two compounds in each other, at their molecular level.

The inventors of the present application have found that a composition comprising amorphous solid dispersion of compound 1 comprising at least one pharmaceutically acceptable carrier, can increase the maximum blood plasma concentration (Cₘₐₓ) and area under the curve (AUC) of compound 1 as compared to a control formulation of compound 1 (e.g., as described herein).

In some embodiments, the amorphous solid dispersion of compound 1 includes at least one pharmaceutically acceptable carrier in the carrier matrix.

According to some embodiments of the disclosure, the carrier matrix comprises a polyvinyl caprolactam-polyvinyl acetate-polyethylene glycol graft co-polymer (e.g., soluplus), a polyvinylpyrrolidone polymer (e.g., PVP K30), a copovidone polymer (e.g., PVP VA64 or Kollidon VA64), a povidone polymer (e.g., Kollidon 17PF), a hydroxypropyl methyl cellulose polymer (e.g., HPMC AS), a dimethylaminoethyl methacrylate-copolymer (e.g., Eudragit EPO), a methacrylic acid-methyl methacrylate copolymer (e.g., Eudragit L100), a polyethylene glycol polymer (e.g., PEG 8000), amorphous silicon dioxide (e.g., Syloid® 244 FP) and mixtures thereof.

According to the embodiments of the invention, the hydroxypropyl methylcellulose acetate succinate (HPMC-AS) comprises various types, such as LF, LG, MF, MG, HF and HG, etc., the first letters L, M and H of the type's names mean the pH level at the beginning of dissolution of HPMC-AS. For example, L refers to low level (e.g., HPMC-AS begins to be dissolved when the pH value is more than 5.5), M refers to middle level (e.g., HPMC-AS begins to be dissolved when the pH value is more than 6.0), H refers to high level (e.g., HPMC-AS begins to be dissolved when the pH value is more than 6.5). The second letters F and G refer to the particle size of HPMC-AS, where F refers to fine powder, and G refers to granular. In some embodiments, the type of HPMC-AS is LF; in some embodiments, the type of HPMC-AS is MF; in some embodiments, the type of HPMC-AS is HG.

More generally, any convenient carrier polymer can find use in subject amorphous solid dispersion formulations. In some embodiments, the carrier polymers include but are not limited to, cellulose acetate phthalate, cellulose acetate trimellitate, cellulose acetate succinate, methyl cellulose phthalate, ethylhydroxymethylcellulose phthalate, hydroxypropylmethylcellulose phthalate (HPMCP), hydroxypropylmethyl cellulose acetate succinate (HPMC-AS), hydroxypropylmethyl cellulose acetate maleate, hydroxypropylmethylcellulose trimellitate, carboxymethylethyl cellulose, polyvinyl butyrate phthalate, polyvinyl acetate phthalate, a methacrylic acid/ethyl acrylate copolymer and a methacrylic acid/methyl methacrylate copolymer. In some embodiments, the polymer is selected from HPMCP, HPMC-AS, hydroxypropylmethyl cellulose acetate maleate and hydroxypropylmethylcellulose trimellitate.

In some embodiments, the carrier polymer is selected from the group consisting of hydroxypropyl methylcellulose, hydroxypropyl cellulose, hydroxyethyl cellulose, polyvinylpyrrolidone (povidone), poly(vinylpyrrolidone/vinylacetate) (copovidone), polyvinylcaprolactam/polyvinylacetate/polyethylene glycol graft copolymer, polyethylene glycol/polyvinyl alcohol graft copolymer, polyethylene oxide, polypropylene oxide, copolymers of ethylene oxide and propylene oxide, polyvinyl alcohol, partially saponified polyvinylalcohol, macrogolglycerol hydroxystearate, polyethylene glycol, and maltodextrins. In some embodiments, the carrier polymer is a copovidone polymer.

In some embodiments, the carrier matrix comprises a copovidone polymer, a povidone polymer, a polyvinyl caprolactam-polyvinyl acetate-polyethylene glycol graft co-polymer, amorphous silicon dioxide (e.g., Syloid® 244 FP), or a mixture thereof.

In some embodiments, carrier matrix comprises a mixture of a polyvinyl caprolactam-polyvinyl acetate-polyethylene glycol graft co-polymer (e.g., soluplus), and amorphous silicon dioxide (e.g., Syloid® 244 FP).

In some embodiments, carrier matrix comprises a mixture of copovidone polymer, a povidone polymer, and a polyvinyl caprolactam-polyvinyl acetate-polyethylene glycol graft co-polymer.

In some embodiments, the amorphous solid dispersion of the compound 1, and pharmaceutically acceptable carrier matrix have a weight ratio of the compound 1 to the carrier matrix of from 1:6 to 1:1 (e.g., from 1:6 to 1:2, from 1:6 to 1:2.5, from 1:6 to 1:3, from 1:6 to 1:3.5, from 1:6 to 1:4, from 1:6 to 1:4.5, from 1:6 to 1:5, from 1:5 to 1:2, from 1:5 to 1:2.5, from 1:5 to 1:3, from 1:5 to 1:3.5, from 1:5 to 1:4, from 1:5 to 1:4.5, from 1:5 to 1:1.5, from 1:4 to 1:1.5, from 1:4 to 1:2, from 1:4 to 1:2.5, from 1:4 to 1:3, from 1:4 to 1:3.5, from 1:3 to 1:1.5, from 1:3 to 1:2, from 1:3 to 1:2.5, and from 1:2 to 1:1.5).

In certain embodiments, compound 1 and carrier matrix are present in a ratio of from 1:1 to 1:6 (w/w), 1:1 to 1:4 (w/w), such as in the ratio of 1:2 to 1.3 (w/w).

Solid dispersions of the present invention optionally may include one or more solubilizers, i.e., additives which increase solubility of the pharmaceutical active ingredient in the solid dispersion or additives which act as pore-forming agents in the solid dispersion. Suitable solubilizers for use in compositions of the present disclosure include mannitol, transcutol, polyvinylalcohol, hydroxypropyl methylcellulose, hydroxypropyl cellulose, methyl cellulose, polyvinylpyrrolidone, glycofurol and transcutol. The concentration of solubilizer ranges from about 0.5% to about 30% w/w of carrier concentration.

The amorphous solid dispersions of the present disclosure optionally may include one or more surfactants. Surfactants are compounds which are capable of improving the wetting of the pharmaceutical active ingredient and/or enhancing the dissolution. The surfactants can be selected from hydrophilic surfactants or lipophilic surfactants or mixtures thereof. The surfactants can be anionic, nonionic, cationic, and zwitterionic surfactants. Surfactants according to the present disclosure include, but not limited to, polyoxyethylene alkylaryl ethers such as polyoxyethylene lauryl ether, polyoxyethylene cetyl ether, polyoxyethylene stearyl ether; polyethylene glycol fatty acid esters such as PEG monolaurate, PEG dilaurate, PEG distearate, PEG dioleate; polyoxyethylene sorbitan fatty acid ester such as polysorbate 40, polysorbate 60, polysorbate 80; sorbitan fatty acid mono esters such as sorbitan monolaurate, sorbitan monooleate, sorbitan sesquioleate, sorbitan trioleate, sodium lauryl sulfate, sodium dioctyl sulfosuccinate (DOSS), lecithin, stearylic alcohol, cetostearylic alcohol, cholesterol, polyoxyethylene ricin oil, polyoxyethylene fatty acid glycerides, cremophor RH 40, and the like or combinations thereof. The concentration of surfactant ranges from about 0.1% to about 10% w/w of carrier concentration.

In some embodiments herein, the percentage loading of compound 1 in the solid dispersion is from 1% to 40% (w/w) (e.g., from 1% to 35%, from 10% to 35%, from 10% to 30%, from 20% to 30%, from 21% to 30%, from 22% to 30%, from 23% to 30%, from 24% to 30%, from 25% to 30%, from 26% to 30%, from 27% to 30%, from 28% to 30%). In some embodiments, the percentage loading of compound 1 is from 15% to 35% (w/w) (e.g., from 15% to 34%, from 15% to 33%, from 15% to 32%, from 15% to 31%, from 15% to 30%, from 20% to 30%, from 20% to 25%, from 25% to 30%).

In some embodiments, the percentage loading of compound 1 in the solid dispersion is from 20-30% w/w. In some embodiments, the percentage loading of compound 1 in the solid dispersion is from 25-30% w/w, such as 25%, 26%, 27%, 28%, 29%, or 30% w/w compound 1. In some embodiments, the percentage loading of compound 1 in the solid dispersion is 25% w/w. In some embodiments, the percentage loading of compound 1 in the solid dispersion is 30% w/w.

In some embodiments herein, the percentage loading of compound 1 in the solid dispersion is from 1% to 40% (w/w) (e.g., from 1% to 35%, from 10% to 35%, from 10% to 30%, from 20% to 30%, from 21% to 30%, from 22% to 30%, from 23% to 30%, from 24% to 30%, from 25% to 30%, from 26% to 30%, from 27% to 30%, from 28% to 30%, from 30% to 40%, from 35% to 40%). In some embodiments, the percentage loading of compound 1 is from 15% to 35% (w/w) (e.g., from 15% to 34%, from 15% to 33%, from 15% to 32%, from 15% to 31%, from 15% to 30%, from 20% to 30%, from 20% to 25%, from 25% to 30%).

In some embodiments, the percentage of the pharmaceutically acceptable carrier matrix in the solid dispersion is from 1% to 90% (w/w) (e.g., from 1% to 19%, from 10% to 19%, from 10% to 20%, from 10% to 30%, from 10% to 40%, from 10% to 50%, from 10% to 60%, from 10% to 70%, from 10% to 80%, from 10% to 90%, from 20% to 30%, from 20% to 40%, from 20% to 50%, from 20% to 60%, from 20% to 70%, from 20% to 80%, from 20% to 90%, from 21% to 30%, from 21% to 34%, from 21% to 40%, from 21% to 50%, from 21% to 60%, from 21% to 70%, from 21% to 80%, from 21% to 90%, from 30% to 40%, from 30% to 50%, from 30% to 60%, from 30% to 70%, from 30% to 80%, from 30% to 90%, from 36% to 40%, from 36% to 49%, from 36% to 60%, from 36% to 70%, from 36% to 80%, from 36% to 90%, from 40% to 50%, from 40% to 60%, from 40% to 70%, from 40% to 80%, from 40% to 90%, from 50% to 60%, from 50% to 70%, from 50% to 80%, from 50% to 90%, 51% to 60%, from 51% to 70%, from 51% to 80%, from 51% to 90%, from 60% to 70%, from 60% to 80%, from 60% to 90%, from 70% to 80%, and from 70% to 90%). In some embodiments, the percentage of the pharmaceutically acceptable carrier matrix in the solid dispersion is from 60% to 85% (w/w) (e.g., from 60% to 80%, from 60% to 75%, from 60% to 70%, from 65% to 85%, from 65% to 80%, from 65% to 80%, from 65% to 77%, from 65% to 76%, from 65% to 75%, from 66% to 75%, from 66% to 75%, from 67% to 75%, from 68% to 75%, and from 70% to 75%).

In some embodiments, the percentage of the pharmaceutically acceptable carrier matrix in the solid dispersion is from 60% to 85% (w/w) (e.g., from 65% to 80%, from 65% to 75%, and from 70 to 75). In some embodiments, the percentage of the pharmaceutically acceptable carrier matrix in the solid dispersion is from 65% to 80% (w/w). In some embodiments, the percentage of the pharmaceutically acceptable carrier matrix in the solid dispersion is 70% (w/w). In some embodiments, the percentage of the pharmaceutically acceptable carrier matrix in the solid dispersion is 75% (w/w).

### 5.1.3. Spray-Dried Dispersion (SDD) Formulations

In some embodiments, amorphous solid dispersions of compound 1 are obtained by a spray drying process. Spray dried dispersions are obtained by dissolving drug and the carrier polymer in an organic solvent and then spray-drying the mixture to obtain a spray-dried dispersion (SDD). The formulation and process conditions are chosen so that the solvent quickly evaporates from the droplets, allowing insufficient time for phase separation or crystallization. Accordingly, in some embodiments, there is provided an amorphous solid dispersion comprising compound 1 and a pharmaceutically acceptable carrier matrix, wherein the amorphous solid dispersion is prepared by spray-drying (e.g., to obtain a spray-dried dispersion (SDD) formulation). In an embodiment there is provided a spray-dried dispersion comprising 10-30% w/w of compound 1 and 70-90% of a pharmaceutically acceptable carrier matrix, wherein the amorphous solid dispersion is prepared by spray-drying (e.g., to obtain a spray-dried dispersion (SDD) formulation).

The term “spray dried dispersion” (SDD) is a single phase amorphous molecular dispersion of a drug in a polymer matrix. It is a solid solution prepared by dissolving the drug and a polymer in a solvent (e.g., methanol, 2-propanol or the like) and spray drying the solution. The solvent rapidly evaporates from droplets which rapidly solidifies the polymer and drug mixture trapping the drug in amorphous form as an amorphous molecular dispersion.

Accordingly, in an embodiment there is provided a spray-dried dispersion (SDD) formulation comprising compound 1 and a pharmaceutically acceptable carrier matrix. In an embodiment there is provided a spray-dried dispersion comprising 10-30% w/w of compound 1 and 70-90% of a pharmaceutically acceptable carrier matrix.

In some embodiments, the percentage loading of compound 1 in the spray-dried dispersion (SDD) formulation is from 15-40% w/w. In some embodiments, the percentage loading of compound 1 in the spray-dried dispersion (SDD) formulation is from 20-40% w/w, such as 20%, 21%, 22%, 23%, 24%, 25%, 26%, 27%, 28%, 29%, 30%, 31%, 32%, 33%, 34%, 35%, 36%, 37%, 38%, 39%, or 40% w/w compound 1. In some embodiments, the percentage loading of compound 1 in the spray-dried dispersion (SDD) formulation is 30% w/w.

In some embodiments, the percentage of the pharmaceutically acceptable carrier matrix in the spray-dried dispersion (SDD) formulation is from 60% to 85% (w/w) (e.g., from 70% to 80%, and from 70% to 75%). In some embodiments, the percentage of the pharmaceutically acceptable carrier matrix in the spray-dried dispersion (SDD) formulation is from 70% to 75% (w/w), such as 70%, 71%, 72%, 73%, 74%, or 75% (w/w). In some embodiments, the percentage of the pharmaceutically acceptable carrier matrix in the solid dispersion is 70% (w/w).

In some embodiments of the spray-dried dispersion (SDD) formulation, the pharmaceutically acceptable carrier matrix comprises a polyvinyl caprolactam-polyvinyl acetate-polyethylene glycol graft co-polymer (e.g., soluplus), a polyvinylpyrrolidone polymer (e.g., PVP K30), a copovidone polymer (e.g., PVP VA64 or Kollidon VA64), a povidone polymer (e.g., Kollidon 17PF), a hydroxypropyl methyl cellulose polymer (e.g., HPMC AS), a dimethylaminoethyl methacrylate-copolymer (e.g., Eudragit EPO), a methacrylic acid-methyl methacrylate copolymer (e.g., Eudragit L100), a polyethylene glycol polymer (e.g., PEG 8000), amorphous silicon dioxide (e.g., Syloid® 244 FP) and mixtures thereof.

In some embodiments of the spray-dried dispersion (SDD) formulation, the pharmaceutically acceptable carrier matrix comprises a polyvinyl caprolactam-polyvinyl acetate-polyethylene glycol graft co-polymer (e.g., soluplus), and amorphous silicon dioxide (e.g., Syloid® 244 FP).

In some embodiments of the spray-dried dispersion (SDD) formulation, the ratio of amorphous silicon dioxide (e.g., Syloid® 244 FP) to polyvinyl caprolactam-polyvinyl acetate-polyethylene glycol graft co-polymer (e.g., soluplus) is from 1:2 to 1:1 (e.g., from 1:2 to 1:1.1, from 1:2 to 1:1.2, from 1.2 to 1:1.3, from 1:2 to 1:1.4, from 1:2 to 1:1.5, from 1:2 to 1:1.6, from 1:2 to 1:1.7, from 1:2 to 1:1.8, from 1:2 to 1:1.9, from 1:1.9 to 1:1, from 1:1.8 to 1:1, from 1:1.7 to 1:1, from 1:1.6 to 1:1, from 1:1.5 to 1:1, from 1:1.6 to 1:1, from 1:1.5 to 1:1, from 1:1.4 to 1:1, from 1:1.3 to 1:1, from 1:1.2 to 1:1, and from 1:1.1 to 1:1). In some embodiments, the ratio of amorphous silicon dioxide (e.g., Syloid® 244 FP) to polyvinyl caprolactam-polyvinyl acetate-polyethylene glycol graft co-polymer (e.g., soluplus) is 1:1.3.

In some embodiments of the spray-dried dispersion (SDD) formulation, the amorphous solid dispersion of the compound 1, and pharmaceutically acceptable carrier matrix have a weight ratio of the compound 1 to the carrier matrix of from 1:6 to 1:1 (e.g., from 1:6 to 1:2, from 1:6 to 1:2.5, from 1:6 to 1:3, from 1:6 to 1:3.5, from 1:6 to 1:4, from 1:6 to 1:4.5, from 1:6 to 1:5, from 1:5 to 1:2, from 1:5 to 1:2.5, from 1:5 to 1:3, from 1:5 to 1:3.5, from 1:5 to 1:4, from 1:5 to 1:4.5, from 1:5 to 1:1.5, from 1:4 to 1:1.5, from 1:4 to 1:2, from 1:4 to 1:2.5, from 1:4 to 1:3, from 1:4 to 1:3.5, from 1:3 to 1:1.5, from 1:3 to 1:2, from 1:3 to 1:2.5, and from 1:2 to 1:1.5).

In certain embodiments, compound 1 and carrier matrix are present in a ratio of from 1:1 to 1:6 (w/w), 1:1 to 1:4 (w/w), such as in the ratio of 1:2 to 1.3 (w/w).

### 5.1.4. Hot-Melt Extrusion (HME) Formulations

In an embodiment, amorphous solid dispersions of compound 1 are obtained by hot melt extrusion. The term hot-melt extrusion or hot-melt extruded is used herein to describe a process whereby a composition is heated and/or compressed to a molten (or softened) state and subsequently forced through an orifice in a die where the extruded product is formed into its final shape in which it solidifies upon cooling. The blend is conveyed through one or more heating zones typically by a screw mechanism. The screw or screws are rotated by a variable speed motor inside a cylindrical barrel where only a small gap exists between the outside diameter of the screw and the inside diameter of the barrel. In this conformation, high shear is created at the barrel wall and between the screw fights by which the various components of the powder blend are well mixed and disaggregated. The die can be a dual manifold, multi-manifold or feed-block style die.

In some embodiments, there is provided an amorphous solid dispersion comprising compound 1 and a pharmaceutically acceptable carrier matrix, wherein the amorphous solid dispersion is prepared by hot-melt extrusion (e.g., to obtain a hot-melt extrusion (HME) fonnulation). In an embodiment there is provided a spray-dried dispersion comprising 10-30% w/w of compound 1 and 70-90% of a pharmaceutically acceptable carrier matrix, wherein the amorphous solid dispersion is prepared by hot-melt extrusion (e.g., to obtain a hot-melt extrusion (HME) formulation).

Accordingly, in an embodiment there is provided a hot-melt extrusion (HME) formulation comprising compound 1 and a pharmaceutically acceptable carrier matrix. In an embodiment there is provided a hot-melt extrusion formulation comprising 10-30% w/w of compound 1 and 70-90% of a pharmaceutically acceptable carrier matrix.

In some embodiments, the percentage loading of compound 1 in the hot-melt extrusion (HME) formulation is from 15-30% w/w. In some embodiments, the percentage loading of compound 1 in the hot-melt extrusion (HME) formulation is from 20-40% w/w, such as 20%, 21%, 22%, 23%, 24%, 25%, 26%, 27%, 28%, 29%, 30%, 31%, 32%, 33%, 34%, 35%, 36%, 37%, 38%, 39%, or 40% w/w compound 1. In some embodiments, the percentage loading of compound 1 in hot-melt extrusion (HME) formulation is 25% w/w.

In some embodiments, the percentage of the pharmaceutically acceptable carrier matrix in the hot-melt extrusion (HME) formulation is from 60% to 85% (w/w) (e.g., from 70% to 80%, and from 70% to 75%). In some embodiments, the percentage of the pharmaceutically acceptable carrier matrix in the hot-melt extrusion (HME) formulation is from 70% to 75% (w/w), such as 70%, 71%, 72%, 73%, 74%, or 75%. In some embodiments, the percentage of the pharmaceutically acceptable carrier matrix in the hot-melt extrusion (HME) formulation is 75% (w/w).

In some embodiments of the hot-melt extrusion (HME) formulation, the pharmaceutically acceptable carrier matrix comprises a polyvinyl caprolactam-polyvinyl acetate-polyethylene glycol graft co-polymer (e.g., soluplus), a polyvinylpyrrolidone polymer (e.g., PVP K30), a copovidone polymer (e.g., PVP VA64 or Kollidon VA64), a povidone polymer (e.g., Kollidon 17PF), a hydroxypropyl methyl cellulose polymer (e.g., HPMC AS), a dimethylaminoethyl methacrylate-copolymer (e.g., Eudragit EPO), a methacrylic acid-methyl methacrylate copolymer (e.g., Eudragit L100), a polyethylene glycol polymer (e.g., PEG 8000), amorphous silicon dioxide (e.g., Syloid®244 FP) and mixtures thereof.

In some embodiments of the hot-melt extrusion (HME) formulation, the pharmaceutically acceptable carrier matrix comprises a mixture of copovidone polymer, a povidone polymer, and a polyvinyl caprolactam-polyvinyl acetate-polyethylene glycol graft co-polymer.

In some embodiments of the hot-melt extrusion (HME) formulation, the ratio of copovidone polymer, a povidone polymer, and a polyvinyl caprolactam-polyvinyl acetate-polyethylene glycol graft co-polymer is 1:1:1.

In some embodiments of the hot-melt extrusion (HME) formulation, the amorphous solid dispersion of the compound 1, and pharmaceutically acceptable carrier matrix have a weight ratio of the compound 1 to the carrier matrix of from 1:6 to 1:1 (e.g., from 1:6 to 1:2, from 1:6 to 1:2.5, from 1:6 to 1:3, from 1:6 to 1:3.5, from 1:6 to 1:4, from 1:6 to 1:4.5, from 1:6 to 1:5, from 1:5 to 1:2, from 1:5 to 1:2.5, from 1:5 to 1:3, from 1:5 to 1:3.5, from 1:5 to 1:4, from 1:5 to 1:4.5, from 1:5 to 1:1.5, from 1:4 to 1:1.5, from 1:4 to 1:2, from 1:4 to 1:2.5, from 1:4 to 1:3, from 1:4 to 1:3.5, from 1:3 to 1:1.5, from 1:3 to 1:2, from 1:3 to 1:2.5, and from 1:2 to 1:1.5).

In certain embodiments of the hot-melt extrusion (HME) formulation, compound 1 and carrier matrix are present in a ratio of from 1:1 to 1:6 (w/w), 1:1 to 1:4 (w/w), such as in the ratio of 1:2 to 1.3 (w/w).

### 5.2. Pharmaceutical Compositions

As summarized above, this disclosure provides pharmaceutical compositions including the subject amorphous solid dispersion comprising compound 1 or a pharmaceutically acceptable salt thereof, and one or more pharmaceutically acceptable excipients.

The subject amorphous solid dispersion may be used for blending with any one of the excipients described herein (e.g., to form a blended powder or granules) or for filling any one of the dosage forms described herein (e.g., tableting). The amorphous solid dispersion can optionally be further processed before blending or filling. Exemplary further processing includes spheronizing, pelletizing, milling, injection molding, sieving, and/or calendering the solid dispersion.

Amorphous solid dispersions of compound 1 of the present disclosure can be optionally subjected to a particle size reduction procedure before or after the completion of drying of the product to produce desired particle sizes and distributions. Milling or micronization can be performed to achieve the desired particle sizes or distributions. Equipment that may be used for particle size reduction include, without limitation thereto, ball mills, roller mills, hammer mills, and jet mills.

In one embodiment, there is provided an amorphous solid dispersion of compound 1 comprising an amorphous form of compound 1 wherein 90% of the particles are less than about 500 microns or less than about 200 microns or less than about 100 microns or less than about 50 microns or less than about 40 microns or less than about 30 microns or less than about 20 microns or less than about 10 microns or any other suitable particle sizes.

The amorphous solid dispersion of compound 1 may be combined with pharmaceutically acceptable excipients to make other pharmaceutical compositions, or a finished dosage form (e.g., as described herein). The one or more additional pharmaceutically acceptable excipients are selected from diluents, binders, disintegrants, lubricants, glidants, surfactants, solubilizers, plasticizers, stabilizing agents, antioxidants, sweeteners, colors, flavors, preservatives, and combinations thereof.

Other pharmaceutically acceptable excipients may include, but are not limited to, diluents, binders, disintegrating agents, surfactants, plasticizers, lubricants, glidants, chelating agents, coating agents and the like or mixtures thereof as extra-granular agents.

In some embodiments, the one or more pharmaceutically acceptable excipients is selected from diluents, binders, disintegrants, lubricants, glidants, surfactants, solubilizers, plasticizers, stabilizing agents, antioxidants, sweeteners, and any combination thereof.

In some embodiments of the pharmaceutical composition, the percentage of amorphous solid dispersion of compound 1 present is from 1% to 90% (w/w) (e.g., from 1% to 19%, from 10% to 19%, from 10% to 20%, from 10% to 30%, from 10% to 40%, from 10% to 50%, from 10% to 60%, from 10% to 70%, from 10% to 80%, from 10% to 90%, from 20% to 30%, from 20% to 40%, from 20% to 50%, from 20% to 60%, from 20% to 70%, from 20% to 80%, from 20% to 90%, from 21% to 30%, from 21% to 34%, from 21% to 40%, from 21% to 50%, from 21% to 60%, from 21% to 70%, from 21% to 80%, from 21% to 90%, from 30% to 40%, from 30% to 50%, from 30% to 60%, from 30% to 70%, from 30% to 80%, from 30% to 90%, from 36% to 40%, from 36% to 49%, from 36% to 60%, from 36% to 70%, from 36% to 80%, from 36% to 90%, from 40% to 50%, from 40% to 60%, from 40% to 70%, from 40% to 80%, from 40% to 90%, from 50% to 60%, from 50% to 70%, from 50% to 80%, from 50% to 90%, 51% to 60%, from 51% to 70%, from 51% to 80%, from 51% to 90%, from 60% to 70%, from 60% to 80%, from 60% to 90%, from 70% to 80%, and from 70% to 90%). In some embodiments, the percentage of the amorphous solid dispersion in the subject pharmaceutical composition is from 30% to 50% (w/w) (e.g., from 30% to 48%, from 30% to 45%, from 30% to 42%, from 30% to 40%, from 35% to 50%, from 35% to 48%, from 35% to 45%, from 35% to 44%, from 35% to 43%, from 35% to 42%, from 35% to 41%, from 35% to 40%, from 36% to 40%, and from 37% to 40%).

In some embodiments of the pharmaceutical composition, the percentage of amorphous solid dispersion present is from 30% to 50% (w/w), such as 35 to 45% (w/w). In some embodiments of the pharmaceutical composition, the percentage of amorphous solid dispersion present is from 35 to 45%, such as 35%, 36%, 37%, 38%, 39%, 40%, 41%, 42%, 43%, 44%, or 45% (w/w). In some embodiments of the pharmaceutical composition, the percentage of amorphous solid dispersion present is 40% (w/w).

### 5.2.1. Excipients

The pharmaceutical compositions provided in accordance with the present disclosure can be administered orally. In certain embodiments, the disclosure provides pharmaceutical compositions that include a subject amorphous solid dispersion as described herein, and one or more pharmaceutically acceptable excipients or carriers including but not limited to, inert solid diluents and fillers, diluents, including sterile aqueous solution and various organic solvents, permeation enhancers, solubilizers, surfactants, disintegrants, lubricants, binders, glidants, adjuvants, and combinations thereof. Such compositions are prepared in a manner well known in the pharmaceutical art (see, e.g., Remington: The Science and Practice of Pharmacy (Remington: The Science and Practice of Pharmacy, 23rd Edition, ISBN-13: 978-0128200070); and Modern Pharmaceutics, Marcel Dekker, Inc., 4th Ed. (G. S. Banker & C. T. Rhodes, Eds.).

The pharmaceutical compositions may be administered by oral administration. Administration may be via an oral suspension of powder or granules, an oral disintegrating tablet (ODT), or the like. In one embodiment, the subject pharmaceutical composition is in the form of a powder. In one embodiment, the subject pharmaceutical composition is in the fonn of granules. In one embodiment, the subject pharmaceutical composition is in the form of a powder or granules which has been reconstituted as an oral suspension. In one embodiment, the subject pharmaceutical composition is reconstituted in a food or a beverage. In some embodiments, the pharmaceutical composition is in the form of an oral disintegrating tablet (ODT). In making the pharmaceutical compositions that include the solid described herein, the active ingredient is usually diluted by an excipient and/or enclosed within such a carrier that can be in the form of a sachet, or other container. When the excipient serves as a diluent, it can be in the form of a solid, semi-solid or liquid material (as above), which acts as a vehicle, carrier or medium for the active ingredient.

The pharmaceutical composition may be formulated for immediate release or sustained release. A “sustained release formulation” is a formulation which is designed to slowly release a therapeutic agent in the body over an extended period of time, whereas an “immediate release formulation” is a formulation which is designed to quickly release a therapeutic agent in the body over a shortened period of time. In some cases, the immediate release formulation may be coated such that the therapeutic agent is only released once it reached the desired target in the body (e.g., the stomach). In a specific embodiment, the pharmaceutical composition is formulated for immediate release.

The pharmaceutical composition may further comprise pharmaceutical excipients such as fillers or diluents, binders, glidants, disintegrants, lubricants, solubilizers, and combinations thereof. Some examples of suitable excipients are described herein. When the pharmaceutical composition is formulated into a tablet, the tablet may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In some embodiments, the pharmaceutical composition comprises a diluent or a filler, such as a carbohydrate or a protein filler. In some embodiments, the diluent is selected from the group consisting of dicalcium phosphate, cellulose, microcrystalline cellulose, hydroxypropylmethyl cellulose, sodium carboxymethylcellulose, compressible sugars, dibasic calcium phosphate dehydrate, lactose, lactose monohydrate, sucrose, mannitol, sorbital, starch from corn, wheat, rice, potato or other plants, tribasic calcium phosphate, a gum (e.g., Arabic or tragacanth), proteins (e.g., gelatin or collagen) and combinations thereof. In some embodiments of the pharmaceutical composition, the diluent is selected from microcrystalline cellulose, dicalcium phosphate, cellulose, compressible sugars, dibasic calcium phosphate dehydrate, lactose, lactose monohydrate, lactose anhydrous, mannitol, tribasic calcium phosphate, a partially pregelatinized starch and combinations thereof.

In further embodiments, the pharmaceutical composition comprises one or more diluents in an amount from 10 to 70% w/w, or from 20 to 70% w/w, or from 25% to 70% w/w, or from 30 to 70% w/w, or from 35 to 70% w/w, or from 40 to 70% w/w, or from 45 to 65% w/w, or from 45 to 60% w/w, or from 50 to 60% w/w. In some embodiments, one or more diluents is present in an amount of 50 to 60% w/w, such as 50%, 51%, 52%, 53%, 54%, 55%, 56%, 57%, 58%, 59% or 60% w/w. In some embodiments, one or more diluents is present in an amount of 50 to 51% w/w. In some embodiments, one or more diluents is present in an amount of 58 to 59% w/w.

In some embodiments of the pharmaceutical composition, the diluent is microcrystalline cellulose present in an amount of from 50 to 60% w/w, such as 50 to 51% w/w, 51 to 52% w/w, 52 to 53% w/w, 53 to 54% w/w, 54 to 55% w/w, 55 to 56% w/w, 56 to 57% w/w, 57 to 58% w/w, 58 to 59% w/w, or 59 to 60% w/w. In some embodiments of the pharmaceutical composition, the diluent is mannitol present in an amount of 50 to 60% w/w, such as 50 to 51% w/w, 51 to 52% w/w, 52 to 53% w/w, 53 to 54% w/w, 54 to 55% w/w, 55 to 56% w/w, 56 to 57% w/w, 57 to 58% w/w, 58 to 59% w/w, or 59 to 60% w/w. In some embodiments of the pharmaceutical composition, the diluent is a pregelatinized starch (e.g., starch 1,500) present in an amount of 50 to 60% w/w, such as 50 to 51% w/w, 51 to 52% w/w, 52 to 53% w/w, 53 to 54% w/w, 54 to 55% w/w, 55 to 56% w/w, 56 to 57% w/w, 57 to 58% w/w, 58 to 59% w/w, or 59 to 60% w/w.

In some embodiments of the pharmaceutical composition, the diluent used has a combination of properties. For example, in some cases the diluent can also acts as a binder and/or a disintegrant.

In some embodiments, the pharmaceutical composition comprises a disintegrant or solubilizing agent selected from cross-linked polyvinyl pyrrolidone, agar, alginic acid or a salt thereof (e.g., sodium alginate), croscarmellose sodium, crospovidone, modified corn starch, pregelatinized starch, sodium starch glycolate, and combinations thereof.

In certain embodiments, the pharmaceutical composition comprises one or more disintegrants in an amount from 1 to 10% w/w, or from 1 to 9% w/w, or from 1 to 8% w/w, or from 2 to 8% w/w, or from 4 to 6% w/w. In some embodiments, one or more disintegrants is present in an amount of 1%, 2%, 3%, 4%, 5%, 6%, 7%, 8%, 9%, or 10% w/w. In some embodiments, the disintegrant is croscarmellose sodium present in an amount of 4 to 6% w/w. In a further specific embodiment, the disintegrant is croscarmellose sodium present in an amount of 5% w/w.

In some embodiments, the pharmaceutical composition comprises a lubricant selected from the group consisting of calcium stearate, magnesium stearate, polyethylene glycol, sodium stearyl fumarate, stearic acid, and combinations thereof.

In some embodiments, the pharmaceutical composition comprises one or more lubricants in an amount from about 0.1 to about 2% w/w, or from about 0.5 to about 1.8% w/w, or from about 0.5 to about 1.5% w/w, or from about 0.5 to about 1.4% w/w, or from about 0.5 to about 1.3% w/w, or from about 0.5 to about 1.2% w/w, or from about 0.5 to about 1.1% w/w. In some embodiments, the lubricant is sodium stearyl fumarate present in an amount of 0.5%, 0.6% w/w, 0.7% w/w, 0.8% w/w, 0.9% w/w, or 1.0% w/w. In some embodiments, the lubricant is sodium stearyl fumarate present in an amount of 1.0% w/w.

In some embodiments, the pharmaceutical composition comprises a glidant selected from the group consisting of colloidal silicon dioxide, talc, and combinations thereof.

In some embodiments, the pharmaceutical composition comprises one or more glidants in an amount from about 0.1 to about 2.5% w/w, or from about 0.5 to about 2.0% w/w, or from about 0.5 to about 1.5% w/w. In specific embodiments, one or more glidants is present in an amount of 0.5%, 0.6%, 0.7%, 0.8%, 0.9%, 1.0%, 1.1%, 1.2%, 1.3%, 1.4%, or 1.5% w/w. In some embodiments, the glidant is colloidal silicon dioxide present in an amount of 1.5% w/w.

In some embodiments, the pharmaceutical composition comprises a sweetener. In some embodiments, the sweetener is an artificial sweetener, such as sucralose. It will be understood than any convenient sweetener can be used in the present disclosure.

In some embodiments, the pharmaceutical composition comprises one or more sweeteners in an amount from 1 to 10% w/w, or from 1 to 9% w/w, or from 1 to 8% w/w, or from 2 to 8% w/w, or from 4 to 6% w/w, or from 1 to 5% w/w, or from 1 to 2.5% w/w. In some embodiments, one or more sweeteners is present in an amount of 1%, 2%, 3%, 4%, 5%, 6%, 7%, 8%, 9%, or 10% w/w. In some embodiments, the sweetener is sucralose present in an amount of from 1.5 to 2.5% w/w. In some embodiments, the sweetener is sucralose present in an amount of from 2 to 3% w/w.

In some embodiments, the pharmaceutical composition comprises one or more surfactants. Suitable surfactants include both non-ionic and ionic (cationic, anionic and zwitterionic) surfactants suitable for use in pharmaceutical dosage forms. These include polyethoxylated fatty acids and its derivatives, for example, polyethylene glycol 400 distearate, polyethylene glycol-20 dioleate, polyethylene glycol 4-150 mono dilaurate, and polyethylene glycol—20 glyceryl stearate; alcohol—oil transesterification products, for example, polyethylene glycol—6 corn oil; polyglycerized fatty acids, for example, polyglyceryl—6 pentaoleate; propylene glycol faty acid esters, for example, propylene glycol monocaprylate; mono and diglycerides, for example, glyceryl ricinoleate; sterol and sterol derivatives; sorbitan fatty acid esters and its derivatives, for example, polyethylene glycol—20 sorbitan monooleate and sorbitan monolaurate; polyethylene glycol alkyl ether or phenols, for example, polyethylene glycol—20 cetyl ether and polyethylene glycol—10-100 nonyl phenol; sugar esters, for example, sucrose monopalmitate; polyoxyethylene-polyoxypropylene block copolymers known as “poloxamer;” ionic surfactants, for example, sodium caproate, sodium glycocholate, soy lecithin, sodium stearyl fumarate, propylene glycol alginate, octyl sulfosuccinate disodium, and palmitoyl carnitine; and the like and mixtures thereof. The concentration of surfactant ranges from about 0.5% to about 10% w/w of total composition.

In some embodiments, the pharmaceutical composition may include one or more plasticizers. Suitable plasticizers include polyethylene glycol, propylene glycol, polyethylene oxide, 1,2-butylene glycol, 2,3-butylene glycol, styrene glycol, diethylene glycol, triethylene glycol, tetraethylene glycol and monoisopropyl ether, propylene glycol monoethyl ether, ethylene glycol monoethyl ether, diethylene glycol monoethyl ether, sorbitol lactate, ethyl lactate, butyl lactate, ethyl glycolate, triethyl citrate, acetyl triethyl citrate, tributyl citrate and allyl glycolate. The concentration of plasticizer ranges from about 0.5% to about 10% w/w of total composition.

In some embodiments, the pharmaceutical composition may include a coloring agent. Suitable coloring agents include dyes and pigments such as iron oxide red or yellow, titanium dioxide, talc. The concentration of coloring agent can range from about 0.1% to about 1% w/w of the total composition.

In some embodiments, the pharmaceutical composition may include a chelating agent. Suitable chelating agents include, one or more of, but not limited to ethylenediaminetetraacetic acid (EDTA), disodium EDTA and derivatives thereof, citric acid and derivatives thereof, niacinamide and derivatives thereof, and sodium desoxycholate and the like or mixtures thereof. The concentration of chelating agent can range from about 0.1% to about 1% w/w of total composition.

In some embodiments, the pharmaceutical composition comprises the amorphous solid dispersion (e.g., as described herein), one or more diluents, one or more disintegrants, and one or more additional actives. In some embodiments, the pharmaceutical composition comprises 30-50% w/w of the amorphous solid dispersion, 40-70% w/w of one or more diluents, 1-10% w/w of one or more disintegrants, and up to 100% w/w of one or more additional additives. In some embodiments, the pharmaceutical composition comprises 35-45% w/w of the amorphous solid dispersion, 50-60% w/w of one or more diluents, 4-6% w/w of one or more disintegrants, and up to 100% w/w of one or more additional additives. In some embodiments, one or more additional additives includes a lubricant, a glidant and a sweetener.

In some embodiments, the pharmaceutical composition comprises:

| Component | % w/w |
|---|---|
| the amorphous solid dispersion (e.g., as | 30 to 50% |
| described herein) | |
| a diluent | 40 to 70% |
| a disintegrant | 1 to 10% |
| a lubricant | 0.5 to 2% |
| a glidant | 0.5 to 2% |
| a sweetener | 0 to 5% |

In some embodiments, the pharmaceutical composition comprises:

| Component | % w/w |
|---|---|
| the amorphous solid dispersion (e.g., as | 35 to 45% |
| described herein) | |
| a diluent | 50 to 60% |
| a disintegrant | 4 to 6% |
| a lubricant | 0.5 to 1.5% |
| a glidant | 1 to 2% |
| a sweetener | 0 to 5% |

In some embodiments, the pharmaceutical composition includes 1 to 5% of a sweetener. In some embodiments, the sweetener is an artificial sweetener. In some embodiments, the sweetener is sucralose.

The pharmaceutical compositions described herein can be formulated with an amorphous solid dispersion of compound 1 (e.g., as described herein) as the sole pharmaceutically active ingredient in the composition or can be combined with other active ingredients.

In certain embodiments, the pharmaceutical composition is formulated into one or more suitable pharmaceutical preparations, such as oral suspensions, powders, granules, an oral disintegrating tablet (ODT), sustained release formulations or elixirs in sterile solutions or suspensions for parenteral administration, or as transdermal patch preparation and dry powder inhalers.

Concentrations of the compound 1 in a pharmaceutical composition provided herein will depend on, e.g., the physicochemical characteristics of the compound, the dosage schedule, and amount administered as well as other factors known to those of skill in the art. For example, if the composition comprises a salt of compound 1 the amount of said salt to be administered and/or to be incorporated into a pharmaceutical composition (i.e., pharmaceutical dosage form) needs to be adjusted to take account of the molecular weight difference between the free base and salt form. For instance, in expressing dose amounts in the label and/or product information of authorized medicinal products comprising a salt form of an active compound that can also be used in free base form, it is customary practice to specify the dose of the free base to which the dose of the salt as used is equivalent.

Pharmaceutical compositions described herein are provided for administration to a subject, for example, humans or animals (e.g., mammals) in unit dosage forms, such as sterile parenteral (e.g., intravenous) suspensions containing suitable quantities of the compounds or pharmaceutically acceptable derivatives thereof. Pharmaceutical compositions are also provided for administration to humans and animals in unit dosage form, including powders, granules, oral or nasal solutions or suspensions and oil-water emulsions containing suitable quantities of compound 1 or pharmaceutically acceptable derivatives thereof. The amorphous solid dispersion of compound 1 is, in certain embodiments, formulated and administered in unit-dosage forms or multiple-dosage forms. Unit-dose forms as used herein refers to physically discrete units suitable for human or animal (e.g., mammal) subjects and packaged individually as is known in the art. Each unit-dose contains a predetermined quantity of compound 1 sufficient to produce the desired therapeutic effect, in association with the required pharmaceutical carrier, vehicle or filler. Examples of unit-dose forms include sachets, ampoules and syringes and individually packaged tablets. Unit-dose forms can be administered in fractions or multiples thereof. A multiple-dose form is a plurality of identical unit-dosage forms packaged in a single container to be administered in segregated unit-dose form. Examples of multiple-dose forms include sachets, vials, or bottles. Hence, in specific aspects, multiple dose form is a multiple of unit-doses which are not segregated in packaging.

In some embodiments the subject pharmaceutical composition is formulated as powders, granules, tablets, pills, dragees, capsules, liquids, gels, syrups, slurries, suspensions, and the like, suitable for ingestion by a subject. In certain cases, the pharmaceutical composition is formulated as a dragee, and dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the dragee coatings for product identification or to characterize the quantity of active compound (i.e., dosage).

In some embodiments, the subject pharmaceutical composition is formulated for oral use as push-fit capsules made of gelatin, as well as soft, sealed capsules made of gelatin having a coating such as glycerol or sorbitol. Push-fit capsules can contain an amorphous solid dispersion of compound 1 mixed excipients as described herein, e.g., diluents or binders such as microcrystalline cellulose, lubricants such as talc or magnesium stearate, and optionally stabilizers. In soft capsules, the amorphous solid dispersion of compound 1 may be suspended in suitable liquids, such as fatty oils, liquid paraffin, or liquid polyethylene glycol with or without stabilizers.

In some embodiments, the subject pharmaceutical composition is formulated for oral use as a powder or granules, the unit doses of which can be individually packaged in sachets. The powder or granules can contain an amorphous solid dispersion of compound 1 mixed excipients as described herein, e.g., diluents such as microcrystalline cellulose, disintegrants such as croscarmellose sodium, lubricants such as sodium stearyl fumarate, and, optionally one or more other excipients, such as glidants and sweeteners.

In certain embodiments, the amorphous solid dispersion of compound 1 described herein are in a liquid pharmaceutical formulation. Liquid pharmaceutically administrable formulations can, for example, be prepared by dispersing, or otherwise mixing the active compounds and optional pharmaceutical adjuvants in a carrier, such as, for example, water, saline, aqueous dextrose, glycerol, glycols, and the like, to thereby form a solution or suspension. In certain embodiments, a pharmaceutical composition provided herein to be administered can also contain minor amounts of nontoxic auxiliary substances such as wetting agents, emulsifying agents, solubilizing agents, and pH buffering agents and the like.

Actual methods of preparing such dosage forms are known, or will be apparent, to those skilled in this art; for example, see, e.g., Remington: The Science and Practice of Pharmacy (Remington: The Science and Practice of Pharmacy, 23rd Edition, ISBN-13: 978-0128200070) Dosage forms or compositions containing compound 1 in the ranges disclosed herein with the balance made up from non-toxic carrier can be prepared.

In certain embodiments, the pharmaceutical formulations are powders or granules, which can be reconstituted for administration as suspensions, emulsions and other mixtures. They can also be reconstituted and formulated as solids or gels.

In certain embodiments, the pharmaceutical composition is formulated in as a solid dosage form, such as an oral disintegrating tablet, a tablet or a capsule.

In some embodiments, the disclosure relates to pharmaceutical compositions comprising amorphous solid dispersions of compound 1 for oral administration, which solid dispersion comprises at least one carrier polymer.

In some embodiments, the present disclosure relates to pharmaceuticals compositions as described herein, which are stable, e.g., stable over the shelf life of the drug product. As used herein, the term “stable” is defined as no more than about 5% loss of compound 1 under typical commercial storage conditions. In certain embodiments, the formulations of the present invention will have no more than about 3% loss of compound 1, such as, no more than about 2% loss of compound 1, under typical commercial storage conditions. The composition retains at least about 95% of the potency of compound 1 after storing the composition at 40° C. and 75% relative humidity for at least three months. In certain aspects, the term “stable” refers to chemical stability, wherein not more than 1.5% w/w of total related substances are formed on storage at accelerated conditions of stability at 40° C. and 75% relative humidity or at 25° C. and 60% relative humidity for a period of at least three months or to the extent necessary for use of the composition.

### 5.3. Pharmaceutical Dosage forms

As summarized above, this disclosure provides a pharmaceutical dosage form comprising the pharmaceutical composition described herein. The disclosure provides for powders, granules, oral disintegrating tablets, tablets, pills, capsules, oral suspensions and the like, comprising the pharmaceutical compositions or dosage forms described herein. Accordingly, in some embodiments, the pharmaceutical dosage from is a solid dosage form. In certain cases, the solid dosage form is a powder. In certain cases, the capsule is in the form of granules. In certain embodiments, the solid dosage form is in the form of a power or granules packaged in individually labelled sachets containing a single dose. In certain cases, the dosage form is an oral disintegrating tablet (ODT).

In an embodiment, pharmaceutical compositions comprising amorphous solid dispersion of compound 1 and pharmaceutically acceptable excipients are prepared by using, wet granulation, dry granulation, or direct compression.

In an embodiment, pharmaceutical compositions comprising amorphous solid dispersion of compound 1 and pharmaceutically acceptable excipients are prepared by using direct compression, which process comprises mixing amorphous solid dispersion of compound 1 and pharmaceutically acceptable excipients, the resultant mixture is either compressed to a tablet (e.g., an oral disintegrating tablet (ODT)), or filled in hard gelatin capsules.

In an embodiment, pharmaceutical compositions comprising amorphous solid dispersion of compound 1 and pharmaceutically acceptable excipients are prepared by using dry granulation, wherein dry granulation is carried out by either direct compaction or roller compaction or both.

In an embodiment, pharmaceutical compositions comprising amorphous solid dispersion of compound 1 and pharmaceutically acceptable excipients are prepared by using direct compaction dry granulation, which process comprises compressing mixture of amorphous solid dispersion of compound 1 and excipients into slugs, compressed slugs are milled and passed through mess screen manually or automatically which results in granules. The resulting granules can be either filled into sachets, or compressed to tablet (e.g., an oral disintegrating tablet (ODT)), and the like.

In an embodiment, pharmaceutical compositions comprising amorphous solid dispersion of compound 1 and pharmaceutically acceptable excipients are prepared by using roller compaction dry granulation, which process comprises passing a mixture of amorphous solid dispersion of compound 1 and excipients between two high-pressure rollers to form consolidated and densified material, the resultant densified material is then reduced to a uniform granule size by milling, which can then can be either filled into sachets, or compressed to tablet (e.g., an oral disintegrating tablet (ODT)), and the like.

In an embodiment, pharmaceutical compositions comprising amorphous solid dispersion of compound 1 and pharmaceutically acceptable excipients are prepared by wet granulation, which process comprises: (a) mixing amorphous solid dispersion of compound 1 and pharmaceutically acceptable excipients (b) adding sufficient solvent to the mixture obtained from step (a) under shear to generate granules; (c) milling or grinding the granules followed by sieving of said granules; optionally mixing with other excipients. The resulting granules can be either filled into sachets, or compressed to tablet (e.g., an oral disintegrating tablet (ODT)), and the like.

The disclosure also relates to pharmaceutical dosage forms comprising amorphous solid dispersion of compound 1 for oral administration, which solid dispersion further comprises at least one pharmaceutically acceptable carrier (e.g., a carrier polymer). In some embodiments, the dosage form is formulated for immediate-release. In some embodiments, the dosage form is formulated for modified-release portion.

As used herein the term “immediate-release” refers to the rapid release of the majority of the therapeutic compound. Particularly useful conditions for immediate-release are release of at least or equal to about 80% of the therapeutic compound within thirty minutes after oral ingestion. The particular immediate-release conditions for a specific therapeutic compound will be recognized or known by one of ordinary skill in the art.

As used herein the term “modified-release” refers to slower release of the majority of the therapeutic compound as compared to immediate release dosage forms. The particular modified-release conditions for a specific therapeutic compound will be recognized or known by one of ordinary skill in the art.

The pharmaceutical compositions are manufactured by processes such as direct compression, wet granulation or dry granulation. The pharmaceutical compositions are in the form of oral dosage forms, such as solid oral dosage forms, including powders, granules, oral disintegrating tablets (ODT), oral suspensions, and multi-particulates.

In some embodiments, the pharmaceutical composition of the present disclosure is a granulate/particulate material. The granules/particles may be filled into a sachet or compressed into a tablet. The tablet may optionally be coated with an additional enteric polymer or an immediate-release coating.

Moreover, the extrudates/granules of the present disclosure may be formulated into any suitable dosage form, including but not limited to oral suspensions, gels, oral disintegrating tablets (ODTs), tablets, capsules, immediate release formulations, delayed release formulations, controlled release formulations, extended-release formulations, pulsatile release formulations, and mixed immediate and controlled release formulations.

The tablets or pills of the present disclosure may be coated to provide a dosage form affording the advantage of prolonged action or to protect from the acid conditions of the stomach. The tablets may also be formulated for immediate release. In certain embodiments, the tablet comprises a film coating. A film coating may be useful for limiting photolytic degradation. Suitable film coatings are selected by routine screening of commercially available preparations. In one embodiment, the film coating is a hypromellose-based coating. In certain embodiments, the coating comprises a film-forming agent, a plasticizer, a glidant and optionally one or more pigments. An exemplary film coating composition may comprise hydroxypropyl methylcellulose (HPMC), lactose monohydrate, titanium dioxide, and triglyceride 1,2,3-triacetoxypropane (triacetin). In certain cases, the film coating composition may comprise hydroxypropyl methylcellulose (HPMC), polyethylene glycol (PEG), talc, titanium dioxide and optionally iron oxide, including iron oxide red and/or yellow.

In some embodiments, the pharmaceutical dosage form comprises compound 1 in a therapeutically effective amount (e.g., as described herein for compound 1).

In some embodiments, the pharmaceutical dosage form comprises from 10-5000 mg of compound 1. In some embodiments, the pharmaceutical dosage form comprises 10 mg, 15 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 450 mg, 500 mg, 550 mg, 600 mg, 650 mg, 700 mg, 750 mg, 800 mg, 850 mg, 900 mg, 950 mg, or 1000 mg+/−10% of compound 1. In certain embodiments, the pharmaceutical dosage form comprises 1500 mg, 2000 mg, 2500 mg, 3000 mg, 3500 mg, 4000 mg, 4500 mg, or 5000 mg+/−10% of compound 1.

In some embodiments, the pharmaceutical dosage form comprises 25-2000 mg of compound 1. In some embodiments, the pharmaceutical dosage form comprises 25 mg, 50 mg, 75 mg, 100 mg, 125 mg, 150 mg, 175 mg, 200 mg, 225 mg, 250 mg, 275 mg, 300 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 900 mg, 925 mg, 950 mg, 975 mg, or 1000 mg of compound 1.

In some embodiments, the pharmaceutical dosage form comprises from 50 mg to 450 mg compound 1. In some embodiments, the pharmaceutical dosage form comprises from 50 mg to 100 mg compound 1, or from 100 mg to 150 mg compound 1, or from 150 mg to 200 mg compound 1, or from 250 mg to 300 mg compound 1, or from 300 mg to 350 mg compound 1, from 350 mg to 400 mg, or 400 mg to 450 mg of compound 1. In some embodiments, the pharmaceutical dosage form comprises 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg+/−10% of compound 1. In some embodiments, the pharmaceutical dosage form comprises 210 mg, 220 mg, 230, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, or 450 mg+/−10% of compound 1. In an embodiment, the pharmaceutical dosage form comprises 50 mg+/−10% of compound 1. In an embodiment, the pharmaceutical dosage form comprises 100 mg+/−10% of compound 1. In some embodiments, the pharmaceutical dosage form comprises 200 mg+/−10% of compound 1. In some embodiments, the pharmaceutical dosage form comprises 300 mg+/−10% of compound 1. In some embodiments, the pharmaceutical dosage form comprises 400 mg+/−10% of compound 1. In some embodiments, the pharmaceutical dosage form comprises 450 mg+/−10% of compound 1.

In certain embodiments, the pharmaceutical dosage form comprises one or more excipients (e.g., as described herein). In certain embodiments, the pharmaceutical dosage form comprises one or more diluents. In certain embodiments, the pharmaceutical dosage from comprises microcrystalline cellulose.

In certain embodiments, the pharmaceutical dosage form comprises one or more diluents (e.g., microcrystalline cellulose) in an amount from 300 mg to 700 mg, such as 350 mg to 650 mg, 400 mg to 650 mg, 450 mg to 650 mg, or 500 to 600 mg. In some embodiment, the diluent is present in an amount of 500 mg, 510 mg, 520 mg, 530 mg, 540 mg, 550 mg, 560 mg, 570 mg, 580 mg, 590 mg, or 600 mg+/−10%.

In certain embodiments, the pharmaceutical dosage form comprises microcrystalline cellulose in an amount from 300 mg to 700 mg, such as 350 mg to 650 mg, 400 mg to 650 mg, 450 mg to 650 mg, or 500 to 600 mg. In a specific embodiment, the microcrystalline cellulose is in an amount of 500 mg, 510 mg, 520 mg, 530 mg, 540 mg, 550 mg, 560 mg, 570 mg, 580 mg, 590 mg, or 600 mg+/−10%.

In certain embodiments, the pharmaceutical dosage form comprises one or more disintegrants. In certain cases, the disintegrant is croscarmellose sodium. In certain embodiments, the pharmaceutical dosage form comprises a disintegrant in an amount from 15 mg to 65 mg, such as about 15 mg to 60 mg, 20 mg to 60 mg, 25 mg to 60 mg, 30 mg to 60 mg, 35 to 55 mg, or about 40 to 50 mg. In certain embodiments, the pharmaceutical dosage from comprises a disintegrant in an amount of from 45 mg to 55 mg, such as 45 mg, 46 mg, 47 mg, 48 mg, 49 mg, 50 mg, 51 mg, 52 mg, 53 mg, 54 mg, or 55 mg+/−10%. In some embodiments, the disintegrant is in an amount of 50 mg.

In certain embodiments, the pharmaceutical dosage form comprises croscarmellose sodium in an amount from 15 mg to 65 mg, such as about 15 mg to 60 mg, 20 mg to 60 mg, 25 mg to 60 mg, 30 mg to 60 mg, 35 to 55 mg, or about 40 to 50 mg. In certain embodiments, the pharmaceutical dosage from comprises croscarmellose sodium in an amount of from 45 mg to 55 mg, such as 45 mg, 46 mg, 47 mg, 48 mg, 49 mg, 50 mg, 51 mg, 52 mg, 53 mg, 54 mg, or 55 mg+/−10%. In an embodiment, the croscarmellose sodium is in an amount of 50 mg.

In certain embodiments, the pharmaceutical dosage form comprises one or more lubricants. In certain cases, the lubricant is sodium stearyl fumarate. In certain embodiments, the pharmaceutical dosage form comprises a lubricant in an amount from 1 mg to 15 mg, such as 2 to 12 mg, 2 to 10 mg, 3 to 10 mg, 4 to 10 mg, or 5 to 10 mg. In certain embodiments, the pharmaceutical dosage from comprises a lubricant in an amount from 5 mg to 10 mg, such as 5 mg, 6 mg, 7 mg, 8 mg, 9 mg, or 10 mg+/1 10%. In a specific embodiment, the lubricant is present in an amount of 10 mg.

In certain embodiments, the pharmaceutical dosage form comprises sodium stearyl fumarate in an amount from 1 mg to 15 mg, such as 2 to 12 mg, 2 to 10 mg, 3 to 10 mg, 4 to 10 mg, or 5 to 10 mg. In certain embodiments, the pharmaceutical dosage from comprises sodium stearyl fumarate in an amount from 5 mg to 10 mg, such as 5 mg, 6 mg, 7 mg, 8 mg, 9 mg, or 10 mg+/1 10%. In a specific embodiment, the sodium stearyl fumarate is in an amount of 10 mg.

In some embodiments, the pharmaceutical dosage form comprises one or more glidants. In some cases, the glidant is colloidal silicon dioxide. In some embodiments, the pharmaceutical dosage form comprises a glidant in an amount from 5 mg to 25 mg, such as about 10 to 25 mg, 10 to 20 mg, or 15 to 20 mg. In some embodiments, the pharmaceutical dosage from comprises a glidant in an amount from 15 to 20 mg, such as 15 mg, 16 mg, 17 mg, 18 mg, 19 mg, or 20 mg+/−10%. In an embodiment, the glidant is present in an amount of 15 mg. In an embodiment, the glidant is present in an amount of 20 mg.

In some embodiments, the pharmaceutical dosage form comprises colloidal silicon dioxide in an amount from 5 mg to 25 mg, such as about 10 to 25 mg, 10 to 20 mg, or 15 to 20 mg. In some embodiments, the pharmaceutical dosage form comprises colloidal silicon dioxide in an amount from 15 to 20 mg, such as 15 mg, 16 mg, 17 mg, 18 mg, 19 mg, or 20 mg+/−10%. In an embodiment, the colloidal silicon dioxide is present in an amount of 15 mg. In an embodiment, the colloidal silicon dioxide is present in an amount of 20 mg.

In some embodiments, the pharmaceutical dosage form comprises one or more sweeteners. In some cases, the sweetener is sucralose. In some embodiments, the pharmaceutical dosage form comprises a sweetener in an amount from 1 mg to 30 mg, such as about 1 to 25 mg, 1 to 23 mg, 5 to 25 mg, 10 to 25 mg, 15 to 25 mg, or 20 to 25 mg. In some embodiments, the pharmaceutical dosage from comprises a sweetener in an amount from 20 to 25 mg, such as 20 mg, 21 mg, 22 mg, 23 mg, 24 mg, or 25 mg+/−10%. In an embodiment, the sweetener is present in an amount of 22-23 mg.

In some embodiments, the pharmaceutical dosage form comprises sucralose in an amount from 1 mg to 30 mg, such as about 1 to 25 mg, 1 to 23 mg, 5 to 25 mg, 10 to 25 mg, 15 to 25 mg, or 20 to 25 mg. In some embodiments, the pharmaceutical dosage form comprises sucralose in an amount from 20 to 25 mg, such as 20 mg, 21 mg, 22 mg, 23 mg, 24 mg, or 25 mg+/−10%. In an embodiment, the colloidal silicon dioxide is present in an amount of 15 mg. In an embodiment, the sucralose is present in an amount of 22-23 mg.

In some embodiments, the pharmaceutical dosage form comprises a) 50-400 mg of an amorphous solid dispersion of compound 1 (e.g., as described herein); b) 300-700 mg of a diluent; c) 15-65 mg of a disintegrant; d) 1-15 mg of a lubricant; e) 5-25 mg of a glidant; and f) optionally 1-25 mg of a sweetener.

In some embodiments, the pharmaceutical dosage form comprises a) 50-400 mg of an amorphous solid dispersion of compound 1 (e.g., as described herein); b) 300-700 mg of microcrystalline cellulose; c) 15-65 mg of croscarmellose sodium; d) 1-15 mg of sodium stearyl fumarate; e) 5-25 mg of colloidal silicon dioxide; and f) optionally 1-25 mg of sucralose.

In some embodiments, the subject pharmaceutical dosage form (e.g., as described herein) may be administered orally. In certain cases, the pharmaceutical dosage form (e.g., as described herein in Example 4) is administered to a human subject to produce: a maximum blood plasma concentration (average Cₘₐₓ) that is greater than that achieved with a control formulation of compound 1 (e.g., as described herein); and/or an area under the curve (AUC) for compound 1 and/or a metabolite of compound 1 that is greater than that achieved with a control formulation of compound 1. In certain cases, the metabolite of compound 1 is 2-(4-(tert-butyl)phenyl)-1H-benzo[d]imidazol-5-ol (compound 2):

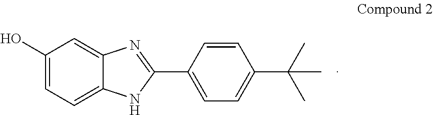 [SMILES: CC(C)(C)c1ccc(-c2nc3cc(O)ccc3[nH]2)cc1]

In some embodiments, the pharmaceutical composition, or pharmaceutical dosage form as described herein is free of negative drug-drug interactions. In a related embodiment, the pharmaceutical composition, or pharmaceutical dosage form is free of negative drug-drug interactions with other active agents. In a further embodiment, the pharmaceutical composition, or pharmaceutical dosage form as described herein is administrable without regard to food and with or without regard to the patient being on another therapeutic agent.

### 5.4. Kits

Also provided herein is a pharmaceutical kit comprising a pharmaceutical dosage form, or a plurality of unit pharmaceutical dosage forms (e.g., as described herein) and instructions for use. In some embodiments, the kit includes a pharmaceutical dosage form, or a plurality of unit pharmaceutical dosage forms (e.g., as described herein) and instructions for oral administration of the dosage forms, wherein the instructions indicate that the composition can be reconstituted in a food or beverage.

In accordance with embodiments of the invention, the pharmaceutical kit comprises a container, such as a box including one or more blister packs, or one or more sachets, wherein the sachets or blister packs can contain a plurality of solid unit pharmaceutical dosage forms as described herein. In certain embodiments, the container or pack comprises at least 5, at least 8, at least 10, at least 12 of at least 15 of said unit pharmaceutical dosage forms, e.g., 4, 5, 6, 7, 8, 9, 10, 11, 12, 13, 14, 15, 16, 17, 18, 19 or 20 of said unit dosage forms.

In accordance with the disclosure, the pharmaceutical kit comprises instructions (e.g., a leaflet) inserted into the container or box, typically a patient information leaflet containing printed information, which information may include a description of the form and composition of the unit pharmaceutical dosage forms contained in the kit, an indication of the therapeutic indications for which the product is intended, instructions as to how the product is to be used and information and warnings concerning adverse effects and contraindications associated with the use. In accordance with the present disclosure, the leaflet will usually contain the information concerning the therapeutic indications, uses, treatment regimens, etc. as described herein in relation to the methods of treatment of the present invention. In certain cases, the leaflet contains printed instructions to repeatedly (self-)administer the pharmaceutical unit dosage forms in order to treat and/or prevent a disease or condition, such as a neurodegenerative disease, including Parkinson's disease, Alzheimer's disease, and amyotrophic lateral sclerosis (ALS).

### 5.5. Methods of Use

As summarized above, also provided herein are methods of delivering the subject pharmaceutical dosage forms to a subject to achieve an enhanced maximum blood plasma concentration (Cₘₐₓ) for compound 1 that is greater than that achieved with a control formulation of compound 1 (e.g., as described herein in Example 4), and/or an area under the curve (AUC) for compound 1 and/or a metabolite of compound 1 (e.g., compound 2) that is greater than that achieved with a control formulation of compound 1. In certain cases, the metabolite of compound 1 is 2-(4-(tert-butyl)phenyl)-1H-benzo[d]imidazol-5-ol (compound 2).

An exemplary dosage may be a powder, granules (e.g., powder or granules reconstituted in a food or beverage for oral administration), an oral suspension, or an oral disintegrating tablet, taken from one to six times daily. In certain embodiments, the pharmaceutical dosage form is administered once daily. In certain embodiments, the pharmaceutical dosage form is administered twice daily. In some embodiments, the pharmaceutical dosage form is administered three times daily. In some embodiments, the pharmaceutical dosage form is administered four times daily. In some embodiments, the pharmaceutical dosage form is administered five times daily. In some embodiments, the pharmaceutical dosage form is administered six times daily.

In some embodiments, multiple doses of the pharmaceutical dosage form are administered. The frequency of administration of the dosage form can vary depending on any of a variety of factors, e.g., severity of the symptoms, etc. For example, in some embodiments, the subject dosage form is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid). In certain embodiments, the subject dosage form is administered twice a day (bid).

The duration of administration of a pharmaceutical dosage from, e.g., the period of time over which compound 1 is administered, can vary, depending on any of a variety of factors, e.g., patient response, etc. For example, an active agent can be administered over a period of time ranging from about one day to about one week, from about two weeks to about four weeks, from about one month to about two months, or from about two months to about four months, or more.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

Although the dosage used will vary depending on the clinical goals to be achieved, a suitable dosage range is in some embodiments one which provides a mean peak blood or plasma compound 1 concentration (Cₘₐₓ) of at least 50 nM in a blood sample taken from the individual being treated, after administration of the subject dosage form to the individual, wherein the mean time to Cₘₐₓ (Tₘₐₓ) of compound 1 in the blood or plasma is no more than 360 minutes.

In some embodiments, a pharmaceutical dosage form comprising compound 1 is administered in an amount that provides, following administration, a mean blood or plasma Cₘₐₓ of compound 1 of at least 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 13 μM, 13.5 μM, 14 μM, 14.5 μM, 15 μM, 15.5 μM, 16 μM, 16.5 μM, 17 μM, 17.5 μM, 18 μM, 18.5 μM, 19 μM, 19.5 μM, 20 μM, 20.5 μM, 21 μM, 21.5 μM, 22 μM, 22.5 μM, 23 μM, 23.5 μM, 24 μM, 24.5 μM, or at least 25 μM.

In some embodiments, the subject pharmaceutical dosage form is administered in an amount that provides, within 1 day following administration, a mean blood or plasma Cₘₐₓ of compound 1 that is at least 2-fold greater than the Cₘₐₓ achieved with the subject control formulation of compound 1 (e.g., as described herein in Example 4). In some cases, the maximum blood plasma concentration (Cₘₐₓ) achieved after one (1) day is at least 2.1-fold greater than the Cₘₐₓ achieved with a control formulation, e.g., at least 2.2-fold, 2.3-fold, 2.4-fold, 2.5-fold, 3-fold, 3.5-fold, 4-fold, 4.5-fold, 5-fold or 5.5-fold greater than the Cₘₐₓ achieved with a control formulation of compound 1.

In some embodiments, the subject pharmaceutical dosage form is administered in an amount that provides, an area under the curve (AUC) for compound 1 that is at least 2-fold greater than the AUC achieved with the subject control formulation of compound 1 (e.g., as described herein in Example 4). In some cases, the AUC achieved for compound 1 is at least 2.1-fold greater than the AUC achieved for compound 1 with a control formulation, e.g., at least 2.2-fold, 2.3-fold, 2.4-fold, 2.5-fold, 3-fold, 3.5-fold, 4-fold, 4.5-fold, 5-fold or 5.5-fold greater than the AUC achieved with a control formulation of compound 1.

In some embodiments, the subject pharmaceutical dosage form is administered in an amount that provides, an area under the curve (AUC) for a metabolite of compound 1 that is at least 2-fold greater than the AUC achieved with the subject control formulation of compound 1 (e.g., as described herein in Example 4). In some cases, the AUC achieved for the metabolite is at least about 2.1-fold greater than the Cₘₐₓ achieved with a control formulation, e.g., at least 2.2-fold, 2.3-fold, 2.4-fold, 2.5-fold. 3-fold, 3.5-fold, 4-fold, 4.5-fold, 5-fold or 5.5-fold greater than the AUC achieved for the metabolite of compound 1 with a control formulation of compound 1. In some embodiments, the metabolite of compound 1 is 2-(4-(tert-butyl)phenyl)-1H-benzo[d]imidazol-5-ol (compound 2).

In some embodiments, the subject pharmaceutical dosage form administered comprising an amorphous solid dispersion of compound 1 exhibits greater (e.g., 2-fold greater or more) mean peak concentration (Cₘₐₓ) as compared to a dosage form administered as compound 1 reconstituted in a vehicle comprising methylcellulose (0.5% w/w), sodium dodecyl sulfate (0.10% w/w), simethicone emulsion (0.10% w/w) and water (q.s. to 100% w/w). In some cases, the subject pharmaceutical dosage form administered as an amorphous solid dispersion of compound 1 exhibits at least a 2.0-fold, a 2.5-fold, a 3-fold, or at least a 3.5-fold greater mean peak concentration (Cₘₐₓ) as compared to a dosage form administered as compound 1 reconstituted in a vehicle comprising methylcellulose (0.5% w/w), sodium dodecyl sulfate (0.10% w/w), simethicone emulsion (0.10% w/w) and water (q.s. to 100% w/w).

In some embodiments, the subject pharmaceutical dosage form administered comprising an amorphous solid dispersion of compound 1 exhibits greater (e.g., 2-fold greater or more) AUC for compound 1 as compared to a dosage form administered as compound 1 reconstituted in a vehicle comprising methylcellulose (0.5% w/w), sodium dodecyl sulfate (0.10% w/w), simethicone emulsion (0.10% w/w) and water (q.s. to 100% w/w). In certain cases, the pharmaceutical dosage form administered comprising an amorphous solid dispersion of compound 1 exhibits at least a 2.0-fold, a 2.5-fold, a 3-fold, or at least a 3.5-fold greater AUC for compound 1 as compared to a dosage form administered as compound 1 reconstituted in a vehicle comprising methylcellulose (0.5% w/w), sodium dodecyl sulfate (0.10% w/w), simethicone emulsion (0.10% w/w) and water (q.s. to 100% w/w).

In some embodiments, the subject pharmaceutical dosage form administered comprising an amorphous solid dispersion of compound 1 exhibits greater (e.g., 2-fold greater or more) AUC for a metabolite of compound 1 as compared to a dosage form administered as compound 1 reconstituted in a vehicle comprising methylcellulose (0.5% w/w), sodium dodecyl sulfate (0.10% w/w), simethicone emulsion (0.10% w/w) and water (q.s. to 100% w/w). In some cases, the pharmaceutical dosage form administered comprising an amorphous solid dispersion of compound 1 exhibits at least a 2.0-fold, a 2.5-fold, a 3-fold, or at least a 3.5-fold greater AUC for an a metabolite of compound 1 as compared to a dosage form administered as compound 1 reconstituted in a vehicle comprising methylcellulose (0.5% w/w), sodium dodecyl sulfate (0.10% w/w), simethicone emulsion (0.10% w/w) and water (q.s. to 100% w/w). In some embodiments, the metabolite of compound 1 is 2-(4-(tert-butyl)phenyl)-1H-benzo[d]imidazol-5-ol (compound 2).

Depending on the subject and condition being treated and on the administration route, the pharmaceutical dosage form may be administered with compound 1 dosages of, for example, at least 0.5 mg/kg body weight per day. In some embodiments, the compound 1 dose is at least 1 mg/kg body weight per day. In certain embodiments, the dose is at least 2 mg/kg, at least 3 mg/kg, at least 4 mg/kg, at least 5 mg/kg, at least 6 mg/kg, at least 7 mg/kg, at least 8 mg/kg, at least 9 mg/kg, or at least 10 mg/kg body weight per day.

In some embodiments, the pharmaceutical dosage form may be administered with compound 1 dosages of at least 10 mg/kg body weight per day. In certain embodiments, the dose is at least 15 mg/kg, at least 20 mg/kg, at least 25 mg/kg, 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, at least 100 mg/kg, at least 150 mg/kg, at least 175 mg/kg, or at least 200 mg/kg body weight per day. In certain embodiments, the dose is 250 mg/kg, 300 mg/kg, 350 mg/kg, 400 mg/kg, 450 mg/kg, 500 mg/kg, 600 mg/kg, 650 mg/kg, 700 mg/kg, 750 mg/kg, 800 mg/kg, 850 mg/kg, 900 mg/kg, 950 mg/kg, or 1000 mg/kg body weight per day. In certain embodiments, the oral dose is 0.5 mg/kg to 100 mg/kg body weight per day. In certain embodiments, the oral dose is 2 mg/kg to 100 mg/kg body weight per day. In certain embodiments, the oral dose is 25 mg/kg to 1000 mg/kg body weight per day.

In some embodiments, pharmaceutical dosage form may be administered with compound 1 dosages of 25 mg/kg body weight per day. In certain embodiments, the dose is at least 25 mg/kg body weight per day. In certain embodiments, the dose is at least 50 mg/kg, at least 100 mg/kg, at least 150 mg/kg, at least 175 mg/kg, or at least 200 mg/kg body weight per day. In certain embodiments, the dose is 250 mg/kg, 500 mg/kg, 750 mg/kg, or 1000 mg/kg body weight per day. In certain embodiments, the oral dose is 25 mg/kg to 1,000 mg/kg body weight per day.

The dosage range is broad, since in general the efficacy of a therapeutic effect of compound 1 for different mammals varies widely with doses typically being 20, 30 or even 40 times smaller (per unit body weight) in man than in the rat. Similarly, the mode of administration can have a large effect on dosage. Thus, for example, oral dosages may be about ten times the injection dose. Higher doses may be used for localized routes of delivery.

Depending on the subject and condition being treated and on the administration route, the pharmaceutical dosage form may be administered with compound 1 dosages of, for example, 50 to 10,000 mg/dose, e.g., from 50 mg/dose to 100 mg/dose, from 100 mg/dose to 200 mg/dose, from 200 mg/dose to 250 mg/dose, from 300 mg/dose to 350 mg/dose, from 350 mg/dose to 400 mg/dose, from 400 mg/dose to 450 mg/dose, from 450 mg/dose to 500 mg/dose, from 500 mg/dose to 550 mg/dose, from 600 mg/dose to 650 mg/dose, from 650 mg/dose to 700 mg/dose, from 700 mg/dose to 750 mg/dose, from 750 mg/dose to 800 mg/dose, from 800 mg/dose to 850 mg/dose, from 850 mg/dose to 900 mg/dose, from 900 mg/dose to 950 mg/dose, from 950 mg/dose to 1000 mg/dose, from 1,000 mg/dose to 2,500 mg/dose, from 2,500 mg/dose to 5,000 mg/dose, from 5,000 mg/dose to 7500 mg/dose, from 7,500 mg/dose to 10,000 mg/dose. In some embodiments, the pharmaceutical dosage form may be administered in dosages of, for example, 0.1 to 10 g/dose, e.g., from 0.1 g/dose to 0.25 g/dose, from 0.2 g/dose to 0.4 g/dose, from 0.4 g/dose to 0.5 g/dose, from 0.5 g/dose to 1 g/dose, from 1 g/dose to 3 g/dose, from 3 g/dose to 5 g/dose, from 5 g/dose to 6 g/dose, from 6 g/dose to 8 g/dose, from 8 g/dose to 10 g/dose.

In some embodiments, the pharmaceutical dosage form comprises 100-200 mg compound 1, and is administered orally once daily.

In some embodiments, the pharmaceutical dosage form comprises 200-300 mg compound 1, and is administered orally once daily.

In some embodiments, the pharmaceutical dosage form comprises 300-400 mg compound 1, and is administered orally once daily.

In some embodiments, the pharmaceutical dosage form comprises 400-500 mg compound 1, and is administered orally once daily.

### 5.5.1. Therapeutic Indications

Aspects of the present disclosure include methods of treating therapeutic indications of interest using the subject pharmaceutical compositions or dosage forms comprising compound 1 (e.g., as described herein). The term “therapeutic indication” refers to any symptom, condition, disorder, or disease that may be alleviated, stabilized, improved, cured, or otherwise addressed by some form of treatment or other therapeutic intervention with compound 1. In some embodiments, methods of the present disclosure may include treating compound 1-related indications by administering compositions or pharmaceutical dosage forms disclosed herein (e.g., dosage forms comprising compound 1).

Compound 1 has been shown to have use in the treatment and/or prevention of a range of diseases and conditions. Examples include but are not limited to, neurodegenerative diseases. In certain embodiments, the neurodegenerative disease is selected from a motor neuron disease, amyotrophic lateral sclerosis (ALS), Alzheimer's disease, vascular dementia, frontotemporal degeneration (frontotemporal dementia), dementia with Lewy bodies, Parkinson's disease, Huntington's disease, demyelinating disease, and multiple sclerosis (MS).

### 5.6. Definitions

As used herein, the term “solid dispersion” is meant a molecular dispersion of a compound, particularly a drug substance within a carrier. The term solid dispersion in general means a system in solid state comprising at least two components, wherein one component is dispersed substantially evenly throughout the other component(s). For example, solid dispersions may be the dispersion of one or more active ingredients in an inert carrier or matrix at solid state, prepared by the melting, solvent, or melting-solvent methods. While not wishing to be bound by theory, in a solid dispersion, the drug may be present in a molecular state, colloidal state, metastable state, or an amorphous state. Formation of a molecular dispersion may provide a means of reducing the particle size to nearly molecular levels (i.e., there are no particles).

The abbreviation “mg” means milligrams.

The abbreviation “BID” means “twice daily.”

The abbreviation “QD” means “once daily.”

The terms “subject” and “patient” are used interchangeably. A subject can be a mammal such as a non-primate (e.g., cows, pigs, horses, cats, dogs, goats, rabbits, rats, mice, etc.) or a primate (e.g., monkey and human), for example a human. In certain embodiments, the subject is a mammal, e.g., a human, diagnosed with a disease or disorder provided herein. In another embodiment, the subject is a mammal, e.g., a human, at risk of developing a disease or disorder provided herein. In a specific embodiment, the subject is human.

The terms “therapies” and “therapy” are used in their broadest sense understood in the clinical arts.

The term “pharmaceutically acceptable” indicates that the material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables.

The term “carrier” refers to a glidant, diluent, adjuvant, excipient, or vehicle etc. with which the compound is administered, without limitation. Examples of carriers are described herein and also in Remington: The Science and Practice of Pharmacy (Remington: The Science and Practice of Pharmacy, 23rd Edition, ISBN-13: 978-0128200070).

The term “diluent” refers to chemical compounds that are used to dilute the compound of interest prior to delivery. Diluents can also serve to stabilize compounds. Non-limiting examples of diluents include starch, saccharides, disaccharides, sucrose, lactose, polysaccharides, cellulose, cellulose ethers, hydroxypropyl cellulose, sugar alcohols, xylitol, sorbitol, maltitol, microcrystalline cellulose, calcium or sodium carbonate, lactose, lactose monohydrate, dicalcium phosphate, cellulose, compressible sugars, dibasic calcium phosphate dehydrate, mannitol, and tribasic calcium phosphate.

The term “binder” when used herein relates to any pharmaceutically acceptable film which can be used to bind together the active and inert components of the carrier together to maintain cohesive and discrete portions. Non-limiting examples of binders include hydroxypropyl cellulose, hydroxypropylmethylcellulose, povidone, copovidone, and ethyl cellulose.

The term “disintegrant” refers to a substance which, upon addition to a solid preparation, facilitates its break-up or disintegration after administration and permits the release of an active ingredient as efficiently as possible to allow for its rapid dissolution.

Non-limiting examples of disintegrants include maize starch, sodium starch glycolate, croscarmellose sodium, modified corn starch, sodium carboxymethyl starch, crospovidone, pregelatinized starch, and alginic acid.

The term “lubricant” refers to an excipient which is added to a powder blend to prevent the compacted powder mass from sticking to the equipment during the tableting or encapsulation process. It aids the ejection of the tablet form the dies, and can improve powder flow. Non-limiting examples of lubricants include magnesium stearate, stearic acid, silica, fats, calcium stearate, polyethylene glycol, sodium stearyl fumarate, or talc; and solubilizers such as fatty acids including lauric acid, oleic acid, and C8/C10 fatty acid.

The term “film coating” refers to a thin, uniform, film on the surface of a substrate (e.g., tablet). Film coatings are particularly useful for protecting the active ingredient from photolytic degradation. Non-limiting examples of film coatings include polyvinylalcohol based, hydroxyethyl cellulose, hydroxypropylmethylcellulose, sodium carboxymethylcellulose, polyethylene glycol 4000 and cellulose acetate phthalate film coatings.

The term “glidant” as used herein is intended to mean agents used in tablet and capsule formulations to improve flow-properties during tablet compression and to produce an anti-caking effect. Non-limiting examples of glidants include colloidal silicon dioxide, talc, fumed silica, starch, starch derivatives, and bentonite.

The term “effective amount” or “therapeutically effective amount” refers to an amount that is sufficient to effect treatment, as defined herein, when administered to a mammal in need of such treatment. The therapeutically effective amount will vary depending upon the patient being treated, the weight and age of the patient, the severity of the disease condition, the manner of administration and the like, which can readily be determined by one of ordinary skill in the art.

The term “unit dosage forms” or “pharmaceutical dosage forms” refers to physically discrete units suitable as unitary dosages for human patients and other mammals, each unit containing a predetermined quantity of active material calculated to produce the desired therapeutic effect, in association with a suitable pharmaceutical excipient (e.g., a sachet or a tablet).

The term “treatment” or “treating,” to the extent it relates to a disease or condition includes preventing the disease or condition from occurring, inhibiting the disease or condition, eliminating the disease or condition, and/or relieving one or more symptoms of the disease or condition.

The term “% w/w” as used herein refers to the weight of a component based on the total weight of a composition comprising the component. For example, if component A is present in an amount of 50% w/w in a 100 mg composition, component A is present in an amount of 50 mg.

Unless specifically stated otherwise, where a compound may assume alternative tautomeric, regioisomeric and/or stereoisomeric forms, all alternative isomers, are intended to be encompassed within the scope of the claimed subject matter. For example, when a compound is described as a particular optical isomer D- or L-, it is intended that both optical isomers be encompassed herein. For example, where a compound is described as having one of two tautomeric forms, it is intended that both tautomers be encompassed herein. Thus, the compounds provided herein may be enantiomerically pure, or be stereoisomeric or diastereomeric mixtures. The compounds provided herein may contain chiral centers. Such chiral centers may be of either the (R) or (S) configurations, or may be a mixture thereof. The chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

The present disclosure also encompasses all suitable isotopic variants of the compounds according to the present disclosure, whether radioactive or not. An isotopic variant of a compound according to the present disclosure is understood to mean a compound in which at least one atom within the compound according to the present disclosure has been exchanged for another atom of the same atomic number, but with a different atomic mass than the atomic mass which usually or predominantly occurs in nature. Examples of isotopes which can be incorporated into a compound according to the present disclosure are those of hydrogen, carbon, nitrogen, oxygen, fluorine, chlorine, bromine and iodine, such as ²H (deuterium), ³H (tritium), ¹³C, ¹⁴C, ¹⁵N, ¹⁷O, ¹⁸O, ¹⁸F, ³⁶Cl, ⁸²Br, ¹²³I, ¹²⁴I, ¹²⁵I, ¹²⁹I and ¹³¹I. Particular isotopic variants of a compound according to the present disclosure, especially those in which one or more radioactive isotopes have been incorporated, may be beneficial, for example, for the examination of the mechanism of action or of the active compound distribution in the body. Compounds labelled with ³H, ¹⁴C and/or ¹⁸F isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required. In some embodiments, hydrogen atoms of the compounds described herein may be replaced with deuterium atoms. In certain embodiments, “deuterated” as applied to a chemical group and unless otherwise indicated, refers to a chemical group that is isotopically enriched with deuterium in an amount substantially greater than its natural abundance. Isotopic variants of the compounds according to the present disclosure can be prepared by various, including, for example, the methods described below and in the working examples, by using corresponding isotopic modifications of the particular reagents and/or starting compounds therein.

Thus, any of the embodiments described herein are meant to include a salt, a single stereoisomer, a mixture of stereoisomers and/or an isotopic form of the compounds.

Unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 10%, 9%, 8%, 7%, 6%, 5%, 4%, 3%, 2%, 1%, 0.5%, 0.4%, 0.3%, 0.25%, 0.2%, 0.1% or 0.05% of a given value or range. Unless otherwise specified, the term “about” means within plus or minus 10% of a the explicitly recited value, rounded either up or down to the nearest integer.

### 6. EXAMPLES

The Examples in this section are offered by way of illustration, and not by way of limitation. The examples can represent only some embodiments, and it should be understood that the following examples are illustrative and not limiting. All excipients, unless otherwise specified, are as previously defined. The excipients and starting materials are readily available to one of ordinary skill in the art. The specific steps for each of the routes described may be combined in different ways, or in conjunction with steps from different processes, to prepare the formulations described herein.

### 6.1. Example 1: Preparation of a Spray Dried Dispersion (SDD) Formulation of 2-(4-tert-butylphenyl)-1H-benzimidazole (Compound 1)

A spray-dried dispersion (SDD) of 2-(4-tert-butylphenyl)-1H-benzimidazole (compound 1) having the composition set out in Table 1 was prepared by spray drying a feedstock formulation set out in Table 2.

TABLE 1
Spray Dried Dispersion (SDD) Formulation of compound 1

| | | Amount |
|---|---|---|
| Ingredient | % w/w | (g) |
| Compound 1 | 30 | 45.0 |
| polyvinyl caprolactam-polyvinyl acetate-polyethylene | 40 | 60.0 |
| glycol graft copolymer (Soluplus) | | |
| amorphous silicon dioxide (Syloid ® 244 FP) | 30 | 45.0 |
| Total | 100 | 150 |

TABLE 2
Spray Drying Feedstock Formulation

| | | Amount |
|---|---|---|
| Ingredient | % w/w | (g) |
| Compound 1 | 2.3 | 45.0 |
| polyvinyl caprolactam-polyvinyl acetate-polyethylene | 3.1 | 60.0 |
| glycol graft copolymer (Soluplus) | | |
| amorphous silicon dioxide (Syloid ® 244 FP) | 2.3 | 45.0 |
| 2-propanol | 92.3 | 1797.1 |
| Total | 100 | 1947.1 |

### Manufacturing Procedure for Spray Drying Feedstock Formulation

Compound 1 (45.0 g) was slowly added to 2-propanol (1791.1 g) with stirring, placed under a homogenizer (Silverson SL2 homogenizer) and stirred for 5 minutes or more until Compound 1 was fully dissolved. The reaction mixture was then removed from the homogenizer and polyvinyl caprolactam-polyvinyl acetate-polyethylene glycol graft co-polymer (Soluplus) (60.0 g) was slowly added with stirring, placed back under the homogenizer and stirred for 10 minutes or more until the Soluplus was fully dissolved. The reaction mixture was then removed from the homogenizer and amorphous silicon dioxide (Syloid® 244 FP) was slowly added with stirring, placed back under the homogenizer and stirred for an additional 15 minutes or more until the amorphous silicon dioxide was fully dispersed. The resulting suspension is referred to herein as the “Feedstock Formulation.”

### Manufacturing Procedure for Spray Dried Dispersion (SDD) Formulation of Compound 1

The spray dryer unit (ProCepT 4M8 Spray Dryer) was set up with a compressed air supply. Once the outlet temperature stabilized, the feed pump was initiated and 2-propanol (blank solution) was sprayed through the nozzle as a fine spray into the collection chamber. The spray dryer parameters were adjusted to achieve a feed rate within the range set out in Table 3 below.

The feedstock formulation was stirred under a homogenizer at a speed appropriate to maintain a homogenous dispersion without generating bubbles. The feedstock formulation was then sprayed through the nozzle as a fine spray into the collection chamber of the spray dryer unit (ProCepT 4M8 Spray Dryer, using parameters as set up with the blank solution and outlined in Table 3) where the solvent was evaporated quickly to generate particles containing compound 1 polyvinyl caprolactam-polyvinyl acetate-polyethylene glycol graft co-polymer (Soluplus) and silicon dioxide (Syloid® 244 FP) (SDD formulation of compound 1). Once all the feedstock formulation had been sprayed and collected, the feedstock formulation was replaced with 2-propanol (blank solution) and sprayed through the nozzle of the spray dryer for 5 minutes or more to allow collection of any remaining “feedstock formulation” within the air stream.

TABLE 3
Target Process Parameters for Spray Dryer

| Process Parameter | Target | |
|---|---|---|
| Inlet air flow | 0.45 | m³/min |
| Inlet air temperature | 88° | C. |
| Suspension feed rate | 4-7 | g/min |
| Peristaltic tubing | 50513 Tygon MHLL ID 2.79 | |
| | mm (2-stop, purple - white) | |
| Nozzle diameter | 0.6 | mm |
| Nozzle cap | with cap | |
| Nozzle pressure | 2 | bar |
| Cyclone gas pressure | 0.3 | bar |

### 6.2. Example 2: Oral Suspension of a Spray Dried Dispersion (SDD) Formulation of 2-(4-tert-butylphenyl)-1H-benzimidazole (Compound 1)

The spray-dried dispersion (SDD) of 2-(4-tert-butylphenyl)-1H-benzimidazole (compound 1) having the composition set out in Table 1 (e.g., from 60-1000 mg) was reconstituted as an oral suspension in 100 g of vehicle composed of PEG 300 (10 g), glycerol monocaprylocaprate (Capmul MCM, 0.40 mg) in sterile water for irrigation (q.s. to 100 g).

### 6.3. Example 3: Preparation of a Hot-Melt Extrusion (HME) Formulation of 2-(4-tert-butylphenyl)-1H-benzimidazole (Compound 1)

A hot-melt extrusion (HME) formulation of 2-(4-tert-butylphenyl)-1H-benzimidazole (compound 1) having the composition set out in Table 3 was prepared as set out below.

TABLE 4
Hot-Melt Extrusion (HME) Formulation of compound 1

| | | Amount | Amount |
|---|---|---|---|
| | | (mg) for | (mg) for |
| | | 60 mg | 1000 mg |
| Ingredient | % w/w | dose | dose |
| Compound 1 | 25 | 60 | 1000 |
| Povidone (Kollidon 17PF) | 25 | 60 | 1000 |
| Copovidone (Kollidon VA64) | 25 | 60 | 1000 |
| polyvinyl caprolactam-polyvinyl acetate- | 25 | 60 | 1000 |
| polyethylene glycol graft co-polymer | | | |
| (Soluplus) | | | |
| Total | 100 | 240 | 4000 |

### Manufacturing Procedure

The required quantities of compound 1, polyvinyl caprolactam-polyvinyl acetate-polyethylene glycol graft co-polymer (Soluplus), povidone (Kollidon 17PF) and copovidone (Kollidon VA64) according to table 4 were weighed, passed through an 850 μm sieve and transferred to a 2 L blender shell. The resultant blender shell was secured in a blender (Pharmatech blender) and blended for 20 minutes then added to a double polyethylene (PE) bag (“compound 1 blend”) and transferred to the HME containment system. A chiller unit was connected to the HME and once the chiller temperature reached 15° C. the extrusion process was commenced using the parameters outlined in Table 5. The compound 1 blend was added to the feeder to fill approximately ¾ of the feeder, and the extrudate was collected and discarded for approximately the first 5 minutes of the extrusion process. The feeder was refilled to maintain approximately 50% volume in the feeder throughout the process, and extrusion was continued until all the compound 1 blend was extruded and collected (“compound 1 HME extrudate”).

TABLE 5
HME parameters

| | | |
|---|---|---|
| Feed rate | 0.110-0.175 | kg/hour |

| Temperature | Zone 2 | Zone 3 | Zone 4 | Zone 5 | Zone 6 | Zone 7 | Zone 8 | Die |
|---|---|---|---|---|---|---|---|---|
| (all zones) | 50-70° C. | 80-170° C. | 200-220° C. | 220-240° C. | 220-240° C. | 220-240° C. | 220-240° C. | 170-195° C. |

| | | |
|---|---|---|
| Screw speed | 30 | rpm |
| Die Dimension | 1.0 | mm |
| Chiller temperature | 15° | C. |

The collected compound 1 HME extrudate was added to a U5 Quadro mill (set up with a screen size of 457 (mm) and an impeller speed of 5000 RPM), until all extrudate had passed the 457 mm screen to obtain milled granules of compound 1. The milled granules of compound 1 were then sieved using a 300 micron sieve and transferred into a blender shell (Pharmatech 2 L blender shell). The resultant blender shell was secured in a blender (Pharmatech blender), blended for 5 minutes, and collected.

### 6.4. Example 4: Oral Suspension of a Hot-Melt Extrusion (HME) Formulation of 2-(4-tert-butylphenyl)-1H-benzimidazole (Compound 1)

The hot-melt extrusion (HME) of 2-(4-tert-butylphenyl)-1H-benzimidazole (compound 1) having the composition set out in Table 4 (e.g., from 60-1000 mg) was reconstituted as an oral suspension in 100 mL of vehicle Ora-Blend SF® (purified water, sucrose, glycerin, sorbitol, flavoring, microcrystalline cellulose, sodium carboxymethylcellulose, xanthan gum, carrageenan, citric acid, sodium phosphate, simethicone, potassium sorbate and methylparaben), a commercially available oral suspending vehicle manufactured by Perrigo Pharmaceuticals.

### 6.5. Example 5: Preparation of a Spray Dried Dispersion (SDD) Blend Formulation of 2-(4-tert-butylphenyl)-1H-benzimidazole (Compound 1)

### General Manufacturing Procedure for SDD Blend Formulations

The required amount of compound 1 SDD formulation (e.g., as prepared in accordance with Example 1) was weighed, sieved through a 250 μm sieve and transferred to a suitable blending shell. The required amount of diluent was then weighed, sieved through a 250 μm sieve and transferred to the suitable blending shell with the compound 1 SDD formulation (“the blending shell”). The resultant mixture was blended for 3 minutes at 25 rpm (Pharmatech blender). The disintegrant, sweetener and lubricant were then weighed, sieved through a 250 μm sieve and transferred to the blending shell. The resultant mixture was blended at 25 rpm (Pharmatech blender) for approximately 60 minutes. The SDD blends of compound 1 were then evaluated for bulk density, tapped density, Carr's Index and Hausner ratio (see Tables 8-10 below) before filling into sachets. Sachets of each blend (10 sachets of each) were then submitted for content uniformity testing (see Table 11 below).

The compound 1 SDD blend formulation compositions are summarized in Tables 6-7.

TABLE 6
Compound 1 SDD blend formulation compositions 1-3

| | Batch No. | | | | | | |
|---|---|---|---|---|---|---|---|
| | | | 1 + 2% | | | | |
| | | | colloidal | | | | |
| | 1 | | silica | 2 | | 3 | |
| Excipients | mg/sachet | % w/w | mg/sachet | mg/sachet | % w/w | mg/sachet | % w/w |
| Compound 1 SDD | 400.0 | 40.0 | 400.0 | 400.0 | 40.0 | 400.0 | 40.0 |
| Microcrystalline cellulose | 588.5 | 58.85 | 588.5 | | | | |
| (particle size = 100 μm) | | | | | | | |
| Partially pregelatinized | | | | 588.5 | 58.85 | | |
| starch (Starch 1500) | | | | | | | |
| Mannitol (100 SD) | | | | | | 588.5 | 58.85 |
| Sodium stearyl fumarate | 10.0 | 1.00 | | | | | |
| Sucralose | 1.5 | 0.15 | 1.5 | 1.5 | 0.15 | 1.5 | 0.15 |
| Colloidal silica (specific | | | 20.0 | | | | |
| surface area = 200 m²/g) | | | | | | | |
| Total | 1000 | 100 | 1020 | 1000 | 100 | 1000 | 100 |

TABLE 7
Compound 1 SDD blend formulation compositions 4-5

| | Batch No. | | | |
|---|---|---|---|---|
| | 4 | | 5 | |
| | mg/ | | mg/ | |
| Excipients | sachet | % w/w | sachet | % w/w |
| Compound 1 SDD | 400.0 | 40.0 | 400.0 | 40.0 |
| Microcrystalline | | | 502.50 | 50.25 |
| cellulose (particle size = | | | | |
| 50 μm) | | | | |
| Silicified | 567.50 | 56.75 | | |
| microcrystalline cellulose | | | | |
| (SMCC HD 90) | | | | |
| Croscarmellose sodium | | | 50.0 | 5.00 |
| (Ac-Di-Sol) | | | | |
| Sodium stearyl fumarate | 10.0 | 1.0 | 10.0 | 1.0 |
| Sucralose | 22.50 | 2.25 | 22.50 | 2.25 |
| Colloidal silica (specific | | | 15 | 1.50 |
| surface area = 200 m²/g) | | | | |
| Total | 1000 | 100 | 1000 | 100 |

The results for bulk density, tapped density, Carr's Index and Hausner ratio for the compound 1 SDD blend formulation compositions are summarized in Tables 8 and 9.

TABLE 8
Flow properties for Compound 1 SDD
blend formulation compositions 1-3

| Flow Properties | 1 | 1 + 2% colloidal silica | 2 | 3 |
|---|---|---|---|---|
| Bulk Density (mg/mL) | 0.36 | 0.32 | 0.40 | 0.34 |
| Tapped Density (mg/mL) | 0.57 | 0.52 | 0.63 | 0.56 |
| Carr Index | 36.36 | 38.24 | 36.67 | 40.00 |
| Hausner Ratio | 1.57 | 1.62 | 1.58 | 1.67 |

TABLE 9
Flow properties for Compound 1 SDD blend formulation
compositions 4-5 and Compound 1 SDD

| Flow Properties | Compound 1 SDD | 4 | 5 |
|---|---|---|---|
| Bulk Density (mg/mL) | 0.36 | 0.36 | 0.33 |
| Tapped Density (mg/mL) | 0.57 | 0.53 | 0.52 |
| Carr Index % | 36.36 | 32 | 37 |
| Hausner Ratio | 1.57 | 1.47 | 1.58 |

Table 10 shows the relationship between Carr Index (compressibility index) and powder flow properties.

TABLE 10
Relationship between Carr Inex and powder flow properties

| Carr Index (%) | Flow Character | Hausner Ratio |
|---|---|---|
| ≤10 | Excellent | 1.00-1.11 |
| 11-15 | Good | 1.12-1.18 |
| 16-20 | Fair | 1.19-1.25 |
| 21-25 | Passable | 1.26-1.34 |
| 26-31 | Poor | 1.35-1.45 |
| 32-37 | Very Poor | 1.46-1.59 |
| ≥38 | Very, Very Poor | ≥1.6 |

Table 11 shows the results of content uniformity testing for Compound 1 SDD blend formulations.

TABLE 11
Content Uniformity Results for Compound
1 SDD blend formulation compositions 1-5

| Blend | Mean % Content Uniformity (CU) |
|---|---|
| 1 | 92.6 |
| 2 | 91.0 |
| 3 | 90.9 |
| 4 | 91.2 |
| 5 | 94.3 |

### 6.6. Example 6: Control Formulation—Compound 1 Methylcellulose (MC) Powder Suspension Formulation (“MC Formulation”)

A methylcellulose (MC) powder suspension formulation of 2-(4-tert-butylphenyl)-1H-benzimidazole (compound 1) was prepared by reconstituting compound 1 as a suspension in a methylcellulose vehicle formulation of Table 12 (“the vehicle formulation”).

TABLE 12
Vehicle formulation for preparation of MC
powder suspension formulation of compound 1

| Component | % w/w | Amount (g) |
|---|---|---|
| methylcellulose (Methocel | 0.5 | 10.0 |
| A4C Premium, supplied by | | |
| Dupont, yields viscosity of | | |
| 400 cP) | | |
| sodium dodecyl sulfate | 0.10 | 2.0 |
| (Kolliphor SLS Fine, BASF | | |
| 151-21-3) | | |
| 30% simethicone emulsion | 0.10 | 2.0 |
| (supplied by Dow-Corning, | | |
| Q7-2587 oil in water (O/W) | | |
| emulsion with 30.4% | | |
| polydimethylsiloxane | | |
| (PDMS), 1.2-2.1% SiO₂ | | |
| particles) | | |
| water | q.s. to 100 | q.s. to 2000.0 g |
| Total | 100 | 2000 |

The Vehicle formulation was prepared by healing the water (1986 g) to 80° C. (+5° C.) then adding the methylcellulose (10 g) and stirring for 30 minutes or more until the methyl cellulose is fully dispersed. Sodium dodecyl sulfate (2 g) and 30% simethicone emulsion (2 g) were then added, and the mixture was stirred until a translucent, white/off-white, slightly viscous suspension, free form particulate was formed. The pH of the resulting vehicle formulation was 5.3 (target pH was 6.0+/−3.0).

### General Procedure for Reconstitution of Compound 1 in the Vehicle Formulation of Table 12

Required amount of 2-(4-tert-butylphenyl)-1H-benzimidazole (compound 1) (e.g., from 60 mg-1000 mg) was weighed into a vial. The vehicle formulation (100 mL) was added to the vial containing compound 1 to obtain the compound 1 methylcellulose (MC) powder suspension formulation.

### 6.7. Example 7: Pharmacokinetic Study of SDD and HME Formulations of (Compound 1) Vs a Control Formulation

### Single Ascending Dose (SAD) Phase 1 Trial—Double-Blind Randomized Study of Subjects Receiving Single Ascending Doses of Compound 1 or Placebo

A double-blind, randomized, placebo-controlled clinical study was conducted to characterize and compare the pharmacokinetic (PK) profile of Compound 1 and its metabolite Compound 2 following single ascending doses of Compound 1, presented in 3 different formulations (e.g., SDD formulation, HME formulation, or control formulation (MC formulation)), or placebo, in healthy subjects. The three formulations along with reference to the relevant Example number according to which they were prepared are summarized in Table 13 below.

TABLE 13
Formulations of Compound 1 used in SAD phase 1 trail

| | Type of | Prepared in accordance |
|---|---|---|
| Formulation | Formulation | with Example number |
| SDD Formulation | Oral suspension | 1 and 2 |
| HME Formulation | Oral suspension | 3 and 4 |
| MC Formulation (control) | Oral suspension | 6 |

The oral suspension formulations outlined in Table 13 will be referred to throughout Examples 7 and 8 as “SDD formulation,” “HME formulation” and “MC formulation.”

Subjects: This randomized, double-blind, placebo-controlled phase 1 single ascending dose [SAD]trial was conducted in healthy male subjects aged 18 to 55 years with body mass index (BMI) 18.0 to 32.0 kg/m² as measured at screening. Subjects all weighed at least 55 kg at screening. Key criteria for exclusion were subjects with evidence of current SARS-CoV-2 infection, clinical manifestation of significant cardiovascular, renal, hepatic, dermatological, chronic respiratory or gastrointestinal disease, or aspartate aminotransferase (AST) or alanine aminotransferase (ALT)>1.5× the upper limit of normal (ULN). Subjects were recruited at a single site in the United Kingdom. Each subject was provided written informed consent.

Trial Design: The trial was performed in multiple cohorts with a minimum of 7 subjects in each. In Cohorts 1-3 of, subjects received a single oral dose of the MC formulation, or the SDD formulation, or the HME formulation or placebo in the fasted state. Subjects were allocated to study treatment in a ratio of 6 compound 1 formulation to 2 placebo per cohort. Regimens A-E for Cohorts 1-3 (Period 1 and 2) are summarized in Table 14.

TABLE 14
Description of Regimens A-E

| | Period | | | Dose of |
|---|---|---|---|---|
| Cohort | (Dosing Day) | Regimen | Formulation | Compound 1 |
| 1 | N/A | A | MC Formulation | 60 mg |
| | | | or placebo | |
| 2 | N/A | B | MC Formulation | 180 mg |
| | | | or placebo | |
| 3 | 1 (Day 1) | C | MC Formulation | 540 mg |
| | | | or placebo | |
| | 2 (Day 1) | D | SDD Formulation | 180 mg |
| | | | or placebo | |
| | 2 (Day 3) | E | HME Formulation | 180 mg |
| | | | or placebo | |

For Cohorts 1-3 Period 1, a screening period was 4 weeks. After confirming eligibility, subjects within each cohort were randomly assigned to receive either the active (compound 1 formulation) or placebo treatment. Note, this is the first time compound 1 has been dosed in humans, and therefore a sentinel dosing design was followed. Each cohort was split into a sentinel group and a main group. The sentinel group consisted of the first two subjects of each cohort. They were dosed prior to the remaining subjects, the main group. Only after a positive review of the safety data of the sentinel group up to 24 h post-dose were the main group subjects in the cohort dosed. The randomization schedule was constructed such that one of the subjects dosed on the first day received the compound 1 formulation and one received the placebo. Per protocol, all treatments were to be taken once daily following an overnight fast (10 hours fasted). For Cohorts 1-3 Period 1, subjects were admitted in the morning on the day before dosing (Day −1) and remained onsite until their discharge, 48 hours post-dose (Day 3). For Cohorts 3 Period 2, subjects were admitted in the morning on the day before dosing (Day −1) and remained onsite until their discharge, 48 hours post-dose (Day 5). Per protocol, subjects were dosed on the morning of Day 1 either in the fasted state, following an overnight fast (≥10 hours) or fed state, following a high-fat breakfast given 30 minutes before dosing, depending on their assigned regimen.

Post-dosing of Cohorts 1-3 Period 1, and prior to dosing subjects in Cohort 3 Period 2, an interim period was observed, and a safety review was performed. The review concluded that it was safe to dose subjects with the SDD and HME formulations at similar dosage as the MC formulation. As a result, sentinels were not required for Cohort 3, Period 2.

In cohorts 4-6, each subject received a single oral dose of the SDD formulation or placebo in the fed or fasted state. Subjects were randomly allocated to study treatment versus placebo per cohort in a 5:1 ratio. Regimens F-H for Cohorts 4-6 are summarized in Table 15.

TABLE 15
Description of Regimens F-H

| | | | Dose of | |
|---|---|---|---|---|
| Cohort | Regimen | IMP | Compound 1 | State |
| 4 | F | SDD Formulation or | 90 mg | Fed |
| | | placebo | | |
| 5 | G | SDD Formulation or | 90 mg | Fasted |
| | | placebo | | |
| 6 | H | SDD Formulation or | 270 mg | Fasted |
| | | placebo | | |

Per protocol, subjects were dosed on the morning of Day 1 either in the fasted state following an overnight fast (≥10 hours), or fed state following a high-fat breakfast given 30 minutes before dosing.

Safety was continually assessed throughout the trial by monitoring adverse events and concomitant medication use, electrocardiograms (ECGs), vital signs, laboratory safety assessments and physical examinations. Blood samples for pharmacokinetic assessments were collected from each subject from Day −1, prior (≤1 hr) to each dose, and at intervals throughout the study until 48 hours post-dose as applicable. In Cohorts 1-5, no dose was used that was expected to exceed the pre-trial designated exposure cap of Cₘₐₓ 305 ng/mL and AUC₍₀₋₂₄₎ of 2750 ng*h/mL, in any individual subject. After the protocol amendment, this exposure cap was increased to Cₘₐₓ 1121 ng/mL and AUC 5969 ng*h/mL.

Assessment of Pharmacokinetics: Blood samples for plasma PK analysis were collected at regular time intervals. Venous blood samples were collected from the subjects by a trained member of the clinical team. Pre-dose samples were taken ≤1 h before dosing. Timestamp 0 to 1 hour post-dose samples were taken within ±2 minutes of the nominal post-dose sampling time. Timestamp 1.5 to 12 hour post-dose samples were taken within +10 min of the nominal post-dose sampling time. Timestamp 16 to 48 hour post-dose samples will be taken within ±30 minutes of the nominal post-dose sampling time. Samples were collected into appropriate containers and were processed to isolate plasma. PK analysis were carried out on plasma samples using validated bioanalytical methods.

Statistical Analyses: The sample sizes for the study were chosen based on practical considerations and experience from previous studies of a similar design. The numbers of subjects in each cohort (group) were considered to be adequate to assess the main objectives of each study. Pharmacokinetic parameters were determined by non-compartmental techniques using WinNonlin software version 8.0 or higher (Certara USA. Inc., USA). All data were listed and summarized by subject group using descriptive statistics. All statistical analyses were conducted using SAS version 9.4 or higher.

### MC Formulation (Control Formulation) Results

Healthy male subjects were administered a single oral dose of the MC Formulation at one of the following doses in the fasted state: 60 mg, 180 mg or 540 mg of compound 1 (e.g., according to Regimen A-C of Table 14). Plasma concentrations of Compound 1 and metabolite Compound 2 were measured over time, and key pharmacokinetic parameters determined.

Table 16 and Table 17 present the geometric mean of compound 1 and metabolite compound 2 key pharmacokinetic parameters in the subjects following oral administration of compound 1 (MC formulation).

TABLE 16
Geometric Mean (CV %) key pharmacokinetic parameters methylcellulose formulation

| | | Tmax* | Cmax | AUC(0-24) | AUC(0-last) | AUC(0-inf) | T_{1/2} |
|---|---|---|---|---|---|---|---|
| Treatment | Analyte | (h) | (ng/mL) | (ng*h/mL) | (ng*h/mL) | (ng*h/mL) | (h) |
| Cohort 1 Regimen A | Compound 1 | 1.00 | 26.7 | 71.2 | 67.6 | 72.7 | 3.06 |
| 60 mg compound 1, | | (0.5-2.0) | (46.6) | (52.2) | (54.2) | (53.4) | (61.1) |
| MC Formulation | Compound 2 | 2.50 | 65.3 | 383 | 401 | 410 | 4.89 |
| | | (1.0-4.0) | (37.5) | (76.4) | (81.7) | (81.4) | (69.7) |
| Cohort 2 Regimen B | Compound 1 | 1.00 | 53.1 | 163 | 168 | 180 | 7.36 |
| 180 mg compound 1, | | (1.00-4.00) | (55.0) | (46.3) | (51.8) | (53.1) | (75.5) |
| MC Formulation | Compound 2 | 1.50 | 199 | 1210 | 1320 | 1350 | 9.10 |
| | | (1.00-4.00) | (33.5) | (42.2) | (45.9) | (45.1) | (85.2) |
| Cohort 3 Period 1 | Compound 1 | 2.25 | 323 | 1440 | 1540 | 1600 | 10.1 |
| Regimen C 540 mg | | (1.00-10.0) | (70.0) | (74.5) | (71.0) | (67.6) | (81.8) |
| compound 1, MC | Compound 2 | 4.00 | 1110 | 10800 | 12000 | 12700 | 11.3 |
| Formulation | | (2.00-12.00) | (42.5) | (68.2) | (68.9) | (64.9) | (73.0) |

*Median (min-max)

TABLE 17
Geometric Mean (CV %) key pharmacokinetic
parameters methylcellulose formulation

| | | Cmax | MPR | AUC(0-inf) | MPR |
|---|---|---|---|---|---|
| Treatment | Analyte | (ng/mL) | Cmax | (ng · h/mL) | AUC(0-inf) |
| Cohort 1 Regimen A | Compound 1 | 26.7 | — | 72.7 | — |
| 60 mg compound 1, | | (46.6) | | (53.4) | |
| MC Formulation | Compound 2 | 65.3 | 2.44 | 410 | 5.63 |
| | | (37.5) | (53.0) | (81.4) | (38.5) |
| Cohort 2 Regimen B | Compound 1 | 53.1 | — | 180 | — |
| 180 mg Compound 1, | | (55.0) | | (53.1) | |
| MC Formulation | Compound 2 | 199 | 3.74 | 1350 | 7.52 |
| | | (33.5) | (65.1) | (45.1) | (29.4) |
| Cohort 3 Period 1 | Compound 1 | 323 | — | 1600 | — |
| Regimen C 540 mg | | (70.0) | | (67.6) | |
| Compound 1, MC | Compound 2 | 1110 | 3.43 | 12700 | 7.91 |
| Formulation | | (42.5) | (40.3) | (64.9) | (19.1) |

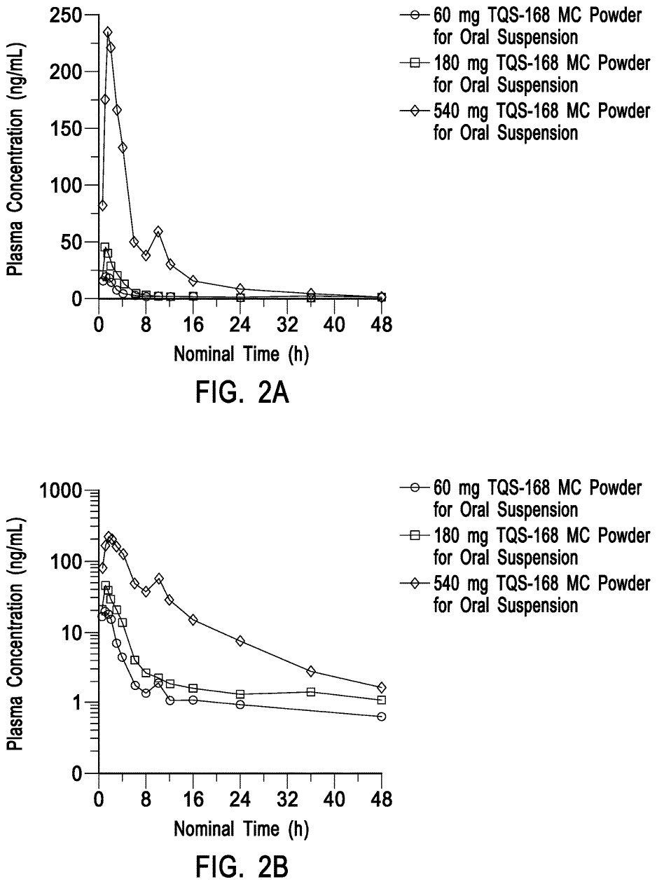

Compound 1 single ascending dose (SAD) PK profile: Cohort 3 Period 1 received 540 mg compound 1 (Regimen C) and provided the highest compound 1 Cₘₐₓ of 323 ng/mL (1.29 μM). Cohort 2 received 180 mg compound 1 (Regimen B) and provided a compound 1 Cₘₐₓof 53.1 ng/mL (0.21 μM). Cohort 1 received 60 mg compound 1 (Regimen A) and provided a compound 1 Cₘₐₓ of 26.7 ng/mL (0.11 μM). Results are plotted in FIGS. 2A-2B. Cohort 3 Period 1 also displayed higher exposure (AUC₀₋₂₄=1440 hr*ng/mL, AUC_{0-inf}=1600 hr*ng/mL) than Cohort 2 (AUC₀₋₂₄=163 hr*ng/m, AUC_{0-inf}=180 hr*ng/mL) and Cohort 1 (AUC₀₋₂₄=71.2 hr*ng/mL, AUC_{0-inf}=72.7 hr*ng/mL).

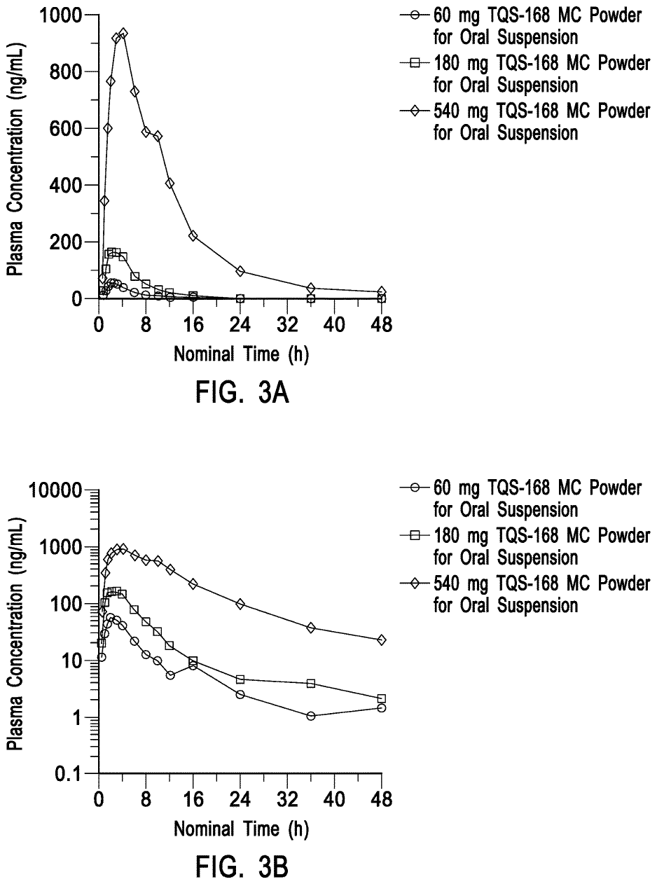

Metabolite Compound 2 PK profile: Cohort 3 Period 1 displayed higher metabolite compound 2 Cₘₐₓ (1110 ng/mL) (4.17 μM) than Cohort 2 (199 ng/mL) (0.75 μM) and Cohort 1 (65.3 ng/mL) (0.25 μM). Results are plotted in FIGS. 3A-3B. Similarly, Cohort 3 also displayed higher metabolite exposure (AUC₀₋₂₄=10800 hr*ng/m, AUC_{inf}=12700 hr*ng/m) than Cohort 2 (AUC₀₋₂₄=1210 hr*ng/mL, AUC_{inf}=1350 hr*ng/mL) and Cohort 1 (AUC₀₋₂₄=383 hr*ng/mL, AUC_{inf}=410 hr*ng/mL).

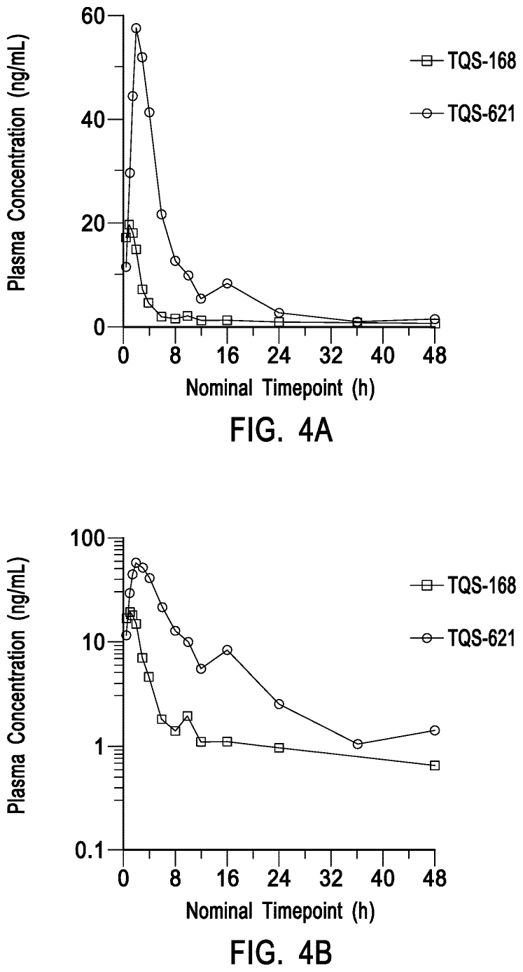

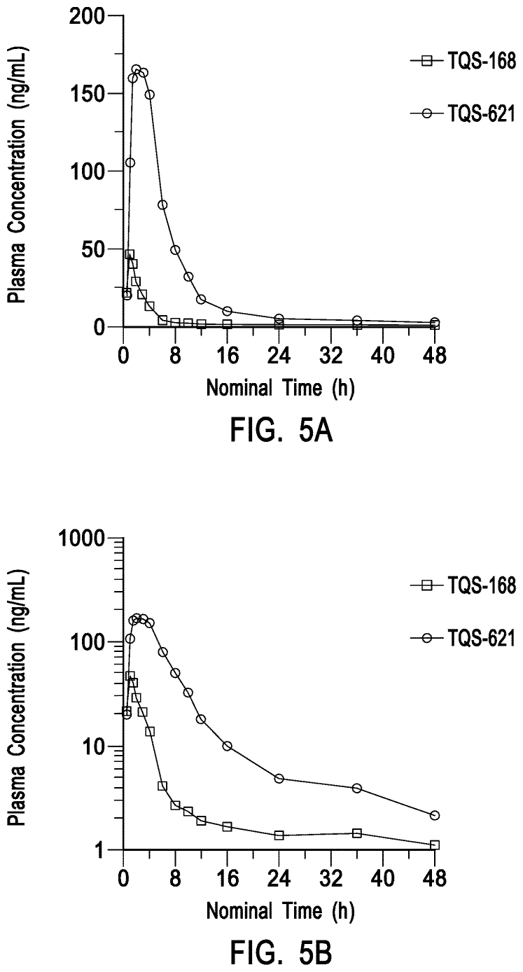

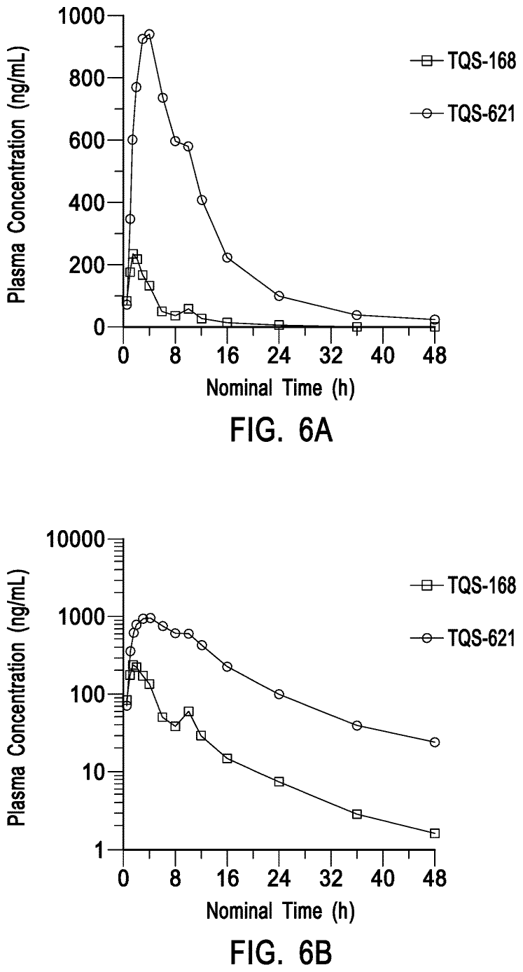

In the study, maximal plasma concentrations for compound 1 (Cₘₐₓ), and compound 1 exposures (AUC₍₀₋₂₄₎ and AUC_{(0-inf)} appeared to increase slightly sub-proportionally to dose following single doses of 60 and 180 mg compound 1. Note, however, that between doses 180 mg and 540 mg of compound 1, Cₘₐₓ, AUC₍₀₋₂₄₎ and AUC_{(0-inf)} for compound 1 increased in a supra-proportional manner, with a 6.1-, 8.8- and 9.5-fold increase, respectively for a 3-fold increase in dose. When observing the entire dose range from 60 mg-540 mg, Cₘₐₓ, AUC₍₀₋₂₄₎ and AUC_{(0-inf)} all increased supra-proportionally by 12.1-fold, 20.2-fold and 21.2-fold respectively. See FIGS. 2A-2B, 4A-6B.

Regarding metabolite compound 2, plasma Cₘₐₓ, AUC₍₀₋₂₄₎ and AUC_{(0-inf)} appeared to increase proportionally to dose following single doses from 60 to 180 mg compound 1. For doses between 180 and 540 mg, Cₘₐₓ, AUC₍₀₋₂₄₎ and AUC_{(0-inf)} increased supra-proportionally with a 5.6-, 8.9- and 9.4-fold increase, respectively for a 3-fold increase in dose. Furthermore, over the entire dose range, a 9-fold increase in dose from 60 mg to 540 mg, plasma Cₘₐₓ, AUC₍₀₋₂₄₎ and AUC_{(0-inf)} increased supra proportionally by 17.0-, 28.2- and 31.0-fold respectively. See FIGS. 3A-6B.

Following a dose of 60 mg, maximum plasma compound 1 concentrations occurred between 0.5 and 2 hours post-dose, with a median Tₘₐₓ of 1 hour post-dose for compound 1. Maximum plasma metabolite compound 2 concentrations occurred between 1-4 hours post dose, with a median Tₘₐₓ of 2.5 hours post-dose.

Following a dose of 180 mg compound 1, the maximum plasma concentrations of compound 1 occurred between 1 and 4 hours post-dose. A median Tₘₐₓ of 1 hour post-dose was observed. Maximum plasma metabolite compound 2 concentrations occurred between 1-4 hours post dose, with a median Tₘₐₓ of 1.5 hours post-dose.

Following administration of 540 mg compound 1, the maximum plasma concentration of compound 1 occurred between 1 and 10 hours post-dose, with a median Tₘₐₓ of 2.25 hours post-dose. Metabolite compound 2 displayed maximum plasma concentrations between 2 and 12 hours post dose, with a median Tₘₐₓ of 4 hours post-dose.

At all administered doses of compound 1, the concentrations of metabolite compound 2 exceeded concentrations of compound 1 following a short lag.

The geometric mean terminal half-life of compound 1 following the compound 1 60 mg dose, 180 mg dose and 540 mg dose was dose-dependent at 3.06, 7.36 and 10.1 hours, respectively. The geometric mean terminal half-life of metabolite compound 2 for compound 1 doses 60 mg, 180 mg and 540 mg was 4.89, 9.10 and 11.3 hours, respectively. It is noted that for all doses of compound 1, plasma concentrations of compound 1 were quantifiable from 0.5 hours post-dose and remained quantifiable up to the final sample time point of 48 hours post-dose. Concentrations of compound 2 were also quantifiable from 0.5 hours post-dose and remained quantifiable up to the final sampling time point of 48 hours post-dose.

### SDD Formulation Results

Healthy male subjects were administered a single oral dose of the SDD formulation at 90 mg or 180 mg or 270 mg of compound 1 in the fed or fasted state (e.g., according to Regimen D of Table 14 and Regimens F-H of Table 15). Plasma concentrations of compound 1 and metabolite compound 2 were measured over time and key pharmacokinetic parameters determined.

Table 18 and Table 19 present compound 1 and metabolite compound 2 key pharmacokinetic parameters in the subjects following oral administration of the SDD formulation.

TABLE 18
Geometric Mean (CV %) key pharmacokinetic parameters SDD formulation

| | | Tmax* | Cmax | AUC(0-24) | AUC(0-last) | AUC(0-inf) | T_{1/2} |
|---|---|---|---|---|---|---|---|
| Treatment | Analyte | (h) | (ng/mL) | (ng · h/mL) | (ng · h/mL) | (ng · h/mL) | (h) |
| Cohort 3 Period 2 | Compound 1 | 1.00 | 218 | 608 | 611 | 621 | 5.64 |
| Regimen D 180 mg | | (0.50-2.00) | (77.2) | (78.5) | (78.8) | (77.4) | (32.4) |
| compound 1, SDD | Compound 2 | 2.00 | 742 | 4860 | 5110 | 5170 | 7.12 |
| Formulation fasted | | (1.50-3.00) | (39.9) | (37.3) | (35.8) | (35.2) | (32.4) |
| state | | | | | | | |
| Cohort 4 Regimen F | Compound 1 | 1.50 | 47.1 | 168 | 166 | 171 | 3.52 |
| 90 mg compound 1, | | (0.50-3.00) | (39.1) | (53.5) | (54.2) | (53.3) | (35.8) |
| SDD Formulation, | Compound 2 | 3.50 | 122 | 848 | 894 | 901 | 5.17 |
| Fed State (high | | (1.50-6.00) | (36.1) | (57.6) | (61.6) | (61.5) | (47.5) |
| fat breakfast) | | | | | | | |
| Cohort 5 Regimen G | Compound 1 | 0.50 | 111 | 192 | 189 | 195 | 4.85 |
| 90 mg compound 1, | | (0.50-4.00) | (92.2) | (58.5) | (60.0) | (58.3) | (45.1) |
| SDD Formulation, | Compound 2 | 1.25 | 214 | 1170 | 1190 | 1200 | 4.79 |
| Fasted State | | (1.00-3.00) | (44.7) | (50.4) | (52.5) | (52.4) | (58.3) |
| Cohort 6 Regimen H | Compound 1 | 1.50 | 237 | 736 | 801 | 836 | 10.4 |
| 270 mg compound 1, | | (0.500-4.00) | (245.4) | (170.3) | (151.2) | (137.3) | (41.6) |
| SDD Formulation, | Compound 2 | 2.00 | 621 | 4250 | 4760 | 4950 | 10.4 |
| Fasted State | | (1.50-4.00) | (96.0) | (106.4) | (101.0) | (97.1) | (21.9) |

*Median (min-max)

TABLE 19
Geometric Mean (CV %) key pharmacokinetic parameters SDD formulation

| | | Cmax | MPR | AUC(0-inf) | MPR |
|---|---|---|---|---|---|
| Treatment | Analyte | (ng/mL) | Cmax | (ng · h/mL) | AUC(0-inf) |
| Cohort 3 Part2 Regimen D | Compound 1 | 218 | — | 621 | — |
| 180 mg compound 1, SDD | | (77.2) | | (77.4) | |
| Formulation, fasted state | Compound 2 | 742 | 3.41 | 5170 | 8.32 |
| | | (39.9) | (52.9) | (35.2) | (42.8) |
| Cohort 4 Regimen F | Compound 1 | 47.1 | — | 171 | — |
| 90 mg compound 1, SDD Formulation, | | (39.1) | | (53.3) | |
| Fed State (high fat breakfast) | Compound 2 | 122 | 2.60 | 901 | 5.26 |
| | | (36.1) | (42.4) | (61.5) | (34.4) |
| Cohort 5 Regimen G | Compound 1 | 111 | — | 195 | — |
| 90 mg compound 1, SDD Formulation, | | (92.2) | | (58.3) | |
| Fasted State | Compound 2 | 214 | 1.93 | 1200 | 6.16 |
| | | (44.7) | (52.0) | (52.4) | (10.5) |
| Cohort 6 Regimen H | Compound 1 | 237 | — | 836 | — |
| 270 mg compound 1, SDD | | (245.4) | | (137.3) | |
| Formulation, Fasted State | Compound 2 | 621 | 2.62 | 4950 | 5.92 |
| | | (96.0) | (69.3) | (97.1) | (32.8) |

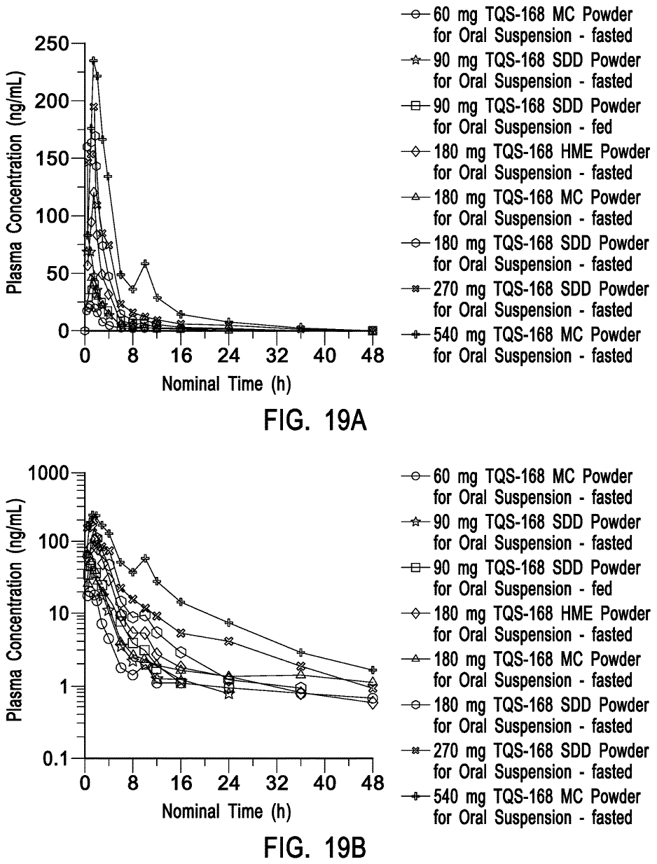

Compound 1 single ascending dose (SAD) PK profile: Cohort 3 Period 2 received Regimen D, 180 mg compound 1 SDD fonnulation in the fasted state and provided a compound 1 Cₘₐₓ of 218 ng/mL (0.87 μM). Cohort 4 received Regimen F, 90 mg compound 1 SDD formulation in the fed state and provided a compound 1 Cₘₐₓ of 47.1 ng/mL (0.19 μM). Cohort 5 received Regimen G, 90 mg compound 1 SDD formulation in the fasted state and provided a compound 1 Cₘₐₓ of 111 ng/mL (0.44 μM). Cohort 6 received Regimen H, 270 mg compound 1 SDD formulation in the fasted state and provided the highest TQS-168 Cₘₐₓ of 237 ng/mL (0.95 μM). Results are plotted in FIGS. 19A-19B. The recipients of Regimen H also displayed higher exposure (AUC₀₋₂₄=736 hr*ng/mL, AUC_{0-inf}=836 hr*ng/mL) than recipients of Regimen D (AUC₀₋₂₄=608 hr*ng/mL, AUC_{0-inf}=621 hr*ng/mL), Regimen G (AUC₀₋₂₄=192 hr*ng/mL, AUC_{0-inf}=195 hr*ng/mL) and Regimen F (AUC₀₋₂₄=168 hr*ng/mL, AUC_{0-inf}=171 hr*ng/mL).

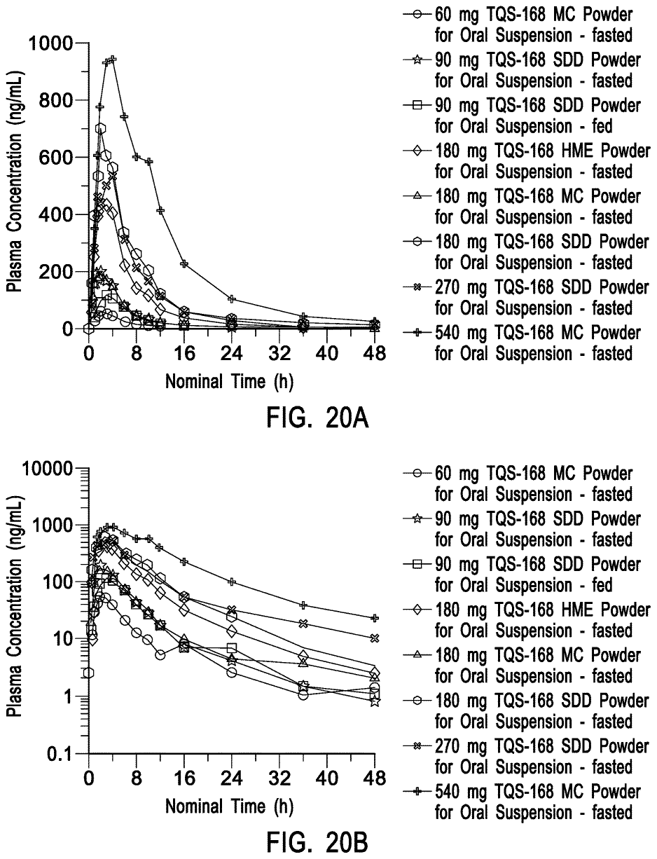

Metabolite compound 2 PK profile: Regimen D (180 mg compound 1) provided higher metabolite compound 2 Cₘₐₓ (742 ng/nL, 2.79 μM) than Regimen H (621 ng/mL, 2.33 μM), Regimen G (214 ng/mL, 0.80 μM) and Regimen F (122 ng/mL, 0.46 μM). Results are plotted in FIGS. 20A-20B. Similarly, recipients of Regimen D also displayed higher metabolite exposure (AUC₀₋₂₄=5110 hr*ng/mL, AUC_{0-inf}=5170 hr*ng/mL) than recipients of Regimen H (AUC₀₋₂₄=4250 hr*ng/mL, AUC_{0-inf}=4950 hr*ng/mL), Regimen G (AUC₀₋₂₄₌₁₁₇₀ hr*ng/mL, AUC_{0-inf}=1200 hr*ng/mL) and Regimen F (AUC₀₋₂₄=848 hr*ng/mL, AUC_{0-inf}=901 hr*ng/mL).

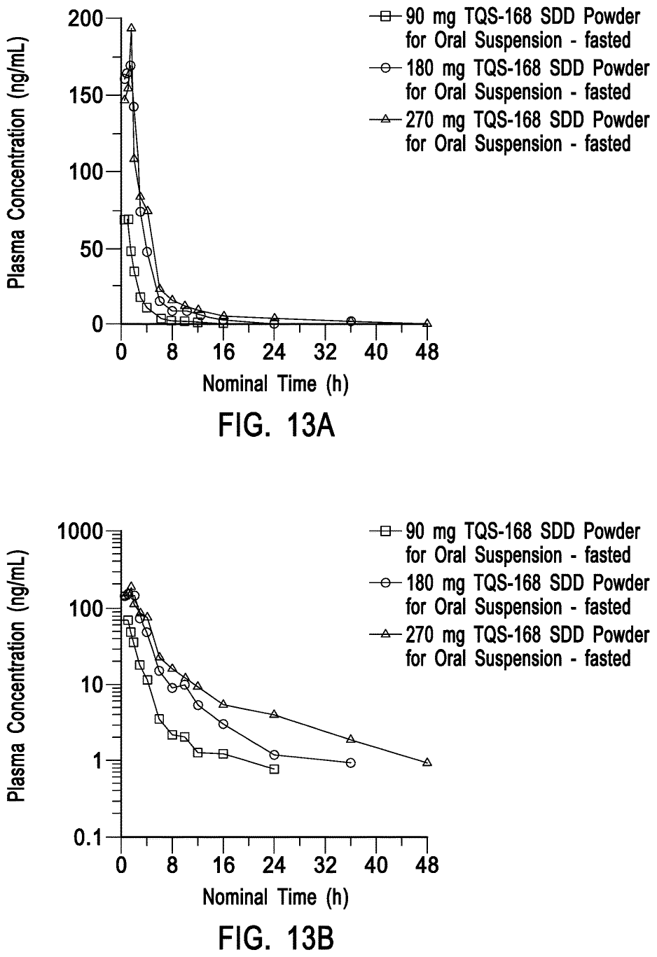

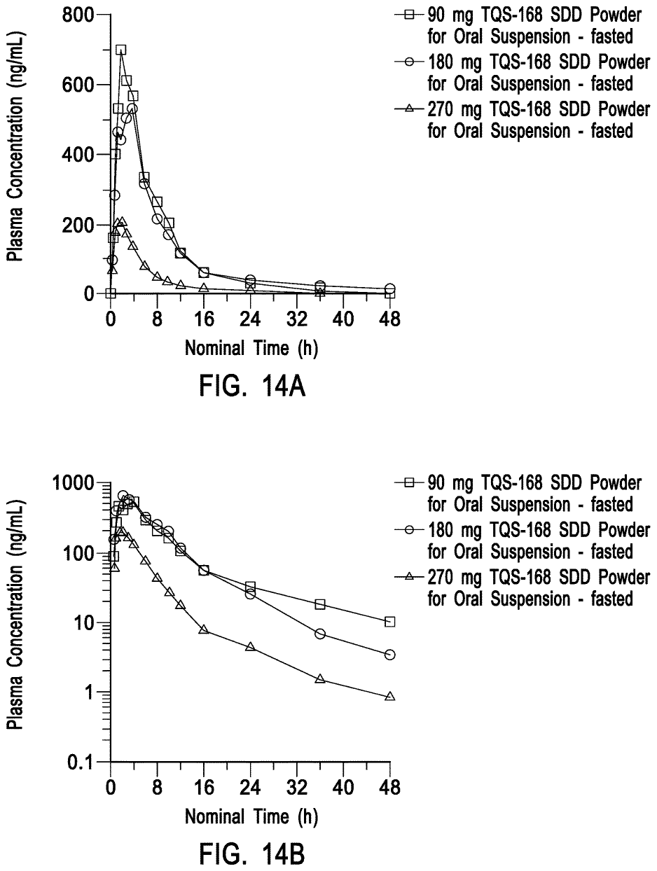

Comparing administration of 270 mg compound 1 SDD formulation in the fasted state (Regimen H) to administration of 90 mg compound 1 SDD formulation also in the fasted state (Regimen G) revealed that the 3-fold increase in dose resulted in 2.14- and 4.29-fold increases in compound 1 Cₘₐₓ and AUC_{0-inf}), and 2.90- and 4.13-fold increases in compound 2 Cₘₐₓ and AUC(_{0-inf}). See FIGS. 13A-14B.

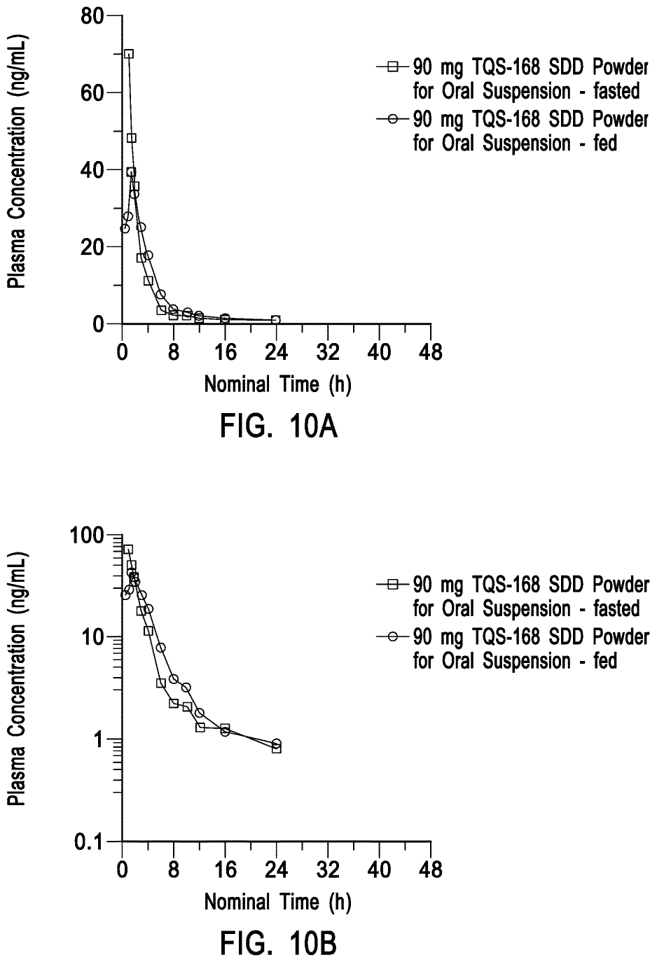

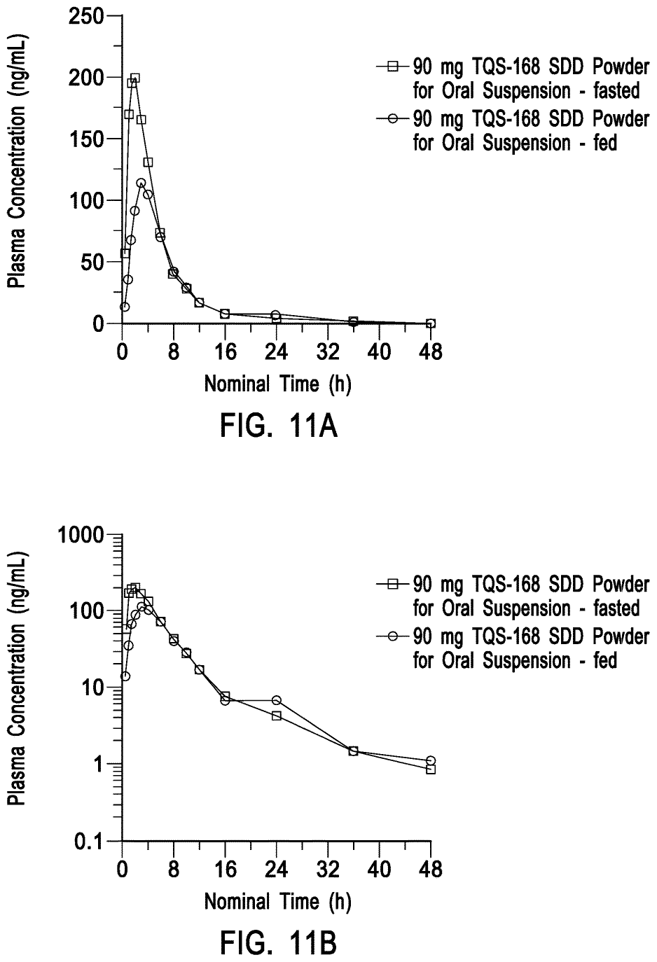

In a notable comparison, administration of 90 mg compound 1 SDD formulation in the fasted state (Regimen G) versus the same in the fed state (Regimen F), administration in the fasted state revealed increases of approximately 136% and 14% compound 1 Cₘₐₓ and AUC_{(0-inf)}, and 75% and 33% compound 2 Cₘₐₓ and AUC_{(0-inf)}. See FIGS. 10A-11B.

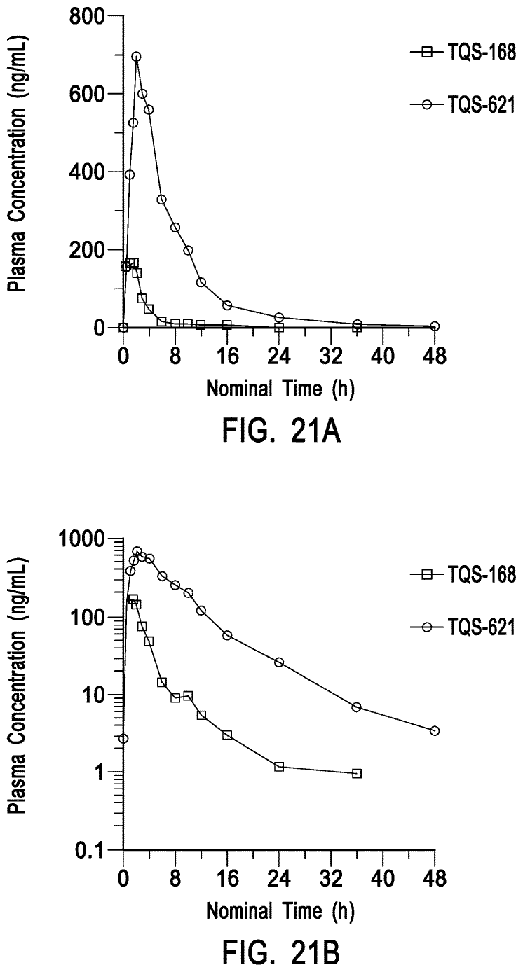

Comparing administration of 270 mg compound 1 SDD formulation in the fasted state (Regimen H) to administration of 180 mg compound 1 SDD formulation also in the fasted state (Regimen D) revealed an increase of approximately 8.7% and 26% compound 1 Cₘₐₓ and AUC_{(0-inf)}, but a decrease of approximately 16% and 4.3% compound 2 Cₘₐₓ and AUC_{(0-inf)}. See FIGS. 13A-13B and 21A-21B

Following oral administration of Regimen D, 180 mg compound 1 SDD formulation in the fasted state, maximum plasma compound 1 concentrations occurred between 0.5 and 2 hours post-dose, with a median Tₘₐₓ of 1 hour post-dose. Maximum plasma metabolite compound 2 concentrations occurred between 1.5-3.0 hours post dose, with a median Tₘₐₓ of 2 hours post-dose. See FIGS. 21A-21B.

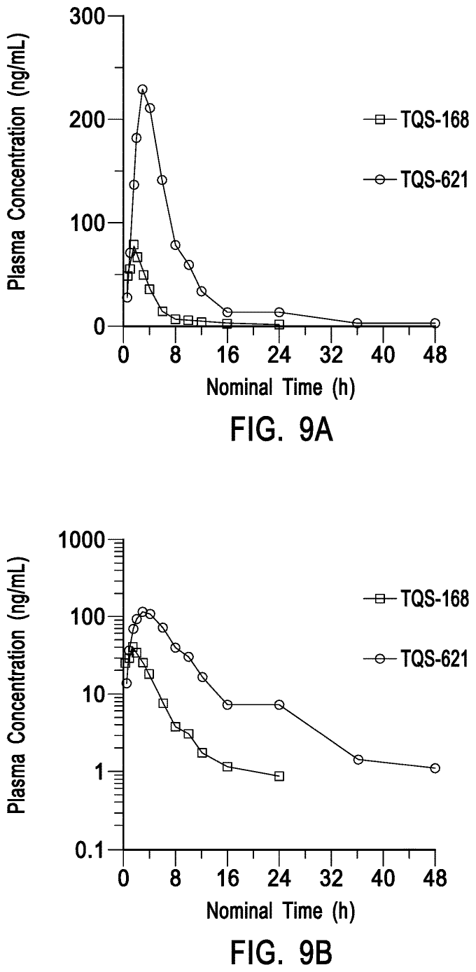

Following oral administration of Regimen F, 90 mg compound 1 SDD formulation in the fed state, maximum plasma compound 1 concentrations occurred between 0.5 and 3 hours post-dose, with a median Tₘₐₓ of 1.5 hour post-dose. Maximum plasma metabolite compound 2 concentrations occurred between 1.5-6.0 hours post dose, with a median Tₘₐₓ of 3.5 hours post-dose. See FIGS. 9A-9B.

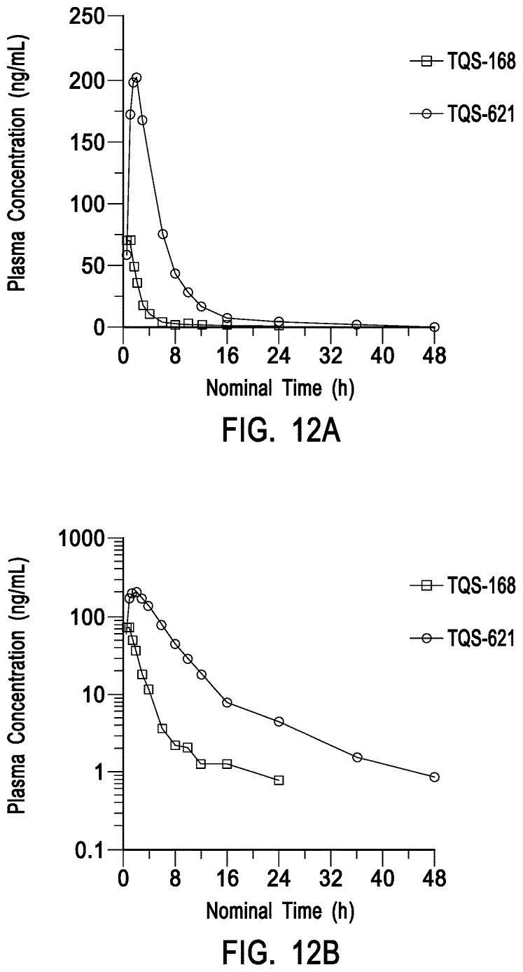

Following oral administration of Regimen G, 90 mg compound 1 in the fasted state, the maximum plasma concentrations of compound 1 occurred between 0.5 and 2.0 hours post-dose. A median Tₘₐₓ of 0.5 hour post-dose was observed. Maximum plasma metabolite compound 2 concentrations occurred between 1.0 and 3.0 hours post dose, with a median Tₘₐₓ of 1.25 hours post-dose. See FIGS. 12A-12B.

Following oral administration of Regimen H, 270 mg compound 1 SDD formulation in the fasted state, the maximum plasma concentration of compound 1 occurred between 0.5 and 4 hours post-dose, with a median Tₘₐₓ of 1.50 hours post-dose. Metabolite compound 2 displayed maximum plasma concentrations between 1.5 and 4 hours post dose, with a median Tₘₐₓ of 2 hours post-dose. See FIGS. 13A-14B.

The terminal half-life (T_{1/2}) of compound 1 following the 90 mg fed state, 90 mg fasted state, 180 mg fasted state and 270 mg fasted state compound 1 SDD formulations was dose-dependent at 3.52, 4.85, 5.64 and 10.4 hours, respectively. The terminal half-life of compound 1 metabolite compound 2 at dose 90 mg fed state, 90 mg fasted state, 180 mg fasted state and 270 mg in the fasted state was 5.17, 4.79, 7.12 and 10.4 hours, respectively. It is noted that for all doses of compound 1, plasma concentrations of compound 1 were quantifiable from 0.5 hours post-dose and remained quantifiable up to the final sample time point of 48 hours post-dose. Concentrations of compound 2 were also quantifiable from 0.5 hours post-dose and remained quantifiable up to the final sampling time point of 48 hours post-dose.

### HME Formulation Results

Healthy male subjects were administered a single oral dose of the HME formulation at 180 mg of compound 1 in the fasted state (e.g., according to Regimen E, table 14). Plasma concentrations of compound 1 and metabolite compound 2 were measured over time, and key pharmacokinetic parameters determined.

Table 20 and Table 21 present the geometric mean of compound 1 and metabolite compound 2 key pharmacokinetic parameters in the subjects following oral administration of compound 1 HME formulation.

TABLE 20
Geometric Mean (CV %) key pharmacokinetic parameters HME formulation

| | | Tmax* | Cmax | AUC(0-24) | AUC(0-last) | AUC(0-inf) | T_{1/2} |
|---|---|---|---|---|---|---|---|
| Treatment | Analyte | (h) | (ng/mL) | (ng · h/mL) | (ng · h/mL) | (ng · h/mL) | (h) |
| Cohort 3 Period 2 | Compound 1 | 1.50 | 123 | 358 | 360 | 370 | 7.25 |
| Regimen E 180 mg | | (1.00-1.50) | (66.9) | (59.5) | (60.9) | (60.2) | (54.4) |
| compound 1, HME | Compound 2 | 2.00 | 481 | 3090 | 3250 | 3290 | 9.17 |
| formulation, fasted | | (1.50-4.00) | (50.8) | (42.8) | (42.2) | (42.2) | (32.3) |
| state | | | | | | | |

TABLE 21
Geometric Mean (CV %) key pharmacokinetic parameters HME formulation

| Treatment | | Cmax | MPR | AUC(0-inf) | MPR |
|---|---|---|---|---|---|
| Part 1 | Analyte | (ng/mL) | Cmax | (ng · h/mL) | AUC(0-inf) |
| Cohort 3 Period 2 | Compound 1 | 123 | — | 370 | — |
| Regimen E | | (66.9) | | (60.2) | |
| 180 mg compound | Compound 2 | 481 | 3.90 | 3290 | 8.88 |
| 1, HME | | (50.8) | (33.9) | (42.2) | (30.2) |
| formulation, fasted | | | | | |
| state | | | | | |

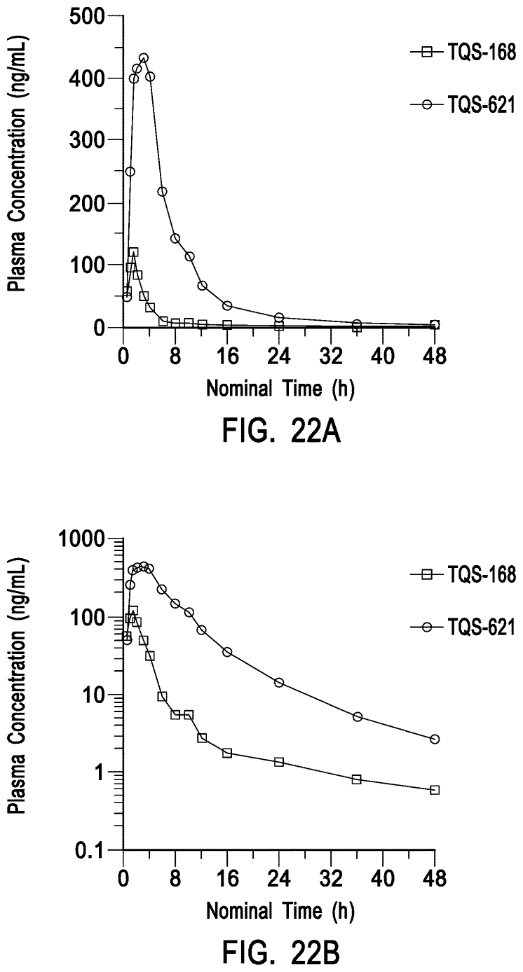

Compound 1 single ascending dose (SAD) PK profile: Cohort 3 Period 2 received Regimen E, 180 mg compound 1 HME formulation in the fasted state. The single dose provided a compound 1 Cₘₐₓ of 123 ng/mL (0.49 μM) and an AUC₀₋₂₄ of 358 hr*ng/mL. Data are plotted in FIGS. 22A-22B.

Metabolite compound 2 PK profile: Regimen E provided metabolite compound 2 Cₘₐₓ of 481 ng/mL (1.81 μM) and AUC₀₋₂₄ of 3090 hr*ng/mL. Illustrated in FIGS. 47A-B.

### Comparison of MC Formulation (Control), SDD Formulation and HME Formulation PK Results

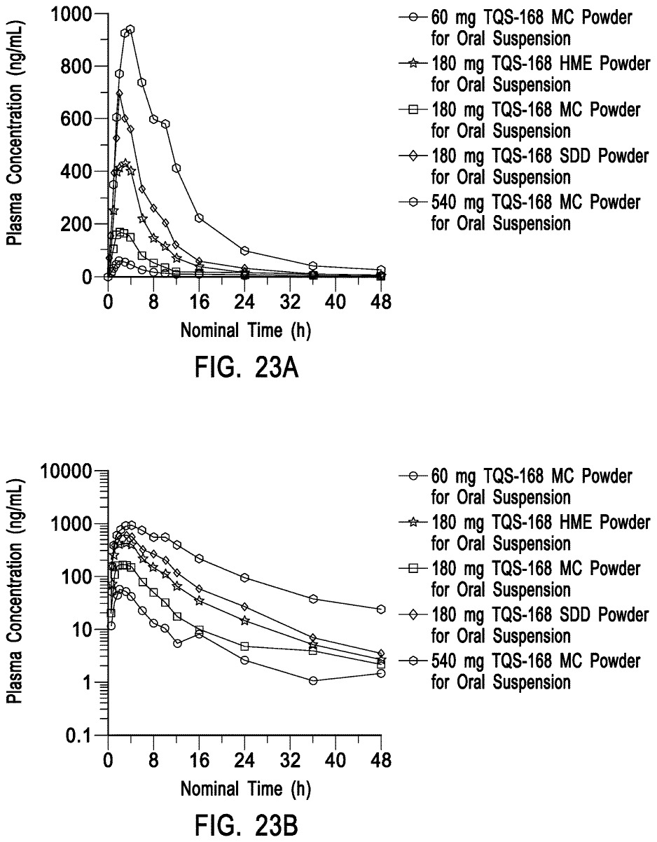

As outlined above, in this SAD trial subjects received 180 mg compound 1 in one of three separate formulations in the fasted state: MC formulation (control), SDD formulation, or HME formulation (see, e.g., tables 13 and 14). Plasma concentrations of compound 1 and metabolite compound 2 were measured over time, and key pharmacokinetic parameters determined as previously shown. For convenience, Tables 22-24 compare the results. See FIGS. 23A-23B for illustration.

TABLE 22
Geometric Mean (CV %) key pharmacokinetic parameters MC, SDD and HME formulation

| | | Tmax* | Cmax | AUC(0-24) | AUC(0-last) | AUC(0-inf) | T_{1/2} |
|---|---|---|---|---|---|---|---|
| Treatment Part 1 | Analyte | (h) | (ng/mL) | (ng · h/mL) | (ng · h/mL) | (ng · h/mL) | (h) |
| Cohort 2 Regimen B | Compound 1 | 1.00 | 53.1 | 163 | 168 | 180 | 7.36 |
| 180 mg compound 1, | | (1.00-4.00) | (55.0) | (46.3) | (51.8) | (53.1) | (75.5) |
| MC formulation | Compound 2 | 1.50 | 199 | 1210 | 1320 | 1350 | 9.10 |
| (control) | | (1.00-4.00) | (33.5) | (42.2) | (45.9) | (45.1) | (85.2) |
| Cohort 3 Regimen D | Compound 1 | 1.00 | 218 | 608 | 611 | 621 | 5.64 |
| 180 mg compound 1, | | (0.50-2.00) | (77.2) | (78.5) | (78.8) | (77.4) | (32.4) |
| SDD formulation | Compound 2 | 2.00 | 742 | 4860 | 5110 | 5170 | 7.12 |
| | | (1.50-3.00) | (39.9) | (37.3) | (35.8) | (35.2) | (32.4) |
| Cohort 3 Regimen E | Compound 1 | 1.50 | 123 | 358 | 360 | 370 | 7.25 |
| 180 mg compound 1, | | (1.00-1.50) | (66.9) | (59.5) | (60.9) | (60.2) | (54.4) |
| HME formulation | Compound 2 | 2.00 | 481 | 3090 | 3250 | 3290 | 9.17 |
| | | (1.50-4.00) | (50.8) | (42.8) | (42.2) | (42.2) | (32.3) |

*Median (min-max)

TABLE 23
Geometric Mean (CV %) key pharmacokinetic
parameters MC, SDD and HME formulation

| | | Cmax | MPR | AUC(0-inf) | MPR |
|---|---|---|---|---|---|
| Treatment | Analyte | (ng/mL) | Cmax | (ng · h/mL) | AUC(0-inf) |
| Cohort 2 | Compound 1 | 53.1 | — | 180 | — |
| Regimen B | | (55.0) | | (53.1) | |
| 180 mg | Compound 2 | 199 | 3.74 | 1350 | 7.52 |
| compound 1, | | (33.5) | (65.1) | (45.1) | (29.4) |
| MC formulation | | | | | |
| (control) | | | | | |
| Cohort 3 | Compound 1 | 218 | — | 621 | — |
| Regimen D | | (77.2) | | (77.4) | |
| 180 mg | Compound 2 | 742 | 3.41 | 5170 | 8.32 |
| compound 1, | | (39.9) | (52.9) | (35.2) | (42.4) |
| SDD | | | | | |
| formulation | | | | | |
| Cohort 3 | Compound 1 | 123 | — | 370 | — |
| Regimen E | | (66.9) | | (60.2) | |
| 180 mg | Compound 2 | 481 | 3.90 | 3290 | 8.88 |
| compound 1, | | (50.8) | (33.9) | (42.2) | (30.2) |
| HME | | | | | |
| formulation | | | | | |

TABLE 24
Geometric mean (geometric CV %) relative bioavailability of SDD powder and
HME powder for oral suspension following oral administration of TQS-168

| | | Frel | Frel | Frel | Frel | Frel | Frel |
|---|---|---|---|---|---|---|---|
| | | Cmax/D^{a} | AUC(0-last)/ | AUC(0-inf)/ | Cmax/^{b} | AUC(0-last)/^{b} | AUC(0-inf)/^{b} |
| Treatment Part 1 | Analyte | (%) | D^{a} (%) | D^{a} (%) | (%) | (%) | (%) |
| Cohort 3 Regimen D | Compound 1 | 218 | 120 | 118 | — | — | — |
| 180 mg compound 1, | | (84.9) | (80.9) | (74.6) | | | |
| SDD formulation | Compound 2 | 203 | 122 | 119 | | | |
| | | (18.8) | (56.5) | (53.3) | | | |
| Cohort 3 Regimen E | Compound 1 | 124 | 70.9 | 70.4 | 56.6 | 58.9 | 59.6 |
| 180 mg compound 1, | | (82.9) | (67.0) | (60.7) | (33.9) | (30.6) | (30.0) |
| HME formulation | Compound 2 | 132 | 77.7 | 75.7 | 64.9 | 63.5 | 63.6 |
| | | (23.2) | (50.1) | (46.3) | (16.4) | (13.8) | (13.9) |

^{a}dose normalized comparison to Regimen C: 540 mg compound 1 MC formulation
^{b}comparison to Regimen D 180 mg compound 1 SDD formulation

Subjects administered 180 mg compound 1 MC formulation (control formulation) (Regimen B) displayed plasma concentrations of compound 1 quantifiable from 0.5 hours post-dose that remained quantifiable up to between 10 and 48 hours post-dose. Concentrations of compound 2 were also quantifiable from 0.5 hours post-dose and remained quantifiable up to between 24 and 48 hours post-dose.

Maximum plasma compound 1 concentrations occurred between 1 and 4 hours post dose, with a median Tₘₐₓ of 1 hour post-dose. The resultant T_{1/2} was 7.36 hours. Geometric mean (CV %) Cₘₐₓ and AUC_{(0-inf)} values were 53.1 ng/mL (55%) and 180 ng*h/mL (53.1%) respectively.

Maximum plasma compound 2 concentrations occurred between 1 and 4 hours post dose, with a median Tₘₐₓ of 1.5 hours post-dose. The resultant geometric mean T_{1/2} was 9.10 hours. Geometric Mean (CV %) Cₘₐₓ and AUC_{(0-inf)} values were 199 ng*hr/mL (33.5%) and 1350 ng*h/mL (45.1) respectively.

Following administration of 180 mg compound 1 SDD formulation (Regimen D) plasma concentrations of compound 1 were quantifiable from 0.5 hours post-dose and remained quantifiable up to between 24 and 36 hours post-dose. Note, one subject displayed plasma concentrations of compound 1 quantifiable at pre-dose as a result of some carry-over from the previous dosing regimen. Concentration of compound 2 was quantifiable from pre-dose in all subjects as a result of some carry-over from the previous dosing regimen. It remained quantifiable up to the final sampling time point of 48 hours post-dose (Day 5). Note all quantifiable pre-dose concentrations were less than 5% of Cₘₐₓ.

Maximum plasma compound 1 concentrations occurred between 0.5 and 2 hours post-dose, with a median Tₘₐₓ of 1 hour post-dose. The resultant geometric mean T_{1/2} was 5.64 hours. Maximum plasma compound 2 concentrations occurred between 1.5 and 3 hours post-dose, with a median Tₘₐₓ of 2 hours post dose. The resultant geometric mean T_{1/2} was 7.12 hours.

The geometric mean Cₘₐₓ, AUC₍₀₋ₗₐₛₜ₎ and AUC_{(0-inf)} of compound 1 following administration of 180 mg SDD formulation compared to the MC formulation (control, Regimen B) resulted in a 4.11-, 3.64-, and 3.45-fold increase respectively. The geometric mean Cₘₐₓ, AUC₍₀₋ₗₐₛₜ₎ and AUC_{(0-inf)} of compound 2 following administration of 180 mg compound 1 SDD formulation compared to the MC formulation (control, Regimen B) resulted in a 3.73-, 3.87- and 3.83-fold increase respectively.

Following administration of 180 mg compound 1 HME formulation (Regimen E), subjects displayed plasma concentrations of compound 1 quantifiable from 0.5 hours post-dose and remained quantifiable up to between 16 and the final sampling time point of 48 hours post-dose (Day 5). Concentrations of compound 2 were also quantifiable from 0.5 hours post-dose and remained quantifiable up to the final sampling time point of 48 hours post-dose in all subjects.

Maximum plasma compound 1 concentrations occurred between 1 and 1.5 hours post-dose, with a median Tₘₐₓ of 1.5 hour post-dose. The resultant geometric mean T_{1/2}, was 7.25 hours. Maximum plasma compound 2 concentrations occurred between 1.5 and 4 hours post-dose, with a median Tₘₐₓ of 2 hour post-dose. The resultant geometric mean T_{1/2} was 9.17 hours.

The geometric mean (geometric CV %) relative bioavailability of compound 1 following administration of 180 mg compound 1 HME formulation (Regimen E), based on Cₘₐₓ, AUC₍₀₋ₗₐₛₜ₎ and AUC_{(0-inf)}, was 56.6% (33.9%), 58.9% (30.6) and 59.6% (30.0%), when compared to administration of 180 mg compound 1 SDD formulation (Regimen D). The geometric mean (geometric CV %) relative bioavailability of metabolite compound 2 following administration of 180 mg compound 1 HME formulation, based on Cₘₐₓ, AUC₍₀₋ₗₐₛₜ₎ and AUC_{(0-inf)}, was 64.9% (16.4%), 63.5% (13.8) and 63.6% (13.9)%), when compared to administration of 180 mg compound 1 SDD formulation. The geometric mean Cₘₐₓ, AUC₍₀₋ₗₐₛₜ₎ and AUC_{(0-inf)} of compound 1 following administration of 180 mg compound 1 HME formulation (Regimen E) compared to the MC formulation (control, Regimen B) resulted in a 2.32-, 2.14-, and 2.06-fold increase respectively.

The geometric mean Cₘₐₓ, AUC₍₀₋ₗₐₛₜ₎ and AUC_{(0-inf)} of compound 2 following administration of 180 mg compound 1 HME formulation compared to the MC formulation (control, Regimen B) resulted in a 2.42-, 2.46-, and 2.44-fold increase respectively.

In summary the SDD formulation showed significant improvement in exposure over the MC and the HME formulations of the same compound 1 dosage, as well as in exposure of metabolite compound 2. See FIGS. 23A-23B. In addition, the exposures to compound 1 and metabolite compound 2 were greater after dosing with both the SDD and HME formulations as compared to dosing with the MC formulation (control formulation).

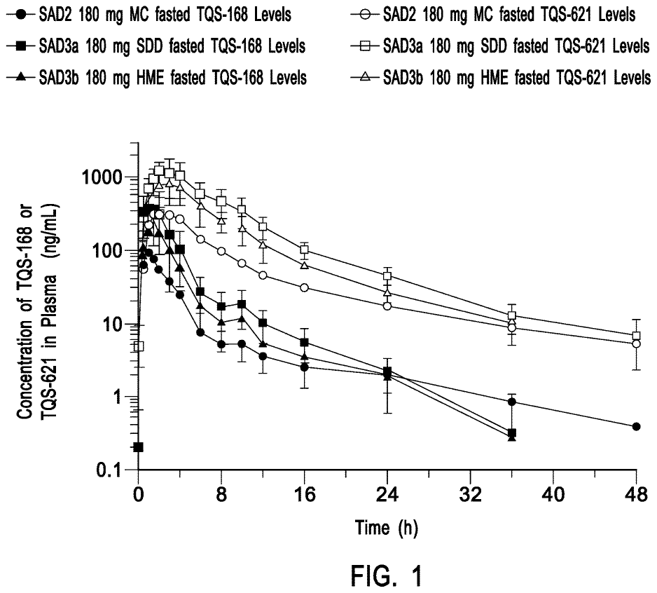

FIG. 1 shows the plasma concentrations of compound 1 and metabolite compound 2 over time after a single oral dose of 180 mg SDD formulation of compound 1; a HME formulation of compound 1; and a control formulation (compound 1 as a MC formulation) in humans.

### 6.1. Example 8: Multiple Dose Phase 1 Trial—Double-Blind Randomized Study Of Subjects Receiving Multiple Doses of TQS-168 or Placebo

A double-blind, randomized, placebo-controlled clinical study was conducted to characterize and compare the pharmacokinetic (PK) profile of compound 1 and its metabolite compound 2 following multiple oral doses of a compound 1 SDD formulation (e.g., as described in table 13) in healthy subjects. See Table 25 for description of dose regimens.

TABLE 25
Description of Regimens for Multiple oral
doses of compound 1 SDD formulation

| | Period and | | | |
|---|---|---|---|---|
| | Frequency | | | |
| Cohort | (Dosing Day) | Regimen | Dose | State |
| 1 | 1-7 QD | I | 120 mg | Fasted |
| 2 | 1-7 QD | J | 90 mg | Fed |
| 3 | 1-7 QD | K | 300 mg | Fed |

This randomized, double-blind, placebo-controlled phase 1 multiple dose trial was conducted in healthy male subjects aged 18 to 55 years with body mass index (BMI) 18.0 to 32.0 kg/m² as measured at screening. Subjects all weighed at least 55 kg at screening. Key criteria for exclusion were subjects with evidence of current SARS-CoV-2 infection, clinical manifestation of significant cardiovascular, renal, hepatic, dermatological, chronic respiratory or gastrointestinal disease, or aspartate aminotransferase (AST), or alanine aminotransferase (ALT)>1.5× the upper limit of normal (ULN). Subjects were recruited at a single site in the United Kingdom. Each patient provided written informed consent.

The trial was performed in 3 cohorts. All subjects were admitted in the morning on the day before dosing (Day −1) and remained onsite until 48 hours post-final dose (Day 9). The screening period was 4 weeks. After confirming eligibility, subjects were randomly assigned to receive either the compound 1 SDD formulation or placebo treatment. Subjects were dosed with the compound 1 SDD formulation or placebo on the morning of Days 1 to 7 (approximately 24 hours apart). Administration was performed in either the fasted state (Regimen I) following an overnight fast (minimum of 10 hours), or the fed state (following a standard pre-dose or high fat meal given 30 minutes before dosing). Safety was continually assessed throughout the trial by monitoring adverse events and concomitant medication use, electrocardiograms (ECGs), vital signs, laboratory safety assessments and physical examinations. Blood samples for pharmacokinetic assessments were collected from each subject prior from Day −1, (≤1 hr) to each dose, and at intervals throughout the study until 48 hours post final dose as applicable.

Assessment of Pharmacokinetics: Blood samples for plasma PK analysis were collected at regular time intervals. Venous blood samples were collected from the subjects by a trained member of the clinical team. Pre-dose samples were taken ≤1 h before dosing. Timestamp 0 to 1 hour post-dose samples were taken within ±2 minutes of the nominal post-dose sampling time. Timestamp 1.5 to 12 hour post-dose samples were taken within ±10 min of the nominal post-dose sampling time. Timestamp 16 to 48 hour post-dose samples were taken within ±30 minutes of the nominal post-dose sampling time. Samples were collected into appropriate containers and were processed to isolate plasma. PK analysis were carried out on plasma samples using validated bioanalytical methods.

Statistical Analyses: The sample sizes for the study were chosen based on practical considerations and experience from previous studies of a similar design. The numbers of subjects in each cohort (group) were considered to be adequate to assess the main objectives of each study. Pharmacokinetic parameters were determined by non-compartmental techniques using WinNonlin software version 8.0 or higher (Certara USA. Inc., USA). All data were listed and summarized by subject group using descriptive statistics. All statistical analyses were conducted using SAS version 9.4 or higher.

Regimen I: Subjects received an oral dose of 120 mg compound 1 SDD formulation, or placebo, once a day for 7 consecutive days in the fasted state.

Regimen J: Subjects received an oral dose of 90 mg compound 1 SDD formulation, or placebo, once a day for 7 consecutive days in the fed state. Subjects were provided a high fat breakfast on Day 1 and 7, and a standard breakfast on Days 2-6.

Regimen K: Subjects received an oral dose of 300 mg compound 1 SDD formulation, or placebo, once a day for 7 consecutive days in the fed state. Subjects were provided a high fat breakfast on Day 1 and 7, and a standard breakfast on Days 2-6.

### 6.1.1. Results

Healthy male subjects were administered multiple oral doses of either 120 mg compound 1 SDD formulation in the fasted state (Regimen I) or 90 mg compound 1 SDD formulation in the fed state (Regimen J). Plasma concentrations of compound 1 and metabolite compound 2 were measured over time, and key pharmacokinetic parameters determined.

Table 26 presents the geometric mean of compound 1 and metabolite compound 2 key pharmacokinetic parameters in the subjects following oral administration of compound 1.

TABLE 26
Geometric Mean (CV %) key pharmacokinetic parameters

| | | | | | | | MPR | MPR | AR | AR |
|---|---|---|---|---|---|---|---|---|---|---|
| | | | Tmax* | Cmax | AUC(0-tau) | T_{1/2} | Cmax | AUC(0-tau) | Cmax | AUC |
| Treatment | Analyte | Day | (h) | (ng/mL) | (ng · h/mL) | (h) | (N/A) | (N/A) | (N/A) | (N/A) |
| Cohort 1 Regimen I | Compound 1 | 1 | 1.00 | 172 | 438 | — | — | — | — | — |
| 120 mg compound 1 | | | (0.50-2.00) | (49.3) | (51.5) | | | | | |
| SDD formulation | | 7 | 0.750 | 273 | 692 | 7.70 | — | — | 1.59 | 1.58 |
| QD in the Fasted | | | (0.500-1.50) | (105.8) | (89.8) | (39.0) | | | (88.1) | (51.6) |
| State | Compound 2 | 1 | 1.50 | 331 | 2270 | — | 1.93 | 5.19 | — | — |
| | | | (1.00-4.00) | (40.3) | (35.7) | | (32.7) | (26.1) | | |
| | | 7 | 1.75 | 340 | 3320 | 10.2 | 1.24 | 4.79 | 1.03 | 1.46 |
| | | | (1.00-4.00) | (39.5) | (42.1) | (35.5) | (57.3) | (40.4) | (38.7) | (30.8) |
| Cohort 2 Regimen J | Compound 1 | 1 | 3.00 | 47.4 | 223 | — | — | — | — | — |
| 90 mg compound 1 | | | (1.50-4.00) | (27.4) | (30.9) | | | | | |
| SDD formulation | | 7 | 3.00 | 48.8 | 227 | 4.46 | — | — | 1.03 | 1.24 |
| QD in the Fed State | | | (1.00-4.00) | (48.3) | (46.7) | (36.5) | | | (37.5) | (22.8) |
| | Compound 2 | 1 | 4.00 | 189 | 1400 | — | 3.99 | 6.28 | — | — |
| | | | (4.00-6.00) | (30.1) | (42.6) | | (26.6) | (20.6) | | |
| | | 7 | 5.00 | 218 | 2060 | 8.65 | 4.47 | 7.43 | 1.15 | 1.47 |
| | | | (4.00-6.00) | (37.6) | (54.8) | (10.3 | (14.9) | (10.7) | (12.4) | (18.0) |
| Cohort 3 Regimen K | Compound 1 | 1 | 2.00 | 229 | 1210 | — | — | — | — | — |
| 300 mg compound 1 | | | (1.50-4.00) | (38.3) | (55.1) | | | | | |
| SDD formulation | | 7 | 4.00 | 400 | 2010 | 5.67 | — | — | 1.74 | 1.66 |
| QD in the fed state | | | (0.50-4.00) | (79.3) | (88.3) | (52.2) | | | (54.6) | (29.5) |
| | Compound 2 | 1 | 4.00 | 1000 | 9730 | — | 4.37 | 8.06 | — | — |
| | | | (4.00-10.00) | (24.6) | (39.9) | | (26.1) | (18.1) | | |
| | | 7 | 5.00 | 1300 | 14900 | 7.29 | 3.26 | 7.41 | 1.30 | 1.53 |
| | | | (4.00-6.00) | (36.7) | (61.2) | (42.4) | (43.4) | (25.5) | (32.9) | (26.1) |

*Median (min-max)
Tau = 24 h
N/A = not applicable

Compound 1 Regimen I PK profile: Subjects in Cohort 1 received a single 120 mg compound 1 SDD formulation p.o. QD in the fasted state for seven consecutive days.

On Day 1, following a single administration of Regimen I, plasma concentrations of compound 1 were quantifiable from 0.5 hours post-dose for all subjects, and remained quantifiable up to between 16 and 24 hours post-dose. Concentrations of compound 2 on Day 1 were also quantifiable from 0.5 h post-dose, and remained quantifiable up to 24 hours post-dose in all subjects.

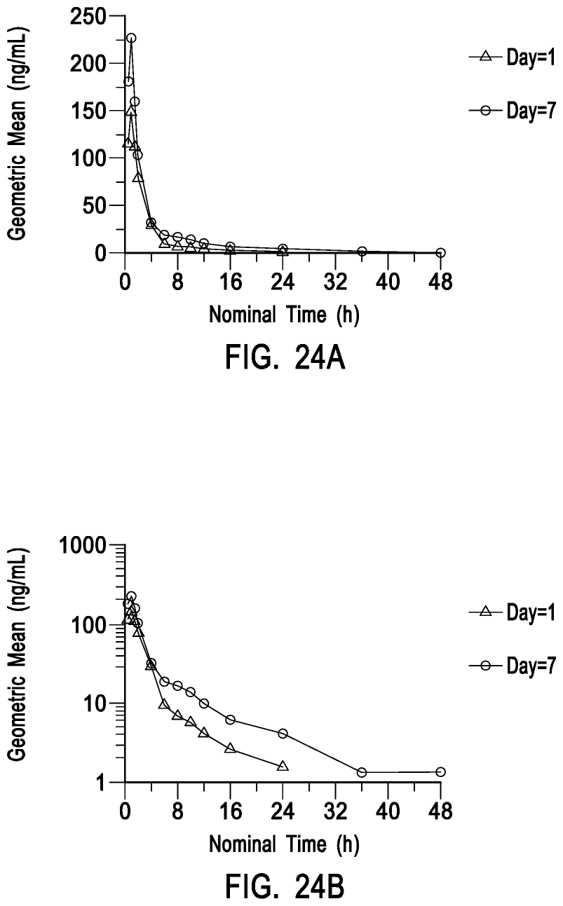

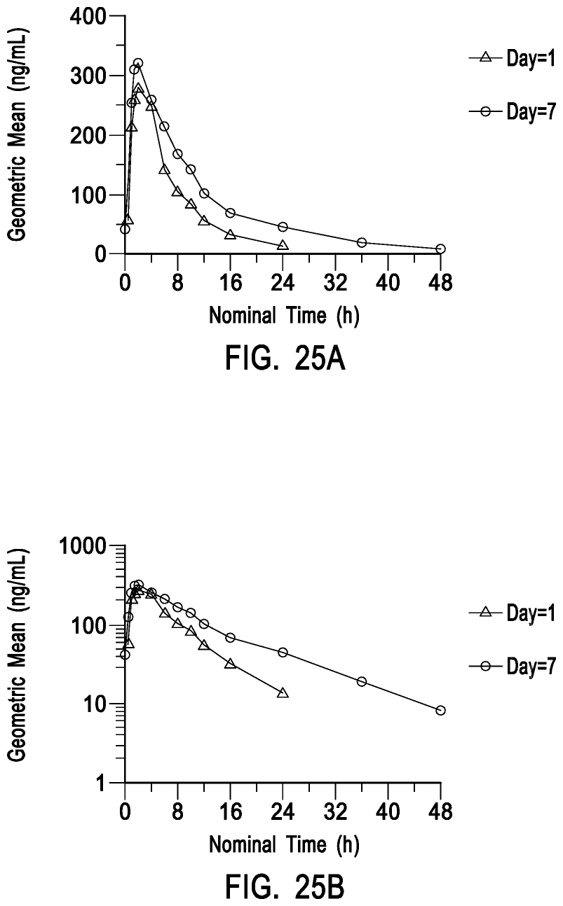

Maximum plasma compound 1 concentrations on Day 1 occurred between 0.5 and 2.0 hours post-dose, with a median Tₘₐₓ of 1.0 hours post-dose. Geometric mean (CV %) Cₘₐₓ and AUC₍₀₋ₜₐᵤ₎ values 172 ng/mL (49.3%) and 438 ng*h/mL (55.1%), respectively. Maximum plasma compound 2 concentrations on Day 1 occurred between 1.0 and 4.0 hours post-dose, with a median Tₘₐₓ of 1.5 hours post-dose. Geometric mean (CV %) Cₘₐₓ and AUC₍₀₋ₜₐᵤ₎ values 331 ng/mL (40.3%) and 2270 ng*h/mL (35.7%), respectively. See FIGS. 24A-25B.

Following multiple administrations, Day 7 plasma concentrations of compound 1 and metabolite compound 2 were quantifiable at pre-dose in all but one subject who became quantifiable at 0.5 hours post dose. The subjects all remained quantifiable up to between 16 and the final sampling time point of 48 hours post-dose.

Maximum plasma compound 1 concentrations on Day 7 occurred between 0.5 and 1.5 hours post-dose, with a median Tₘₐₓ of 0.75 hours post-dose. Concentrations then declined yielding a mean elimination half-life of 7.7 hours. Geometric mean (CV %) Cₘₐₓ and AUC₍₀₋ₜₐᵤ₎ values were 273 ng/mL (105.8%) and 692 ng*h/mL (89.8%), respectively. Geometric mean (CV %) accumulation ratios were 1.59 (88.1%) and 1.58 (51.6%), based on Cₘₐₓ and AUC₍₀₋ₜₐᵤ₎, respectively. See FIGS. 24A-25B.

Maximum plasma compound 2 concentrations on Day 7 occurred between 1.0 and 4.0 hours post-dose, with a median Tₘₐₓ of 1.75 hours post-dose. Concentrations then declined yielding a mean elimination half-life of 10.2 hours. Geometric mean (CV %) Cₘₐₓ and AUC₍₀₋ₜₐᵤ₎ values were 340 ng/mL (39.5%) and 3320 ng*h/mL (42.1%), respectively. Geometric mean (CV %) accumulation ratios were 1.30 (38.7%) and 1.46 (30.8%), based on Cₘₐₓ and AUC₍₀₋ₜₐᵤ₎, respectively. See FIGS. 24A-25B.

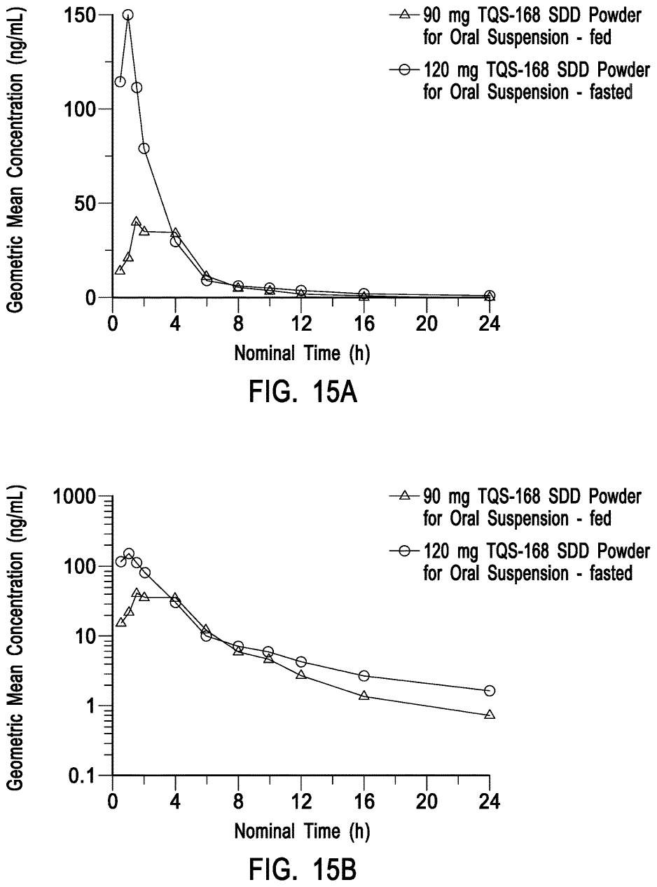

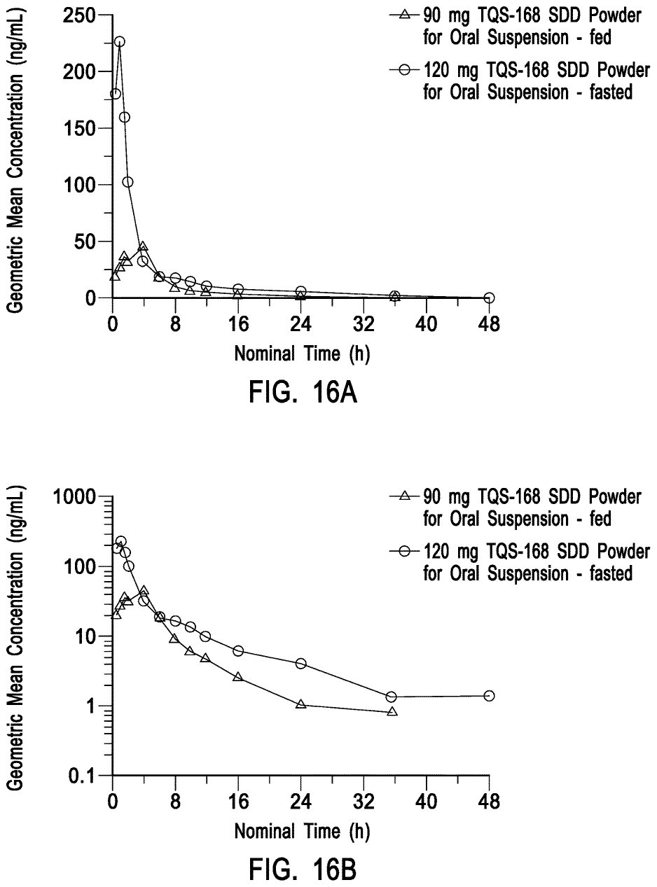

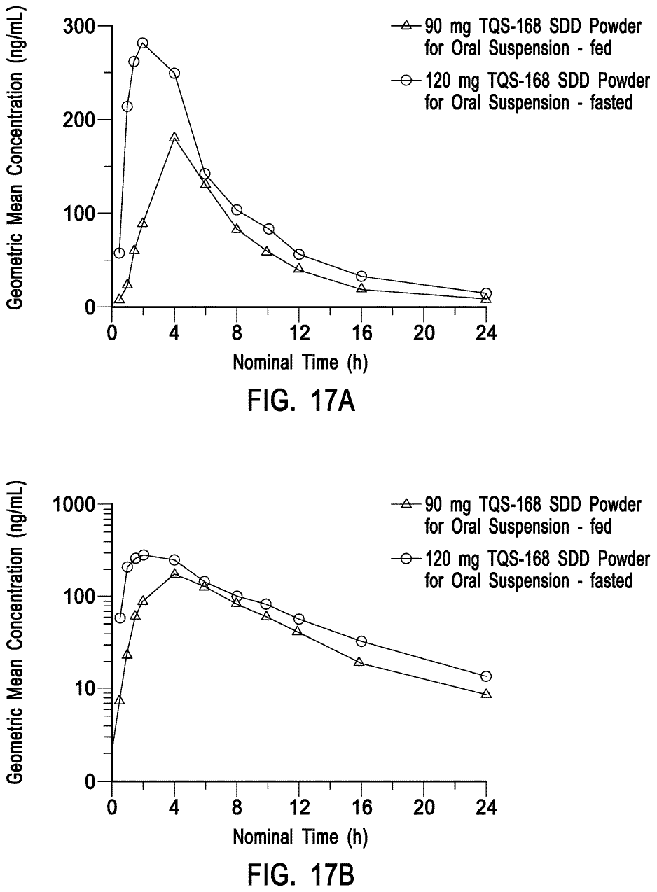

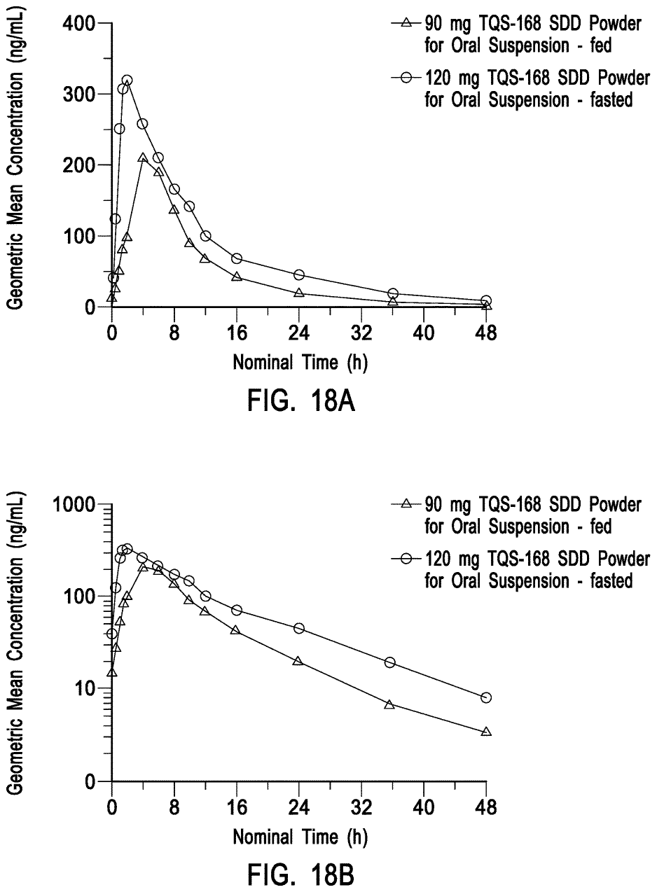

Regimen I PK summary: Subjects in Cohort 1 were administered Regimen I and received 120 mg compound 1 SDD formulation QD for seven consecutive days in the fasted state. Day 1 provided a compound 1 Cₘₐₓ of 172 ng/mL (0.69 μM). Day 7 of consecutive dosing provided a much greater compound 1 Cₘₐₓ of 273 ng/mL (1.09 μM). Results are plotted in FIGS. 15A-16B. Similar increases were observed in AUC measured exposure (Day 1 AUC₍₀₋ₜₐᵤ₎=438 hr*ng/mL, Day 7 AUC₍₀₋ₜₐᵤ₎=692 hr*ng/mL). Metabolite compound 2 PK profile: Cohort 1 displayed a Day 1 metabolite compound 2 Cₘₐₓ of 331 ng/mL (1.24 μM) and a Day 7 Cₘₐₓ of 340 ng/mL (1.27 μM). Results are plotted in FIGS. 17A-18B. A metabolite exposure of AUC₍₀₋ₜₐᵤ₎=2270 hr*ng/mL was recorded for Day 1. Day 7 provided a metabolite exposure of AUC₍₀₋ₜₐᵤ₎=3220 hr*ng/m. See FIGS. 24A-25B.

Compound 1 Regimen J PK profile: Subjects in Cohort 2 received a single 90 mg compound 1 SDD formulation p.o. QD in the fed state for seven consecutive days.

On Day 1, following a single administration of 90 mg compound 1 SDD formulation, plasma concentrations of compound 1 were quantifiable from 0.5 hours post-dose for all subjects, and remained quantifiable up to between 16 and 24 hours post-dose. Concentrations of compound 2 on Day 1 were also quantifiable from 0.5 h post-dose and remained quantifiable up to 24 hours post-dose in all subjects.

Maximum plasma compound 1 concentrations on Day 1 occurred between 1.5 and 4.0 hours post-dose, with a median Tₘₐₓ of 3.0 hours post-dose. Geometric mean (CV %) Cₘₐₓ and AUC₍₀₋ₜₐᵤ₎ values were 47.4 ng/mL (27.4%) and 223 ng.h/mL (30.9%), respectively. Comparison with Regimen I illustrated in FIGS. 15A-15B.

Maximum plasma compound 2 concentrations on Day 1 occurred between 4.0 and 6.0 hours post-dose, with a median Tₘₐₓ of 4.0 hours post-dose. Geometric mean (CV %) Cₘₐₓ and AUC₍₀₋ₜₐᵤ₎ values were 189 ng/mL (40.1%) and 1400 ng*h/mL (42.6%), respectively. Comparison with Regimen I illustrated in FIGS. 17A-17B.

On Day 1, no individual subject exceeded the maximum permitted Cₘₐₓ or the maximum permitted AUC₍₀₋₂₄₎ (based AUC₍₀₋ₜₐᵤ₎, where tau=24 h) values. The maximum individual compound 1 Cₘₐₓ on Day 1 was 65.8 ng/mL, which accounted for 21.6% of the Cₘₐₓ exposure limit. The maximum individual compound 1 AUC₍₀₋ₜₐᵤ₎ was 299 ng*h/mL, which accounted for 10.9% of the AUC₍₀₋ₜₐᵤ₎ exposure limit.

Following multiple administrations of 90 mg compound 1 SDD formulation to healthy subjects in the fed state for 7 days, plasma concentrations of compound 1 were quantifiable at pre-dose in all but three subjects who became quantifiable at 0.5 hours post-dose, and remained quantifiable up to between 16 and 36 hours post-dose. Concentrations of compound 2 were quantifiable at the pre-dose time-point in all subjects, and remained quantifiable up to the final sampling time point of 48 hours post-dose.

Maximum plasma compound 1 concentrations on Day 7 occurred between 1.0 and 4.0 hours post-dose, with a median Tₘₐₓ of 3.0 hour post-dose. geometric mean elimination half-life of 4.46 h. Geometric mean (CV %) Cₘₐₓ and AUC₍₀₋ₜₐᵤ₎ values were 48.8 ng/mL (48.3%) and 227 ng.h/mL (46.7%) respectively. Geometric mean (CV %) accumulation ratios were 1.03 (37.5%) and 1.24 (22.8%), based on Cₘₐₓ and AUC₍₀₋ₜₐᵤ₎, respectively. Comparison with Regimen I illustrated in FIGS. 16A-16B.

Maximum plasma compound 2 concentrations on Day 7 occurred between 4.0 and 6.0 hours post-dose, with a median Tₘₐₓ of 5.0 hours post-dose. Concentrations then declined in a generally biphasic manner giving rise to a geometric mean elimination half-life of 8.65 h. Geometric mean (CV %) Cₘₐₓ and AUC₍₀₋ₜₐᵤ₎ values were 218 ng/mL (37.6%) and 2060 ng.h/mL (54.8%) respectively. Geometric mean (CV %) accumulation ratios were 1.15 (12.4%) and 1.47 (18.0%), based on Cₘₐₓ and AUC₍₀₋ₜₐᵤ₎, respectively. Comparison with Regimen I illustrated in FIGS. 18A-18B.

On Day 7, no individual subjects exceeded the maximum permitted Cₘₐₓ value for compound 1 or the maximum permitted AUC₍₀₋₂₄₎ (based AUC₍₀₋ₜₐᵤ₎, where tau=24 h values. The maximum individual compound 1 Cₘₐₓ on Day 7 was 94.1 ng/mL, which accounted for 30.9% of the Cₘₐₓ exposure limit. The maximum individual compound 1 AUC₍₀₋ₜₐᵤ₎ was 535 ng.h/mL, which accounted for 19.5% of the AUC₍₀₋₂₄₎ exposure limit.

Regimen J PK summary: subjects in Cohort 2 were administered Regimen J and received a once a day dose of 90 mg compound 1 SDD formulation in the fed state and provided a Day 1 compound 1 Cₘₐₓ of 47.4 ng/mL (0.19 μM). On Day 7 of consecutive dosing with 90 mg compound 1 SDD formulation in the fed state, a compound 1 Cₘₐₓ of 48.8 ng/mL (0.19 μM) was observed. Results are plotted in FIGS. 15A-16B. Similar figures were observed in comparison of the exposure profile (Day 1 AUC₍₀₋ₜₐᵤ₎=223 hr*ng/mL, Day 7 AUC₍₀₋ₜₐᵤ₎=227 hr*ng/mL. Regarding compound 2 PK profile, Cohorts displayed a Day 1 metabolite compound 2 Cₘₐₓ of 189 ng/mL (0.71 μM) and a Day 7 Cₘₐₓ of 218 ng/mL (0.82 μM). Results are plotted in FIGS. 17A-18B. A metabolite exposure of AUC₍₀₋ₜₐᵤ₎=1400 hr*ng/mL was recorded for Day 1. Day 7 provided a metabolite exposure of AUC₍₀₋ₜₐᵤ₎=2060 hr*ng/m.

Compound 1 Regimen K PK profile: Subjects in Cohort 3 received a single 300 mg compound 1 SDD formulation QD in the fed state for seven consecutive days.

On Day 1, following a single administration of 300 mg compound 1 SDD formulation, plasma concentrations of compound 1 were quantifiable from 0.5 hours post-dose for all subjects, and remained quantifiable up to the final sampling point of 24 h post dose in all subjects.

Maximum plasma compound 1 concentrations on Day 1 occurred between 1.5 and 4.0 hours post-dose with a median Tₘₐₓ of 2.0 hours post-dose. Geometric mean (CV %) Cₘₐₓ and AUC₍₀₋ₜₐᵤ₎ values were 229 ng/mL (38.3%) and 1210 ng*h/mL (55.1), respectively. See FIGS. 51A-B for illustrated comparison with Regimen I and J.

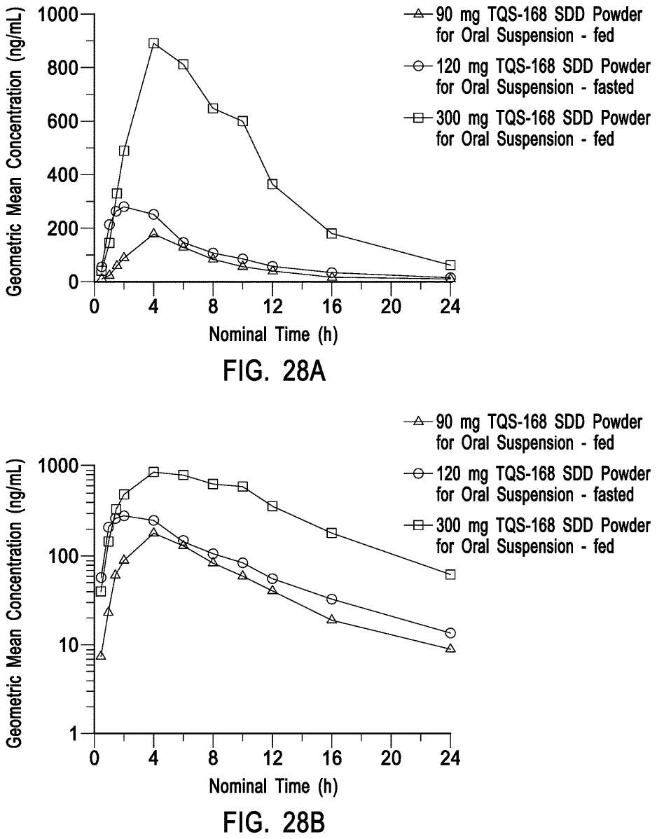

Maximum plasma compound 2 concentrations on Day 1 occurred between 4.0 and 10.0 hours post-dose, with a median Tₘₐₓ of 4.0 hours post-dose. Geometric mean (CV %) Cₘₐₓ and AUC₍₀₋ₜₐᵤ₎ values 1000 ng/mL (24.6%) and 9730 ng*h/mL (39.9%), respectively. See FIGS. 28A-28B for illustrated comparison with Regimen I and J.

Following multiple administrations Day 7 plasma concentrations of compound 1 and metabolite compound 2 were quantifiable at pre-dose in all subjects and remained quantifiable up to between 24 and 48 hours post-dose. Concentrations of compound 1 were also quantifiable at the pre-dose time-point in all subjects and remained quantifiable up the final sampling time point of 48 hours post-dose.

Maximum plasma compound 1 concentrations on Day 7 occurred between 0.50 and 4.0 hours post-dose, with a median Tₘₐₓ of 4.0 hours post-dose. Concentrations then declined yielding a mean elimination half-life of 5.67 hours. Geometric mean (CV %) Cₘₐₓ and AUC₍₀₋ₜₐᵤ₎ values were 400 ng/mL (79.3%) and 2010 ng*h/mL (88.3%), respectively. Geometric mean (CV %) accumulation ratios were 1.74 (54.6%) and 1.66 (29.5%), based on Cₘₐₓ and AUC₍₀₋ₜₐᵤ₎, respectively. See FIGS. 52A-B for illustrated comparison with Regimen I and J.

Maximum plasma compound 2 concentrations on Day 7 occurred between 4.0 and 6.0 hours post-dose, with a median Tₘₐₓ of 5.0 hours post-dose. Concentrations then declined yielding a mean elimination half-life of 7.29 hours. Geometric mean (CV %) Cₘₐₓ and AUC₍₀₋ₜₐᵤ₎ values were 1300 ng/mL (36.7%) and 14900 ng*h/mL (61.2%), respectively. Geometric mean (CV %) accumulation ratios were 1.30 (32.9%) and 1.53 (26.1%), based on Cₘₐₓ and AUC₍₀₋ₜₐᵤ₎, respectively. See FIGS. 54A-B for illustrated comparison with Regimen I and J.

Regimen K PK summary, subjects in Cohort 1 were administered Regimen K and received 300 mg compound 1 SDD formulation QD for seven consecutive days in the fed state. Day 1 provided a compound 1 Cₘₐₓ of 229 ng/mL (0.91 μM). Day 7 of consecutive dosing provided a much greater compound 1 Cₘₐₓ of 400 ng/mL (1.60 μM). Results are plotted in FIGS. 15A-16B. Similar increases were observed in AUC measured exposure (Day 1 AUC₍₀₋ₜₐᵤ₎=1210 hr*ng/mL, Day 7 AUC₍₀₋ₜₐᵤ₎=2010 hr*ng/mL). Cohort 1 displayed a Day 1 metabolite compound 2 Cₘₐₓ of 1000 ng/mL (3.76 μM) and a Day 7 Cₘₐₓ of 1300 ng/mL (4.88 μM). Results are plotted in FIGS. 17A-18B. A metabolite exposure of AUC₍₀₋ₜₐᵤ₎=9730 hr*ng/mL was recorded for Day 1. Day 7 provided a metabolite exposure of AUC₍₀₋ₜₐᵤ₎=14900 hr*ng/m.

In summary, this trial provided healthy male subjects with consecutive seven QD doses of compound 1 in the fed or fasted state at different dosages of compound 1 SDD formulation (e.g., in accordance with Table 13 SDD formulation). The data reveals that increase in dosage corresponds to an increase in plasma concentration of compound 1 and compound 1 metabolite compound 2.

### 7. EQUIVALENTS AND INCORPORATION BY REFERENCE

While the disclosure has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. An amorphous solid dispersion comprising 2-(4-tert-butylphenyl)-1H-benzimidazole (compound 1) or a pharmaceutically acceptable salt thereof; and a pharmaceutically acceptable carrier matrix.

2. The amorphous solid dispersion according to claim 1, prepared by spray-drying or hot-melt extrusion.

3. The amorphous solid dispersion according to claim 2, prepared by spray-drying.

4. The amorphous solid dispersion according to claim 2, prepared by hot-melt extrusion.

5. The amorphous solid dispersion of any one of claims 1 to 4, comprising 40% w/w or less of 2-(4-tert-butylphenyl)-1H-benzimidazole (compound 1) or a pharmaceutically acceptable salt thereof.

6. The amorphous solid dispersion of claim 5, comprising 30% w/w of 2-(4-tert-butylphenyl)-1H-benzimidazole (compound 1) or a pharmaceutically acceptable salt thereof.

7. The amorphous solid dispersion of claim 5, comprising 25% w/w of 2-(4-tert-butylphenyl)-1H-benzimidazole (compound 1) or a pharmaceutically acceptable salt thereof.

8. The amorphous solid dispersion of any one of claims 1 to 7, wherein the pharmaceutically acceptable carrier matrix comprises a polyvinyl caprolactam-polyvinyl acetate-polyethylene glycol graft co-polymer, a polyvinylpyrrolidone polymer, a copovidone polymer, a povidone polymer, a hydroxypropyl methyl cellulose polymer, a dimethylaminoethyl methacrylate-copolymer, a methacrylic acid-methyl methacrylate copolymer, a polyethylene glycol polymer, amorphous silicon dioxide and mixtures thereof.

9. The amorphous solid dispersion of claim 8, wherein the pharmaceutically acceptable carrier matrix comprises a copovidone polymer, a povidone polymer, a polyvinyl caprolactam-polyvinyl acetate-polyethylene glycol graft co-polymer, amorphous silicon dioxide, or a mixture thereof.

10. The amorphous solid dispersion of claim 8 or 9, wherein the pharmaceutically acceptable carrier matrix comprises a mixture of a polyvinyl caprolactam-polyvinyl acetate-polyethylene glycol graft co-polymer, and amorphous silicon dioxide.

11. The amorphous solid dispersion of claim 8 or 9, wherein the pharmaceutically acceptable carrier matrix comprises a mixture of copovidone polymer, a povidone polymer, and a polyvinyl caprolactam-polyvinyl acetate-polyethylene glycol graft co-polymer.

12. The amorphous solid dispersion of any one of claims 1 to 11, comprising 60 to 85% w/w of the pharmaceutically acceptable carrier matrix.

13. The amorphous solid dispersion of claim 12, comprising 65 to 80% w/w of the pharmaceutically acceptable carrier matrix.

14. The amorphous solid dispersion of claim 13, comprising 70% w/w of the pharmaceutically acceptable carrier matrix.

15. The amorphous solid dispersion of claim 13, comprising 75% w/w of the pharmaceutically acceptable carrier matrix.

16. An amorphous solid dispersion comprising:
10-40% w/w of 2-(4-tert-butylphenyl)-1H-benzimidazole (compound 1) or a pharmaceutically acceptable salt thereof; and
60 to 90% w/w of a pharmaceutically acceptable carrier matrix,
wherein the amorphous solid dispersion is prepared by spray-drying.

17. The amorphous solid dispersion of claim 16, comprising:
30% w/w of 2-(4-tert-butylphenyl)-1H-benzimidazole (compound 1) or a pharmaceutically acceptable salt thereof; and
70% w/w of a pharmaceutically acceptable carrier matrix.

18. The amorphous solid dispersion of claim 16 or 17, wherein the pharmaceutically acceptable carrier matrix comprises a polyvinyl caprolactam-polyvinyl acetate-polyethylene glycol graft co-polymer, and amorphous silicon dioxide.

19. An amorphous solid dispersion comprising:
10-40% w/w of 2-(4-tert-butylphenyl)-1H-benzimidazole (compound 1) or a pharmaceutically acceptable salt thereof; and
60 to 90% of a pharmaceutically acceptable carrier matrix;
wherein the amorphous solid dispersion is prepared by hot-melt extrusion.

20. The amorphous solid dispersion of claim 19, comprising:
25% w/w of 2-(4-tert-butylphenyl)-1H-benzimidazole (compound 1) or a pharmaceutically acceptable salt thereof; and
75% w/w of a pharmaceutically acceptable carrier matrix.

21. The amorphous solid dispersion of claim 19 or 20, wherein the pharmaceutically acceptable carrier matrix comprises a mixture of:
25% w/w of a copovidone polymer;
25% w/w of a povidone polymer; and
25% w/w of a polyvinyl caprolactam-polyvinyl acetate-polyethylene glycol graft co-polymer.

22. A pharmaceutical composition comprising an amorphous solid dispersion of any one of claims 1 to 21 and one or more pharmaceutically acceptable excipients.

23. The pharmaceutical composition of claim 22, comprising 30 to 50% w/w of the amorphous solid dispersion.

24. The pharmaceutical composition of claim 23, comprising 35 to 45% w/w of the amorphous solid dispersion.

25. The pharmaceutical composition of claim 24, comprising 40% w/w of the amorphous solid dispersion.

26. The pharmaceutical composition of any one of claims 22 to 25, wherein one or more pharmaceutically acceptable excipients is selected from diluents, binders, disintegrants, lubricants, glidants, surfactants, solubilizers, plasticizers, stabilizing agents, antioxidants, sweeteners, and any combination thereof.

27. The pharmaceutical composition of any one of claims 22 to 26, wherein the pharmaceutical composition comprises a diluent.

28. The pharmaceutical composition of claim 27, comprising 40 to 70% w/w of the diluent.

29. The pharmaceutical composition of claim 28, comprising 45 to 65% w/w of the diluent.

30. The pharmaceutical composition of claim 29, comprising 50 to 60% w/w of the diluent.

31. The pharmaceutical composition of any one of claims 22 to 30, wherein the pharmaceutical composition comprises a disintegrant.

32. The pharmaceutical composition of claim 31, comprising 1 to 10% w/w of the disintegrant.

33. The pharmaceutical composition of claim 32, comprising 2 to 8% w/w of the disintegrant.

34. The pharmaceutical composition of claim 33, comprising 4 to 6% w/w of the disintegrant.

35. The pharmaceutical composition of any one of claims 22 to 34, wherein the pharmaceutical composition comprises a lubricant.

36. The pharmaceutical composition of claim 35, comprising 0.5 to 2% w/w of the lubricant.

37. The pharmaceutical composition of claim 36, comprising 0.5 to 1.5% w/w of the lubricant.

38. The pharmaceutical composition of any one of claims 23 to 37, wherein the pharmaceutical composition comprises a glidant.

39. The pharmaceutical composition of claim 38, comprising 0.5 to 2.5% w/w of the glidant.

40. The pharmaceutical composition of claim 39, comprising 1 to 2% w/w of the glidant.

41. The pharmaceutical composition of any one of claims 23 to 40, wherein the pharmaceutical composition comprises a sweetener.

42. The pharmaceutical composition of claim 41, comprising 1 to 5% w/w of the sweetener.

43. The pharmaceutical composition of claim 42, comprising 1 to 2.5% w/w of the sweetener.

44. The pharmaceutical composition of claim any one of claims 22 to 43 comprising:
30 to 50% w/w of the amorphous solid dispersion;
40 to 70% w/w of a diluent;
1 to 10% w/w of a disintegrant;
0.5 to 2% w/w of a lubricant; and
0.5 to 2% w/w of a glidant.

45. The pharmaceutical composition of claim 44 comprising:
35 to 45% w/w of the amorphous solid dispersion;
50 to 60% w/w of a diluent;
4 to 6% w/w of a disintegrant;
0.6 to 1.5% w/w of a lubricant; and
1 to 2% w/w of a glidant.

46. The pharmaceutical composition of claim 44 or 45, further comprising 1 to 5% w/w of a sweetener.

47. The pharmaceutical composition of any one of claims 26 to 30, 44 and 45, wherein the diluent is selected from the group consisting of microcrystalline cellulose, dicalcium phosphate, cellulose, compressible sugars, dibasic calcium phosphate dehydrate, lactose, lactose monohydrate, lactose anhydrous, mannitol, tribasic calcium phosphate, and combinations thereof.

48. The pharmaceutical composition according to any one of claims 31 to 34, 44 and 45, wherein the disintegrant is selected from the group consisting of croscarmellose sodium, crospovidone, modified corn starch, pregelatinized starch, sodium starch glycolate, and combinations thereof.

49. The pharmaceutical composition according to any one of claims 38 to 40, 44 and 45, wherein the glidant is selected from the group consisting of colloidal silicon dioxide, talc, and combinations thereof.

50. The pharmaceutical composition according to any one of claims 35 to 37, 44 and 45, wherein the lubricant is selected from the group consisting of sodium stearyl fumarate, calcium stearate, magnesium stearate, polyethylene glycol, stearic acid, and combinations thereof.

51. The pharmaceutical composition according to any one of claims 41 to 43, wherein the sweetener is an artificial sweetener.

52. A pharmaceutical dosage form comprising an amorphous solid dispersion of any one of claims 1 to 21 or a pharmaceutical composition of any one of claims 22 to 51.

53. The pharmaceutical dosage form of claim 52, wherein the 2-(4-tert-butylphenyl)-1H-benzimidazole (compound 1) is present in an amount of from 50 mg to 500 mg.

54. The pharmaceutical dosage form of claim 53, wherein the 2-(4-tert-butylphenyl)-1H-benzimidazole (compound 1) is present in an amount of from 100 mg to 450 mg.

55. The pharmaceutical dosage form of claim 54, wherein the 2-(4-tert-butylphenyl)-1H-benzimidazole (compound 1) is present in an amount of 180 mg.

56. The pharmaceutical dosage form of claim 54, wherein the 2-(4-tert-butylphenyl)-1H-benzimidazole (compound 1) is present in an amount of 270 mg.

57. The pharmaceutical dosage form of claim 54, wherein the 2-(4-tert-butylphenyl)-1H-benzimidazole (compound 1) is present in an amount of 400 mg.

58. The pharmaceutical dosage form of claim 54, wherein the 2-(4-tert-butylphenyl)-1H-benzimidazole (compound 1) is present in an amount of 450 mg.

59. The pharmaceutical dosage form of any one of claims 52 to 58, wherein the dosage form is granules, a powder, an oral disintegrating tablet (ODT), or an oral suspension.

60. The pharmaceutical dosage form of claim 59, wherein the dosage form is a powder.

61. The pharmaceutical dosage form of claim 59, wherein the dosage form is granules.

62. The pharmaceutical dosage form of claim 61, wherein the granules are obtained by dry granulation.

63. The pharmaceutical dosage form of any one of claims 52 to 62, wherein oral administration of said pharmaceutical dosage form to a selected human subject group produces in said subject group:
an enhanced maximum blood plasma concentration (Cₘₐₓ) for compound 1 that is greater than that achieved with a control formulation of compound 1 (e.g., MC formulation of compound 1);
an area under the curve (AUC) for compound 1 that is at greater than that achieved with a control formulation of compound 1; and
an area under the curve (AUC) for a compound 1 metabolite (e.g., TQS-621) that is greater than that achieved with a control formulation of compound 1.

64. A kit comprising:
a pharmaceutical dosage form of any one of claims 52 to 63; and
instructions for oral administration of the dosage form, wherein the instructions indicate that the dosage form can be reconstituted in a food or beverage.

65. A method of delivering a therapeutically effective amount of compound 1 to a subject in need thereof, comprising orally administering to a subject in need thereof a pharmaceutical dosage form according to any one of claims 52 to 62.

66. The method of claim 65, wherein orally administering to a subject in need thereof the pharmaceutical dosage form achieves:
an enhanced maximum blood plasma concentration (Cₘₐₓ) for compound 1 that is greater than that achieved with a control formulation of compound 1 (e.g., MC formulation of compound 1);
an area under the curve (AUC) for compound 1 that is at greater than that achieved with a control formulation of compound 1; and
an area under the curve (AUC) for a compound 1 metabolite (e.g., TQS-621) that is greater than that achieved with a control formulation of compound 1.
